(19) United States
(12) Patent Application Publication
(10) Pub. No.: US 2009/0062228 A1
(43) Pub. Date: Mar. 5, 2009

(54) piRNA and uses related thereto

(76) Inventors: Gregory J. Hannon, Huntington, NY (US); Michelle A. Carmell, Dorchester, MA (US); Angelique Girard, Cold Spring Harbor, NY (US); Alexei Aravin, Huntington, NY (US); Julius Brennecke, Cold Spring Harbor, NY (US)

Correspondence Address: ROPES & GRAY LLP, PATENT DOCKETING 39/41, ONE INTERNATIONAL PLACE, BOSTON, MA, 02110-2624, US

(21) Appl. No.: 12/075,121

(22) Filed: Mar. 7, 2008

Related U.S. Application Data
(60) Provisional application No. 60/905,773, filed on Mar. 7, 2007.

(51) Int. Cl. A61K 31/7105; C12N 5/06; C07H 21/02; C12Q 1/68; C40B 40/08; C40B 30/04; A61P 43/00

(52) U.S. Cl. 514/44; 435/375; 536/24.5; 536/24.3; 435/6; 506/17; 506/9

(57) ABSTRACT

The invention relates to small single stranded RNAs and analogs thereof (collectively “piRNA” herein), compositions comprising such piRNAs, and their uses in regulating target gene expression or as markers for certain disease states.

### REFERENCE TO RELATED APPLICATIONS

This application claims the benefit of the filing date under 35 U.S.C. § 119(e) of U.S. Provisional Application No. 60/905,773, filed on Mar. 7, 2007, the entire content of which is incorporated herein by reference.

### BACKGROUND OF THE INVENTION

Mobile genetic elements, or their remnants, can be found in the genomes of nearly every living organism. The potential negative effect of mobile elements on the fitness of their hosts necessitates the development of strategies for transposon control. This is particularly important in the germline, where transposon activity can create a substantial mutational burden that would accumulate with each passing generation. However, positive aspects of coexistence with mobile elements have also been posited (reviewed in Brookfield, 2005). For example, mobile elements have been proposed to aid in driving genome evolution and in promoting speculation (Han and Boeke, 2005; Kazazian, 2004). Moreover, repetitive elements have been exploited by their hosts for gene regulation and genome organization, with essential collections of repeat sequences at Drosophila telomeres being one example of the latter (Pardue and DeBaryshe, 2003). Thus, tightly regulated transposon activity may allow the relationship of the mobile element to its host to be of a partially symbiotic nature rather than a purely parasitic one, at least as considered on an evolutionary time scale.

Hybrid dysgenesis is classic paradigm for the deleterious effects of colonization of a host by an uncontrolled mobile element. The progeny of intercrosses between certain Drosophila strains reproducibly show high germline mutation rates with elevated frequencies of chromosomal abnormalities and partial or complete sterility (Kidwell et al., 1977, reviewed in Bucheton, 1990; Castro and Carareto, 2004). Studies of the molecular basis of this phenomenon linked the phenotype to mobilization of transposons (Pelisson, 1981; Rubin et al., 1982). Most instances of hybrid dysgenesis result from the activation of a single transposable element family (Bingham et al., 1982; Bucheton et al., 1984). However, one system of hybrid dysgenesis in D. virilis is characterized by the simultaneous activation of multiple families of unrelated elements (Petrov et al., 1995).

For each combination that produces hybrid dysgenesis, one strain is generally classified as the “inducer”, while the other is termed “reactive” (Bregliano et al., 1980). Depending upon the transposon system, the nomenclature may differ; for example, M-cytotype strains are permissive for P-element transposition while P-cytotype strains are restrictive. The dysgenic phenotype is invariably produced when a reactive female is crossed with an inducer male but is not observed in the reciprocal cross (Pelisson, 1981; Simmons et al., 1980). In general, reactive strains are those that have not recently been exposed to a particular transposon and are therefore devoid of full-length transposon copies. In contrast, inducer strains contain functional transposons to which the strain has developed an active resistance. This active suppression mechanism keeps frequencies of transposition very low in crosses between animals that have both established control over a particular element.

During a dysgenic cross, the transposon carried by the inducer male becomes active in the germline of the progeny of the reactive female. For reasons that are not yet completely understood, transposon activation causes a variety of abnormalities in reproductive tissues, ultimately resulting in sterility (Engels and Preston, 1979). In females, sterility results not only from the direct impact on the parent but also from embryonic developmental defects in the progeny of the affected animal that likely result from alterations in the organization of the oocyte. Since the dysgenic phenotype is often not completely penetrant a fraction of the progeny from affected females survive to adulthood. These animals can develop resistance to the mobilized element, although in many cases, transposon resistance takes several generations to become fully established (Pelisson and Bregliano, 1987). It is important to note that immunity to transposons can only be passed through the female germline, indicating both cytoplasmic and genetic components to inherited resistance (Bregliano et al., 1980).

Studies of hybrid dysgenesis have served a critical role in revealing mechanisms of transposon control in flies. In general, two seemingly contradictory, models have emerged for acquired transposon resistance. The first model correlates resistance with an increasing copy number of the mobile element. A second, alternative model suggests that discrete genomic loci encode transposon resistance.

The first model is supported by studies of the I-element. Crossing a male carrying full-length copies of the I-element to an inexperienced female leads to I mobilization and hybrid dysgenesis (Bregliano et al., 1980; Bucheton et al., 1984). The number of I copies builds during subsequent crosses of surviving female progeny until it reaches an average of 10-15 copies per genome (Pelisson and Bregliano, 1987). At this point, I mobility is suppressed and the initially naïve strain becomes an inducer strain. Thus, in these studies, the gradual increase in I-element copy number over multiple generations was implicated in the development of transposon resistance.

The second model, which attributes transposon resistance to specific loci in the host genome, is illustrated by studies of gypsy transposon control (reviewed in Bucheton, 1995). Specifically, genetic mapping of gypsy resistance determinants led to a discrete locus in the pericentric beta-heterochromatin of the X chromosome that was named flamenco (Pelisson et al., 1994). Females carrying a permissive flamenco allele showed a dysgenic phenotype when crossed to males carrying functional gypsy elements. In contrast, a female carrying a restrictive flamenco allele could suppress gypsy transposition, but only if that allele had been maternally transmitted (Prud'homme et al., 1995). Permissive flamenco alleles are present in natural Drosophila populations but can also be produced by insertional mutagenesis of animals carrying a restrictive flamenco allele (Robert et al., 2001). Despite these studies, and extensive deletion mapping over the flamenco locus, no protein-coding gene in this region has yet been tied to gypsy resistance.

For P-elements, a protein repressor of transposition has been identified as a 66 kD version of the P-element transposase. This protein is encoded by an incompletely spliced version of the P genomic transcript and has been proposed to act as the mediator of P-element resistance (Misra and Rio, 1990; Robertson and Engels, 1989). Increases in P-element copy number were proposed to cause titration of limiting cellular factors essential for proper P-element splicing. When these factors became limiting, production of the unspliced transcript led to the synthesis of a repressor that resulted in a self-imposed limitation on P-element activity. This predicted that P-element resistance would be determined primarily by copy number and would be independent of the precise genomic positions into which P had inserted.

The preceding conclusion was challenged by studies of resistance determinants in inbred lines (Biemont et al., 1990). These revealed that the insertion of P-elements into specific genomic loci provides a potent signal that represses further P-element activity. By following P-cytotype through successive outcrosses, P insertions near the left telomere of X (cytological position 1A) were found to be sufficient for conferring P-element resistance when maternally inherited. Studies of wild isolates carrying the P-cytotype (e.g., Lerak-18 and Epernay-Champagne), also indicated that P-element resistance could be conferred by only one or two copies, of a P element present at 1A (Ronsseray et al., 1991). Additionally, several groups isolated insertions of incomplete P-elements into this same cytological location that also acted as dominant suppressors of transposition (Marin et al., 2000; Stuart et al., 2002). Importantly, in these last cases, the defective P-elements were missing the coding sequences for the repressor fragment of transposase. Thus, these studies were collectively consistent with resistance being tied to the insertion of a P-element into a specific site rather than to P-element copy number or an encoded protein product.

Both models of acquired transposon resistance, those determined by specific genomic loci and those caused by copy-number dependent responses, can be rationalized as working through small RNA-based regulatory pathways. Evidence in support of this hypothesis comes from three separate observations. First, copy-number dependent silencing of mobile elements is reminiscent of observations of copy-number dependent transgene silencing in plants (transgene co-suppression) (Smyth, 1997) and Drosophila (Pal-Bhadra et al., 1997). In both of those cases, silencing occurs through an RNAi-like response where high-copy transgenes provoke the generation of small RNAs, presumably through a double-stranded RNA intermediate (Hamilton and Baulcombe, 1999; Pal-Bhadra et al., 2002). Second, mutations affecting proteins that have been linked to the RNAi-like responses impact transposon mobility in Drosophila (Kalmykova et al., 2005; Sarot et al., 2004; Savitsky et al., 2006) and Celegans (Ketting et al., 1999; Tabara et al., 1999). Finally, small RNAs corresponding to transposons and repeats have been detected in Drosophila (Aravin et al., 2003; Aravin et al., 2001). Aravin and colleagues first noted that Drosophila small RNAs matching transposon sequences were prevalent in early embryos and testes but were less common in late stage larvae and adults (Aravin et al., 2003). These RNAs (termed repeat-associated siRNAs or rasiRNAs) were slightly larger than microRNAs, being 24-26 nucleotides in length. Subsequently, rasiRNAs were also found in Zebrafish (Chen et al., 2005), suggesting that the RNAi pathway may play a conserved role in transposon control in animals analogous to its well established role in regulating mobile elements in plants.

At the core of the RNAi machinery are the Argonaute proteins, which directly bind to small RNAs and use these as guides to the identification of silencing targets (Liu et al., 2004). Argonaute proteins can enforce silencing directly by cleaving bound RNA targets via an endogenous RNAse H-like domain (Liu et al., 2004; Rivas et al., 2005). In animals, the Argonaute superfamily can be divided into two clades (Carmell et al., 2002). One contains the Argonautes themselves, which act with microRNAs and siRNAs to mediate gene silencing. The second contains the Piwi proteins, which incorporate all Argonaute signature domains but which, until recently, were left without identified small RNA partners. Genetic studies have implicated Piwi clade proteins in germline integrity (Cox et al., 1998; Harris and Macdonald, 2001). For example, mutation of the Piwi gene itself causes female sterility and loss of germline stem cells (Cox et al., 1998; Lin and Spradling, 1997). Another Piwi family member, Aubergine, is a spindle-class gene that is required in the germline for the production of functional oocytes (Harris and Macdonald, 2001). A third Drosophila Piwi gene, Ago3, has yet to be studied. Mutation of Piwi family genes can also affect the transposition of mobile elements. For example, mutations in Piwi mobilize gypsy (Sarot et al., 2004), and Aubergine mutations impact repression of TART (Savitsky et al., 2006) and P-element transposition (Reiss et al., 2004).

A direct link between small RNAs and Drosophila Piwi proteins was made recently through the observation that both Piwi and Aubergine complexes contain rasiRNAs (Saito et al., 2006; Vagin et al., 2006). Using tiling oligonucleotide microarrays corresponding to consensus transposon sequences, Piwi and Aubergine were found to bind rasiRNAs targeting a number of mobile and repetitive elements, including roo, I, gypsy and the testis-specific Su(Ste) locus (Vagin et al., 2006). Interestingly, these complexes were enriched for RNAs from the antisense strand of the transposon, as might be expected if the complexes were actively involved in silencing transposons by recognition of their RNA products. Small scale sequencing of RNAs associated with Piwi also indicated binding to rasiRNAs derived from a wide variety of transposons and repeats, with a preference for antisense small RNAs in the former case (Saito et al., 2006). Neither study indicated that Piwi bound detectably to microRNAs.

Recently, another class of small RNAs, the Piwi-interacting RNAs (piRNAs), was identified through association with Piwi proteins in mammalian testes (Aravin et al., 2006; Girard et al., 2006; Grivna et al., 2006; Lau et al., 2006). These RNAs range from 26-30 nucleotides in length and are produced from discrete loci. Generally, genomic regions spanning 50-100 kB in length give rise to abundant piRNAs with profound strand asymmetry. Although the piRNAs themselves are not conserved, even between closely related species, the positions of piRNA loci in related genomes are conserved, with virtually all major piRNA-producing loci having synthetic counterparts in mice, rats and humans (Girard et al., 2006). Interestingly, the loci and consequently the piRNAs themselves are relatively depleted of repeat and transposon sequences, with only 17% of human piRNAs corresponding to known repetitive elements as compared to a nearly 50% repeat content for the genome as a whole. Despite the apparent differences in the content of RNA populations associated with Piwi proteins in mammals and Drosophila, Piwi family proteins share essential roles in gametogenesis, with all three murine family members, Miwi2, Mili, and Miwi, being required for male fertility.

### SUMMARY OF THE INVENTION

The invention in general relates to the use of single-stranded RNA constructs (natural or modified), known herein as “piRNA,” to modulate target gene expression.

Thus in one aspect, the invention provides a method for regulating the expression of a target gene in a cell, comprising introducing into the cell a small single stranded RNA or analog thereof (piRNA) that: (i) selectively binds to proteins of the Piwi or Aubergine subclasses of Argonaute proteins relative to the Ago3 subclass of Argonaute proteins, (ii) forms an RNP complex (piRC) with the Piwi or Aubergine proteins, and, (iii) induces transcriptional and/or post-transcriptional gene silencing, wherein the piRNA induces transcriptional and/or post-transcriptional gene silencing of the target gene.

In certain embodiments, the k_{d} for binding of the piRNA to Piwi and/or Aubergine subfamily of proteins is at least about 50%, 100%, 2-fold, 5-fold, 10-fold, 20-fold, 50-fold, 100-fold, 1000-fold or lower (tighter or more selective binding) than that for binding to the Ago3 subfamily of proteins.

In certain embodiments, the piRNA is about 25-50 nucleotides in length, about 25-39 nucleotides in length, or about 26-31 nucleotides in length.

In certain embodiments, the minimal length of the piRNA is about 24, 25, 26, 27, 28, 29, 30, 31, 32, 33, 34, or 35 nucleotides in length.

In certain embodiments, the maximum length of the piRNA is no more than 100, 90, 80, 70, 60, 50, 45, 44, 43, 42, 41, 40, 39, 38, 37, 36, 35, 34, 33, 32, 31, 30, 29, 28, 27, 26, 25 nucleotides in length.

In certain embodiments, the piRNA is processed from a long precursor RNA, which may be transcribed in vitro or in vivo from coding sequence on a vector (a plasmid, an expression vector, a retroviral vector, a lentiviral vector, etc.).

In certain embodiments, the piRNA preferentially associates with the MILI protein and is about 26-28 nucleotides in length.

In certain embodiments, the piRNA comprises a nucleotide sequence that hybridizes under physiologic conditions of a cell to the nucleotide sequence of at least a portion of a genomic sequence of the cell to cause down-regulation of transcription at the genomic level, or to cause down-regulation of transcription of an mRNA transcript for a target gene.

In certain embodiments, the piRNA comprises no more than 1 in 5 basepairs of nucleotide mismatches with respect to the target gene mRNA transcript.

In certain embodiments, the piRNA is greater than 90% identical to the portion of the target gene mRNA transcript to which it hybridizes.

In certain embodiments, the piRNA comprises one or more modifications on phosphate-sugar backbone or on nucleosides.

In certain embodiments, the modifications on phosphate-sugar backbone comprise phosphorothioate, phosphoramidate, phosphodithioates, or chimeric methylphosphonate-phosphodiester linkages.

In certain embodiments, the modifications on nucleosides comprise 2′-methoxyethoxy, 2′-methyl-thio-ethyl, 2′-deoxy-2′-fluoro, 2′-deoxy-2′-chloro, 2-azido, 2′-O-trifluoromethyl, 2′-O-ethyl-trifluoromethoxy, 2′-O-difluoromethoxy-ethoxy, 4′-thio, or 2′-O-methyl modifications.

In certain embodiments, the piRNA comprises a terminal cap moiety at the 5′-end, the 3′-end, or both the 5′ and 3′ ends.

In certain embodiments, the piRNA comprises a 5′-uracil (5′-U) residue.

In certain embodiments, the target gene is an insect-specific gene.

In certain embodiments, the cell is a stem cell, such as an embryonic or adult stem cell.

In certain embodiments, the cell is in culture or in a whole organism (in vivo).

In certain embodiments, the target gene is required or essential for cell growth and/or development, for mRNA degradation, for translational repression, or for transcriptional gene silencing (TGS).

Another aspect of the invention provides a composition or therapeutic formulation comprising the subject piRNA, pharmaceutically acceptable salts, esters or salts of such esters, or bioequivalent compounds thereof, admixed, encapsulated, conjugated or otherwise associated with liposomes, polymers, receptor targeted molecules, oral, rectal, topical or other formulations that assist uptake, distribution and/or absorption.

In certain embodiments, the composition or therapeutic formulation further comprises penetration enhancers, carrier compounds, and/or transfection agents.

Another aspect of the invention provides a polynucleotide comprising two or more concatenated piRNAs, each of said piRNAs comprise a small single stranded RNA or analog thereof that: (i) selectively binds to proteins of the Piwi or Aubergine subclasses of Argonaute proteins relative to the Ago3 subclass of Argonaute proteins, (ii) forms an RNP complex (piRC) with the Piwi or Aubergine proteins, and, (iii) induces transcriptional and/or post-transcriptional gene silencing.

In certain embodiments, the piRNAs are of the same or different sequences.

Another aspect of the invention provides a polynucleotide encoding one or more subject piRNA(s) or precursor(s) thereof, wherein said piRNA(s) are transcribed from said polynucleotide, or wherein said precursor(s), when transcribed from said polynucleotide, are metabolized by a cell comprising the polynucleotide to give rise to the subject piRNA(s).

Another aspect of the invention provides a probe comprising a polynucleotide that hybridizes to the subject piRNA.

In certain embodiments, the polynucleotide is an RNA.

In certain embodiments, the probe comprises at least about 8-22 contiguous nucleotides complementary to the subject piRNA.

Another aspect of the invention provides a plurality of the subject probes, for detecting two or more piRNA sequences in a sample.

Another aspect of the invention provides a composition comprising the subject probe, or the plurality of probes.

Another aspect of the invention provides a method of detecting the presence or absence of one or more particular piRNA sequences in a sample from the genome of a patient or subject, comprising contacting the sample with the subject probe, or the plurality of probes.

In certain embodiments, the sample is a cell or a gamete of the patient or subject.

Another aspect of the invention provides a biochip comprising a solid substrate, said substrate comprising a plurality of probes for detecting the subject piRNA.

In certain embodiments, each of the probes is attached to the substrate at a spatially defined address.

In certain embodiments, the biochip comprises probes that are complementary to a variety of different piRNA sequences.

In certain embodiments, the variety of different piRNA sequences are differentially expressed in normal versus disease tissue, or at different stages of development.

Another aspect of the invention provides a method of detecting differential expression of disease-associated piRNA(s), comprising: (1) contacting a disease sample with a plurality of probes for detecting piRNA sequences, (2) contacting a control sample with the plurality of probes, and, (3) identifying one or more of piRNA sequences that are differentially expressed in the disease sample as compared to the control sample, thereby detecting differential expression of disease-associated piRNA(s).

Another aspect of the invention provides a method of identifying a compound that modulates a pathological condition or a cell/tissue development pathway, the method comprising: (1) providing a cell that expresses one or more piRNAs as markers for a particular cell phenotype or cell fate of the pathological condition or the cell/tissue development pathway; (2) contacting the cell with a candidate agent; and, (3) measuring the expression level of at least one said piRNAs, wherein a change in the expression level of at least one said piRNAs indicates that the candidate agent is a modulator of the pathological condition or the cell/tissue development pathway.

It is contemplated that all embodiments of the invention, including those described under different aspects of the invention, can be combined with other embodiments of the invention whenever applicable.

### BRIEF DESCRIPTION OF THE DRAWINGS

FIG. 1 shows the size distribution of sequenced piRNAs specifically bound by the three Piwi family members. The left-most curve is for Ago3-IP, the middle curve is for Aub-IP, and the right-most curve is for Piwi-IP.

FIG. 2 shows a slicer-mediated amplification loop for piRNAs, with an individual example of two cloned piRNAs which overlap with the characteristic 10 nt offset (with the 5′U of the Aub bound roo antisense piRNA, and the A at position 10 of the Ago3 bound roo sense piRNA).

FIG. 3 is a ClustalW alignment of the three Drosophila Piwi family proteins. The Ago3 sequence represents the largest open reading frame in the putative full length cDNA clone RE57814. The N-terminal 16, 16, and 14 peptides are used for polyclonal antibody production of Piwi, Aub, and Ago 3, respectively. PAZ and PIWI domains are shown in the first and second boxes, respectively. The position of the catalytic DDH residues essential for slicer mediated cleavage are indicated by arrowheads. Note, that although Piwi contains a DDK motif, Slicer activity has been demonstrated for this protein (Saito et al., 2006).

FIG. 4 is a schematic drawing showing properties and biogenesis of piRNAs. FIG. 4A shows features of Aub- and AGO3-associated piRNAs in Drosophila. Indicated are the 5′ U bias in Aub-bound piRNAs, the 10A bias in AGO3-bound piRNAs, the 5′ phosphate, and the 3′ O-methylation. FIG. 4B shows the Ping-Pong model of piRNA biogenesis in Drosophila. Primary piRNAs are generated by an unknown mechanism and/or are maternally deposited. Those with a target are specifically amplified via a Slicer-dependent loop involving AGO3 and Aub.

FIG. 5 shows a Piwi-mediated piRNA amplification loop in mammals. L1 (FIG. 5A) and IAP (FIG. 5B) piRNAs were aligned to their consensus sequences allowing up to three mismatches, and distances separating 5′ ends of complementary piRNA were plotted. nt, nucleotide. Nucleotide biases were calculated for L1 (FIG. 5C) and IAP (FIG. 5D) piRNAs analyzed in FIG. 5A and FIG. 5B. The fraction of A at position 10 was plotted both for piRNA classes that contain and lack a 5′ U. For each bar, the percentage of U or A residues that would be expected by random sampling is indicated by a solid line across the bar.

### DETAILED DESCRIPTION OF THE INVENTION

### 1. Overview

The invention in general relates to the Piwi clade of Argonaute superfamily proteins that are somewhat related to the Argonaute clade proteins, the latter of which are involved in RNA-interference (RNAi) using siRNA and microRNA. Historically, RNAi has been defined as a response to double-stranded RNA. However, some small RNA species (such as the subject piRNA) may not arise from double-stranded RNA precursors. Yet, like microRNAs (miRNAs) and small interfering RNAs (siRNAs), such piRNA species guide certain Piwi clade Argonaute superfamily proteins to silence target genes through complementary base-pairing. Silencing can be achieved by co-recruitment of accessory factors or through the activity of Argonaute superfamily proteins, which often have endonucleolytic activity.

Thus one aspect of the invention relates to the use of small single stranded RNAs and analogs thereof (collectively “piRNA” herein) that (i) selectively bind to proteins of the Piwi and Aubergine subclasses of Argonaute superfamily proteins, e.g., relative to binding to the Ago3 subclass proteins, (ii) form an RNP complex (piRC) with the Piwi/Aubergine proteins, and (iii) induce transcriptional and/or post-transcriptional gene silencing. Such piRNA may be used to silence target gene expression in a host cell (such as cultured cell) or animal, including insets to mammalian hosts.

In certain embodiments, the piRNA is 25-50 nucleotides in length, and more preferably 25-39 nucleotides in length, and even more preferable 26-31 nucleotides in length. In one embodiment, the piRNA associates with a Piwi protein and is 29-31 nucleotides in length. In other embodiments, the piRNA preferentially associates with the MILI protein and is slightly shorter, e.g., 26-28 nucleotides in length.

In still other embodiments, multiple piRNA (of the same or different sequence) can be provided as single concatenated nucleic acid species.

In yet other embodiments, the piRNA or multiple piRNA species can be provided as an “encoded” piRNA, i.e., as “coding” sequence on an expression construct that, when transcribed, produces the piRNA species as a transcript or a transcript that is a precursor which is metabolized by the cell to give rise to a piRNA species.

In certain embodiments, the piRNA contains a nucleotide sequence that hybridizes under physiologic conditions of the cell to the nucleotide sequence of at least a portion of a genomic sequence to cause down-regulation of transcription at the genomic level, or an mRNA transcript for a gene to be inhibited (i.e., the “target” gene). The piRNA need only be sufficiently similar to natural RNA that it has the ability to mediate PIWI-dependent gene silencing. Thus, the invention has the advantage of being able to tolerate sequence variations that might be expected due to genetic mutation, strain polymorphism or evolutionary divergence. The number of tolerated nucleotide mismatches between the target sequence and the piRNA sequence is preferably no more than 1 in 5 basepairs. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the piRNA and the portion of the target gene is preferred. Alternatively, the piRNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of piRNAs can be carried out by chemical synthetic methods or by recombinant nucleic acid techniques. Endogenous RNA polymerase of the treated cell may mediate transcription in vivo, or cloned RNA polymerase can be used for transcription in vitro. The piRNAs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The piRNA may be produced enzymatically or by partial/total organic synthesis, any modified ribonucleotide can be introduced by in vitro enzymatic or organic synthesis.

Methods of chemically modifying RNA molecules can be adapted for modifying piRNAs (see, for example, Heidenreich et al. (1997) Nucleic Acids Res, 25: 776-780; Wilson et al. (1994) J. Mol Recog 7: 89-98; Chen et al. (1995) Nucleic Acids Res 23: 2661-2668; Hirschbein et al. (1997) Antisense Nucleic Acid Drug Dev 7: 55-61). Merely to illustrate, the backbone of a piRNA can be include one or more modified internucleotidic linkage, such as phosphorothioate, phosphoramidate, phosphodithioates, chimeric methylphosphonate-phosphodiesters linkages. The piRNA can also be derived using locked nucleic acid (LNA) nucleotides, as well as using modified ribose bases such as 2′-methoxyethoxy nucleotides; 2′-methyl-thio-ethyl nucleotides, 2′-deoxy-2′-fluoro nucleotides, 2′-deoxy-2′-chloro nucleotides, 2-azido nucleotides, 2′-O-trifluoromethyl nucleotides, 2′-O-ethyl-trifluoromethoxy nucleotides, 2′-O-difluoromethoxy-ethoxy nucleotides, 4′-thio nucleotides and 2′-O-methyl nucleotides. The piRNA can include a terminal cap moiety at the 5′-end, the 3′-end, or both of the 5′ and 3′ ends.

In certain embodiments, the piRNA includes a 5′-U residue.

The subject piRNAs regulate processes essential for cell growth and development, including messenger RNA degradation, translational repression, and transcriptional gene silencing (TGS). Accordingly, the piRNA molecules of the instant invention provide useful reagents and methods for a variety of therapeutic, prophylactic, veterinary, diagnostic, target validation, genomic discovery, genetic engineering, and pharmacogenomic applications.

In certain embodiments, the subject piRNA can be used for birth control, i.e., to reduce fertility in a patient.

In certain embodiments, the subject piRNA can be used to regulate the growth and/or differentiation state of embryos, in vivo or in culture.

In certain embodiments, the subject piRNA can be used to regulate the growth and/or differentiation state of embryonic or other stem cells, in vivo or in culture.

In certain embodiments, the subject piRNA can be used as an insecticide by utilizing piRNA that are selectively expressed in insects (specific species or generally) relative to mammals.

The piRNAs of the invention may also be admixed, encapsulated, conjugated or otherwise associated with other molecules, molecule structures or mixtures of compounds, as for example, liposomes, polymers, receptor targeted molecules, oral, rectal, topical or other formulations, for assisting in uptake, distribution and/or absorption. The subject piRNAs can be provided in formulations also including penetration enhancers, carrier compounds and/or transfection agents.

Representative United States patents that teach the preparation of such uptake, distribution and/or absorption assisting formulations which can be adapted for delivery of RNA molecules particularly piRNA, include, but are not limited to, U.S. Pat. Nos. 5,108,921; 5,354,844; 5,416,016; 5,459,127; 5,521,291; 51543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The piRNAs of the invention also encompass any pharmaceutically acceptable salts, esters or salts of such esters, or any other compound which, upon administration to an animal including a human, is capable of providing (directly or indirectly) the biologically active metabolite or residue thereof. Accordingly, for example, the disclosure is also drawn to piRNAs and pharmaceutically acceptable salts of the piRNAs, pharmaceutically acceptable salts of such piRNAs, and other bioequivalents.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,NI-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The present invention also provides probes comprising a nucleic acid that hybridizes to a piRNA sequence—i.e., genomic in some embodiments, RNA in other instances. The probe may comprise at least 8-22 contiguous nucleotides complementary to a piRNA sequence. The present invention is also related to a plurality of the probes for detecting two or more piRNA sequences in a sample. The present invention is also related to a composition comprising a probe or plurality of probes. In certain embodiments, the subject probes can be used to assess the presence or absence of particular piRNA sequences in the genome of a patient or subject. In other embodiments, the subject probes can be used to assess the presence or absence of particular piRNA (RNA species) in the cells or gametes of a patient or subject.

The present invention is also related to a biochip comprising a solid substrate, said substrate comprising a plurality of the piRNA-detecting probes. Each of the probes may be attached to the substrate at a spatially defined address. The biochip may comprise probes that are complementary to a variety of different piRNA sequences, such as may be differentially expressed in normal versus disease tissue or at different stages of development. The present invention is also related to a method of detecting differential expression of a disease-associated piRNA.

The present invention is also related to a method of identifying a compound that modulates a pathological condition or a cell/tissue development pathway. A cell may be provided that is capable of expressing a nucleic acid one or more piRNA as markers for a particular cell phenotype or cell fate. The cell may be contacted with a candidate agent and then measuring the level of expression of each piRNA is measured. A difference in the level of one or more piRNA can be used identify the compound as a modulator of a pathological condition or development pathway associated with the piRNA sequence.

### 2. The Piwi Clade of Proteins

Argonaute proteins, in complex with distinct classes of small RNAs, form the core of the RNA-induced silencing complex (RISC), the RNA-interference (RNAi) effector complex. The Argonaute superfamily segregates into two clades, the Ago lade and the Piwi clade. The single fission yeast Argonaute and all plant family members belong to the Ago clade, whereas ciliates and slime molds contain members of the Piwi clade. Together, these findings indicate that Piwis and Agos are similarly ancient. Animal genomes typically contain members of both clades, and it is becoming clear that this division of Argonautes reflects their underlying biology.

Ago clade proteins complex with microRNAs (miRNAs) and small interfering RNAs (siRNAs), which derive from double-stranded RNA (dsRNA) precursors. miRNA-Ago complexes reduce the translation and stability of protein-coding mRNAs, which results in a regulatory network that impacts ˜30% of all genes.

The Piwi clade is found in all animals examined so far, and all such Piwi clade proteins are within the scope of the invention.

The genomes of multicellular animals encode multiple Piwi proteins. The three Drosophila proteins Piwi, Aubergine, and AGO3 are expressed in the male and female germ lines. These three Drosophila proteins, based on sequence identity and/or functional similarity, define the three subclasses of the Piwi clade proteins.

In general, one function of the Piwi clade proteins are correlated with the emergence of specialized germ cells. For example, expression of the three mouse proteins MIWI (PIWIL1), MILI (PIWIL2), and MIWI2 (PIWIL4) is mainly restricted to the male germ line. Consistent with their expression pattern, Piwi mutant animals exhibit defects in germ cell development. Although some somatic expression of Piwis has been reported, mutant animals lack obvious defects in the soma.

Another function of the Piwi pathway proteins is silencing selfish genetic elements, through interacting with their small RNA partners—Piwi-Interacting RNAs (piRNAs).

In Drosophila, there is a distinct population of Piwi-associated small RNAs that silences target gene expression. For example, the presence of 25- to 27-nucleotide (nt) RNAs homologous to the repetitive Stellate locus was correlated with its silencing, and required the Piwi clade protein Aubergine. Profiling of small RNAs through Drosophila development placed Stellate-specific small RNAs into a broader class, derived from various repetitive elements, called repeat-associated small interfering RNAs (rasiRNAs). A direct interaction between rasiRNAs and Piwi proteins was demonstrated by immunoprecipitation of Piwi complexes.

Small RNAs resembling Drosophila rasiRNAs have also been identified in testes and ovaries of zebrafish, which demonstrates evolutionary conservation of this small RNA class.

Small RNA partners of Piwi proteins were also identified in mammalian testes and termed Piwi-interacting RNAs (piRNAs). Although these RNAs share some features with rasiRNAs, there are also substantial differences, including a dearth of sequences matching repetitive elements. Nonetheless, on the basis of their common features, as used herein, “piRNA” includes all small RNAs in the Piwi clade complexes, with Drosophila rasiRNAs and mammalian piRNAs as specialized subclasses of the subject piRNA.

Piwis and piRNAs form a system distinct from the canonical RNAi and miRNA pathways. No association between Piwis and miRNAs was detected in either fly or mouse, although piRNAs, like miRNAs, carry a 5′ monophosphate group and exhibit a preference for a 5′ uridine residue. In contrast to miRNAs, many of which are conserved through millions of years of evolution, individual piRNAs are poorly conserved even between closely related species. piRNAs in Drosophila and mammals, as well as siRNA-like scan RNAs that bind Piwi proteins in ciliates, are substantially longer (24 to 30 nt) than miRNAs and siRNAs (21 to 23 nt). Unlike animal miRNAs, but similar to plant miRNAs, piRNAs carry a 2′O-methyl modification at their 3′ ends, which is added by a Hen-1 family RNA methyltransferase. Finally, genetic analyses in flies and zebrafish argue against a role for Dicer, a key enzyme in miRNA and siRNA biogenesis, in piRNA production.

The genomic origin of piRNAs is also unique. Most Drosophila piRNAs match repetitive elements and therefore map to the genome in dozens to thousands of locations. Yet mapping of those piRNAs that could be placed uniquely in the genome (e.g., piRNAs from divergent repeat copies) identified a limited set of discrete loci that could give rise to most piRNAs. These were dubbed “piRNA clusters.” piRNA clusters range from several to hundreds of kilobases in length. They are devoid of protein coding genes and instead are highly enriched in transposons and other repeats. The vast majority of transposon content in piRNA clusters occurs in the form of nested, truncated, or damaged copies that are likely not capable of autonomous expression or mobilization. The presence of transposable elements per se is not sufficient for piRNA production. Virtually all piRNA clusters in Drosophila are located in pericentromeric or telomeric heterochromatin, which suggests that chromatin structure may play a role in defining piRNA clusters.

Prominent piRNA loci are also found in mammals and zebrafish. Mammalian piRNAs can be divided into two populations. Pachytene piRNAs appear around the pachytene stage of meiosis, become exceptionally abundant, and persist until the haploid round spermatid stage, after which they gradually disappear during sperm differentiation. Pachytene piRNAs are relatively depleted of repeats, and even those that do match annotated transposons are diverged from consensus, potentially active copies. Prepachytene piRNAs are found in germ cells before meiosis. These share the molecular characteristics of pachytene piRNAs but originate from a different set of clusters that more closely match those of Drosophila and zebrafish in repeat content.

Generally, clusters in flies and vertebrates give rise to piRNAs that associate with multiple Piwi clade proteins. Mouse pachytene piRNAs join both MILI and MIWI complexes. Similarly, Drosophila clusters produce piRNAs, which associate with all three Piwi proteins. However, some clusters generate piRNAs that join specific Piwi proteins, likely because these clusters and the Piwi proteins with which their products associate display specific temporal and special expression patterns. For example, Drosophila piRNAs originating from the flamenco cluster are found almost exclusively in Piwi complexes, and that is the only family member that is present in the somatic cells of the ovary, where flamenco is predominantly expressed.

Unlike trans-acting siRNAs in plants, piRNAs do not arise from clusters in a strictly phased manner but rather originate from irregular positions forming pronounced peaks and gaps of piRNA density. piRNA populations are extremely complex, with recent estimates placing the number of distinct mammalian pachytene piRNAs at >500,000.

Biogenesis of piRNAs does not appear to depend on Dicer. The profound strand asymmetry of mammalian pachytene clusters indicate that piRNAs are not generated from dsRNA precursors. In Drosophila, most piRNA clusters generate small RNAs from both strands; however, there are exceptions, such as the flamenco locus, where piRNAs map almost exclusively to one genomic strand. In zebrafish, piRNAs can map to both genomic strands; however, within any given region of a cluster, only one strand gives rise to piRNAs.

Without wishing to be bound by any particular theory, one model of natural piRNA biogenesis provides the generation of piRNAs by sampling of long single-stranded precursors. According to a second model, piRNAs could be made as primary transcription products. Evidence for the former is the lack of a 5′ triphosphate group and the observation that a single P-element insertion at the 5′ end of the flamenco cluster prevents the production of piRNAs up to 160 kb away. This strongly supports a model in which a single transcript traverses an entire piRNA cluster and is subsequently processed into mature piRNAs.

Processing of small RNAs from long single stranded transcripts is not unprecedented. Indeed, miRNAs are processed from precursors that often span several kilobases and that can encode several individual miRNAs. Pronounced peaks in piRNA density within a cluster also hint at the existence of specific processing determinants. The machinery that produces piRNAs from cluster-derived transcripts is somewhat flexible, as different Piwi proteins in flies and mammals each incorporate a distinct size class of small RNA. Data from flies and mammals suggest a model in which piRNA production begins with single cleavage of a primary piRNA cluster transcript to generate a piRNA 5′ end. piRNAs may be sampled virtually from any position within a cluster with the only preference being a 5′ uridine residue. After incorporation of the cleaved RNA into a Piwi, a second activity generates the 3′ end of the piRNA with the specific size determined by the footprint of the particular family member on the RNA.

Piwi and Aubergine complexes contain piRNAs antisense to a wide variety of Drosophila transposons, and these show the strong 5′-U preference noted for mammalian piRNAs. In contrast, AGO3 associates with piRNAs strongly biased toward the sense strand of transposons and with no 5′ nucleotide preference. piRNAs in AGO3 show a characteristic relation with piRNAs found in Aub complexes, with these small RNAs overlapping by precisely 10 nt at their 5′ ends. Accordingly, the AGO3-bound piRNAs were strongly enriched for adenine at position 10, which is complementary to the 5′ U of Aub-bound piRNAs. These observations indicated the existence of two distinct piRNA populations, possibly with different biogenesis mechanisms, and led to the hypothesis that cluster-derived transcripts and transcripts from active transposons interact through the action of Piwi proteins to form a cycle that amplifies piRNAs that target active mobile elements.

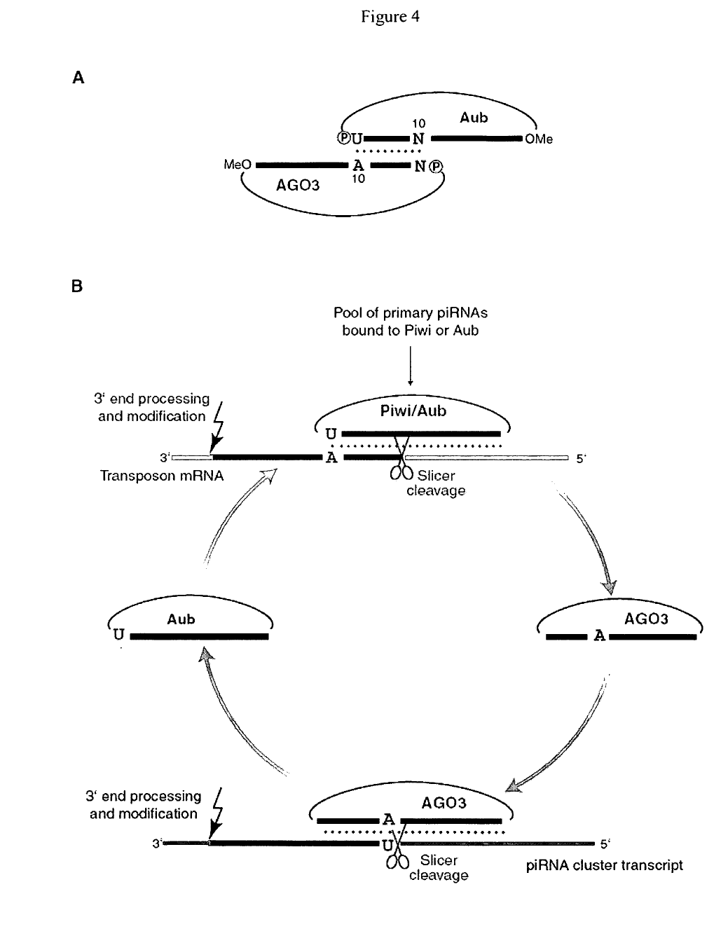

The cycle (called the Ping-Pong amplification loop) (FIG. 4B) begins with a transposon-rich piRNA cluster giving rise to a variety of piRNAs. In most clusters, a random arrangement of transposon fragments would initially produce a mixture of sense and antisense piRNAs, likely populating Piwi and Aub. When encountering a complementary target, a transposon mRNA, Piwi/Aub complexes cleave 10 nt from the 5′ end of their associated piRNA. This not only inactivates the target but also creates the 5′-end of new AGO3-associated piRNA. Loaded AGO3 complexes are also capable of cleaving complementary targets; one place from which such targets could be derived is the clusters themselves.

Cleavage of cluster transcripts by AGO3 would then generate additional copies of the original antisense piRNA, which would enter Aub and become available to silence active transposons. The combination of these steps can form a self-amplifying loop. Signatures of this amplification loop are also apparent in zebrafish and in mammalian prepachytene piRNAs.

Studies of piRNAs have pointed to a conserved function of Piwi clade proteins and their associated piRNAs in the control of mobile genetic elements, and this is consistent with the defects in transposon suppression observed in Piwi mutants. For example, The flamenco locus maps to the pericentromeric heterochromatin on the X chromosome of Drosophila, and represses transposition of the retrotransposons gypsy, ZAM, and Idefix. Genetic analysis failed to reveal a protein-coding gene underlying flamenco function; however, the discovery that flamenco is a major piRNA cluster provided a molecular basis for its ability to suppress several unrelated retroelements. flamenco spans at least 180 kb and is highly enriched in many types of repetitive elements, including multiple fragments of gypsy, ZAM, and Idefix. In flamenco mutants, gypsy is desilenced, and essentially all piRNAs derived from this cluster are lost. Thus, flamenco is an archetypal piRNA cluster that encodes a specific silencing program, which is parsed by processing into individual, active small RNAs that exert their effects on loci located elsewhere in the genome.

Genetic studies of Piwi mutants also suggested involvement in germline development in both invertebrates and vertebrates. Drosophila piwi is required in germ cells, as well as in somatic niche cells, for regulation of cell division and maintenance of germline stem cells. The aubergine phenotype resembles so-called spindle-class mutants that demonstrate meiotic progression defects. The defects in spindle-class mutants are a direct consequence of Chk2 and ATR (ataxia telangiectasia mutated and Rad3-related) kinase dependent meiotic checkpoint activation, and the phenotypes of aub mutants are partially suppressed in animals defective for this surveillance pathway.

In mice, loss of individual Piwi proteins causes spermatogenic arrest. In Miwi mutants, germ cells are eliminated by apoptosis after the haploid, round spermatid stage. However, in Mili and Miwi2 mutants, earlier defects appear as meiosis is arrested around the pachytene stage. In flies, mammals, and zebrafish, no phenotypic abnormalities have yet been detected outside of the germ line, in accord with the expression pattern of Piwis.

Overall, genetic and biochemical data indicate that a substantial component of Piwi biology is dedicated to transposon control. The diverse effects of Piwi mutations can be largely explained through the actions of Piwi proteins in transposon control. In Drosophila, studies of hybrid dysgenesis linked transposon activation to severely impaired gametogenesis. Mutation of a single piRNA cluster, flamenco, results in defects in germ and follicle cell development and complete sterility. Defects in aub mutants are linked to DNA damage checkpoint signaling that is probably activated in response to doublestrand breaks arising from transposon activity. In mammals, germ cell loss in Mili and Miwi2 mutants has been correlated with transposon activation. Other studies also support the idea that severe defects in germ cell development can be a direct consequence of transposon activation. For example, Dnmt3L deficient animals show demethylation of transposable elements, which lead to their increased expression, as well as meiotic catastrophe and germ cell loss, a combination of phenotypes similar to those seen in Mili and Miwi2 mutants.

One possible exception to this paradigm may be the mammalian pachytene piRNAs. The extreme diversity of pachytene piRNAs may allow MIWI and MILI complexes to exert broad effects on the transcriptome through a miRNA-like mechanism.

It is becoming increasingly clear that an ancient and conserved function of the Piwi and piRNA pathway is to protect the genome from the activity of parasitic nucleic acids. Even in ciliates, which diverged earlier than the common ancestor of plants and animals, parallels to the piRNA pathways of flies and mammals are clear. In Tetrahymena, the scanning hypothesis for DNA elimination suggests that a complex population of small RNAs is first generated from the micronuclear genome and subsequently filtered through interactions with the old macronuclear genome. The small RNAs that emerge from this process specify repeat silencing, in this case by elimination from the newly forming and transcriptionally active macronucleus. DNA elimination depends upon a Piwi protein, Twi1, but unlike the case in vertebrates and Drosophila, also on a Dicer protein.

Comparisons to ciliates reveal that, during evolution, the core Piwi and piRNA machinery may have adopted both different strategies for producing and filtering small RNA triggers and different strategies for ultimately silencing targets. In Drosophila, the Ping-Pong model strongly suggests a post-transcriptional component to transposon silencing. However there is also evidence for impacts of Piwi proteins on chromatin states. In mammals, Piwi proteins have been implicated in DNA methylation, a function that may be exerted either directly or indirectly. Plants lack Piwi proteins and have adapted a different RNAi-based strategy for transposon control. In Arabidopsis, the Ago subfamily protein Ago4 is programmed with a complex set of transposon-derived small RNAs. In contrast to flies and mammals, in which piRNA loci serve as a genetically encoded reservoir of resistance to mobile elements, each individual transposon copy seems to produce small RNAs in plants. There are hints that chromatin marks may help to concentrate small RNA production at particular sites. This resembles the situation for centromeric repeats in S. pombe where specific histone modifications recruit RNAi components to maintain heterochromatin through a local, self-reinforcing loop of small RNA production that is in many ways analogous to the Ping-Pong amplification loop for piRNAs. Yeast and fly systems differ in their strategies for producing complementary substrates. Where yeast and plants use RNA-dependent RNA polymerases to produce antisense repeat sequences, Drosophila and mammals encode them from piRNA loci.

The PIWI Subclass of Argonaute Proteins

As used herein, the “Piwi subclass of Argonaute proteins” include mammalian as well as insect proteins that are homologs or orthologs of the Drosophila melanogaster Piwi protein.

Cox et al. (Genes Dev. 12: 3715-3727, 1998, incorporated herein by reference) cloned and characterized the Drosophila piwi gene, and showed that it is essential for GSC maintenance in both males and females. The piwi protein is highly basic, especially in the C-terminal 100 amino acid residues, and is well conserved in evolution. Cox et al. (supra) also cloned 2 piwi-like genes in C. elegans that are required for GSC renewal, and also found sequence similarity with 2 Arabidopsis thaliana proteins required for meristem cell division. By use of an EST with sequence similarity to the Drosophila piwi gene to screen a human testis cDNA library, they further cloned the human homolog, PIWIL1. The deduced PIWIL1 protein shares 47.1% overall sequence identity, and 58.7% identity within the C terminus, with the Drosophila protein. Cox et al. (supra) found no piwi-related genes in the bacteria and yeast genomes, suggesting that piwi has a stem cell-related function only in multicellular organisms. Piwi and piwi-related proteins differ in the N terminus but show high homology in the C terminus where they all contain a conserved 43-amino acid domain, which the authors designated the PIWI box.

Thus in certain embodiments, the Piwi subclass of Argonaute proteins also include the conserved C-terminal domain of any of the art-recognized PIWI proteins, or fusion proteins comprising such conserved C-terminal domains.

By PCR of CD34-positive hematopoietic cells, followed by 5′-RACE of a testis cDNA library, Sharma et al. (Blood 97: 426-434, 2001, incorporated herein by reference) cloned PIWIL1, which they called HIWI. PCR analysis of adult and fetal tissues detected highest HIWI expression in adult testis, followed by adult and fetal kidney. Weaker expression was detected in all other fetal tissues examined and in adult prostate, ovary, small intestine, heart, brain, liver, skeletal muscle, kidney, and pancreas. Semiquantitative RT-PCR revealed HIWI expression in CD34-positive hematopoietic cells, and HIWI expression diminished during differentiation. HIWI was not expressed in C34-negative cells.

By 5′-RACE of testis mRNA, Qiao et al. (Oncogene 21: 3988-3999, 2002, incorporated herein by reference) obtained a full-length HIWI cDNA. The deduced 861-amino acid protein has a calculated molecular mass of 98.5 kD and contains a central PAZ motif and a C-terminal PIWI motif.

Deng and Lin (Dev. Cell 2: 819-830, 2002, incorporated herein by reference) cloned a mouse Piwi11 cDNA, which they called Miwi.

All these proteins are also within the scope of the subject Piwi subclass of Argonaute proteins. Protein sequences for these proteins include GenBank accession numbers: BAF49084, EAW98511, EAW98510, EAW98509, Q96J94, NP_{—}004755, BAC04068, AAH28581, AAC97371, AAK92281, AAK69348, etc. Polynucleotide sequences encoding these proteins include GenBank accession numbers: AB274731, CH471054, BC028581, AC127071, AK093133, AF104260, AF264004, AF387507, BG718140.

In certain embodiments, the subject Piwi subclass of Argonaute proteins may also include any polypeptides sharing at least 60%, 70%, 80%, 90%, 95%, 99% or more sequence identity to any of the above-referenced Piwi proteins, especially in the conserved C-terminal domain, which polypeptides preferably have one or more conserved functions of the naturally occurring Piwi proteins.

In certain embodiments, the subject Piwi subclass of Argonaute proteins may also include any polypeptides encoded by polynucleotides sharing at least 60%, 70%, 80%, 90%, 95%, 99% or more sequence identity to any of the above-referenced Piwi-encoding polynucleotides, and/or polynucleotides that hybridize under stringent conditions to any of the above-referenced Piwi-encoding polynucleotides. Preferably, the encoded polypeptides have one or more conserved functions of the naturally occurring Piwi proteins.

The Aubergine Subclass of Argonaute Proteins

As used herein, the “Aubergine subclass of Argonaute proteins” include mammalian as well as insect proteins that are homologs or orthologs of the Drosophila melanogaster Aubergine protein.

Harris and McDonald (Development 128: 2823-2832, 2001, incorporated by reference) showed that the Drosophila gene sting (Schmidt et al., Genetics 151: 749-760, 1999), a member of an ancient gene family that includes the gene for the eukaryotic translation initiation factor eIF2C (Zou et al., Gene 211: 187-194, 1998), is the same gene as aubergine. They also identified four other members of the eIF2C-like gene family in the Drosophila genome. One of these is piwi (Cox et al., supra). Two additional members, CG7439 and dAGO1, are reported in the genome annotation (Adams et al., Science 287: 2185-2195, 2000, incorporated by reference). The latter is the closest known relative of eIF2C in flies and is presumably the Drosophila eIF2C homolog. The authors also identified a fifth family member, corresponding to the genomic sequence AE003107 (Adams et al., supra) and EST clot 2083 (Rubin et al., Science 287: 2222-2224, 2000, incorporated by reference), by tBLASTn searches of the BDGP databases using parts of Aub protein as the query sequence.

The central and C-terminal portions of Aub contain two conserved regions, designated the PAZ and Piwi domains (Cerutti et al., Trends Biochem. Sci. 25: 481-482, 2000), which are encoded by a group of genes from organisms as diverse as plants, fungi and metazoans (including vertebrates). Recently, several of these genes have been characterized genetically and have been found to play essential roles in development. Both argonaute (ago1) and pinhead/zwille are required for maintenance of the axillary shoot meristem in Arabidopsis thaliana (Bohmert et al., 1998; Moussian et al., 1998; Lynn et al., 1999). In Drosophila, piwi has a demonstrated role in germline stem cell maintenance (Cox et al., 1998; Cox et al., 2000). Similarly, two Caenorhabditis elegans genes closely related to aub and piwi, prg-1 and prg-2, are also likely to be involved in germline proliferation (Cox et al., 1998). Other genes in the eIF2C/piwi family are implicated in mediating double-stranded RNA interference (RNAi) in C. elegans (rde-1; Tabara et al., 1999; Grishok et al., 2000) or the potentially related phenomena of post transcriptional gene silencing (PTGS) in Arabidopsis (ago1; Fagard et al., 2000) and quelling in Neurospora (qde-2; Catalanotto et al., 2000). The roles for ago1 in both PTGS and a cell fate decision reveal that a single gene in the family can carry out two functions, but it is not known if these functions are mechanistically distinct.

Thus in certain embodiments, the Aubergine subclass of Argonaute proteins also include bioactive fragments with the conserved PAZ and Piwi domains of any of the art-recognized Anbergine proteins, or fusion proteins comprising such conserved domains.

At least one specific biochemical activity has been demonstrated for one gene product in the family, the translation initiation factor eIF2C (formerly Co-eIF-2A) (Zou et al., supra). eIF2C purified from rabbit reticulocytes has two related activities that affect the ternary complex, which is composed of initiator methionine tRNA, GTP and eIF-2. The ternary complex binds the 40S ribosomal subunit to allow scanning for AUG codons in mRNA (for a review, see Hinnebusch, In Translational Control of Gene Exression (ed. N. Sonenberg, J. W. B. Hershey and M. B. Matthews), pp. 185-243. Cold Spring Harbor, N.Y.: Cold Spring Harbor Laboratory Press, 2000). Purified eIF2C stimulates formation of the ternary complex from components present at physiological levels, and it stabilizes the complex against dissociation in the presence of natural mRNAs.

Wild-type sequence for the Drosophila aubergine has the GenBank Accession Number X94613 and AAD38655. Other sequences are disclosed in the cited references, and are hereby incorporated by reference.

In certain embodiments, the subject Aubergine subclass of Argonaute proteins may also include any polypeptides sharing at least 60%, 70%, 80%, 90%, 95%, 99% or more sequence identity to any of the above-referenced Aubergine proteins, especially in the conserved PAZ and Piwi domains, which polypeptides preferably have one or more conserved functions of the naturally occurring Aubergine proteins.

In certain embodiments, the subject Aubergine subclass of Argonaute proteins may also include any polypeptides encoded by polynucleotides sharing at least 60%, 70%, 80%, 90%, 95%, 99% or more sequence identity to any of the above-referenced Aubergine-encoding polynucleotides, and/or polynucleotides that hybridize under stringent conditions to any of the above-referenced Aubergine-encoding polynucleotides. Preferably, the encoded polypeptides have one or more conserved functions of the naturally occurring Aubergine proteins.

The Ago3 Subclass of Argonaute Proteins

As used herein, the “Ago3 subclass of Argonaute proteins” include mammalian as well as insect proteins that are homologs or orthologs of the Drosophila melanogaster Ago3 protein.

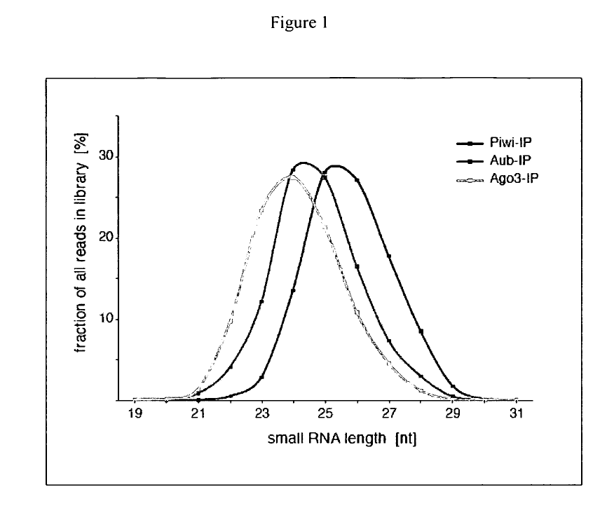

A phylogenetic tree of the Argonaute proteins is provided in the review article by Carmell et al. (Genes Dev. 16(21): 2733-42, 2002, the article and the sequences referred-to therein are all incorporated by reference). In FIG. 1 of Carmell, Ago subfamily is indicated in red, Piwi subfamily is in blue, orphans are in black. Accession nos. are: NP_{—}510322, ALG-1; NP_{—}493837, ALG-2; AAD40098, ZWILLE; AAD38655, aubergine/sting; JC6569, rabbit eIF-2C; CAA98113, Prg-1; AAB37734, Prg-2; AAF06159, RDE-1; AAF43641, QDE2; AAC18440, AGO1; NP_{—}523734, dAgo1; NP_{—}476875, piwi; AAF49619 plus additional N-terminal sequence from Hammond et al. (Science 293: 1146-1150, 2001), dAgo2; T41568, SPCC736.11; AY135687, mAgo1; AY135688, mAgo2; AY135689, mAgo3; AY135690, mAgo4; AY135691, mAgo5; AY135692, Miwi2; NP_{—}067283, MILI; NP_{—}067286, MIWI; XP_{—}050334, hAgo2/EIF2C2; XP_{—}029051, hAgo3; XP_{—}029053, hAgo1/EIF2C1; BAB13393, hAgo4; AAH25995, HILI; AAK92281, HIWI; and AAH31060, Hiwi2.

The International Radiation Hybrid Mapping Consortium mapped the AGO3 gene to human chromosome 1 (stSG53925). Carmell et al. (supra) stated that the AGO3 gene resides in tandem with the AGO1 (EIF2C1) and AGO4 genes on chromosome 1p35-p34. The orthologous genes in mouse are in the same orientation on chromosome 4.

### 3. Polynucleotide Modifications

In certain embodiments, the subject piRNA polynucleotides may be modified at various locations, including the sugar moiety, the phosphodiester linkage, and/or the base.

Sugar moieties include natural, unmodified sugars, e.g., monosaccharide (such as pentose, e.g., ribose, deoxyribose), modified sugars and sugar analogs. In general, possible modifications of polynucleotides, particularly of a sugar moiety, include, for example, replacement of one or more of the hydroxyl groups with a halogen, a heteroatom, an aliphatic group, or the functionalization of the hydroxyl group as an ether, an amine, a thiol, or the like.

One particularly useful group of modified polynucleotides are 2′-O-methyl nucleotides. Such 2′-O-methyl nucleotides may be referred to as “methylated,” and the corresponding nucleotides may be made from unmethylated nucleotides followed by alkylation or directly from methylated nucleotide reagents. Modified polynucleotides may be used in combination with unmodified polynucleotides. For example, an oligonucleotide of the invention may contain both methylated and unmethylated polynucleotides.

Some exemplary modified polynucleotides include sugar- or backbone-modified ribonucleotides. Modified ribonucleotides may contain a nonnaturally occurring base (instead of a naturally occurring base), such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine. Also, sugar-modified ribonucleotides may have the 2′-OH group replaced by a H, alxoxy (or OR), R or alkyl, halogen, SH, SR, amino (such as NH₂, NHR, NR₂), or CN group, wherein R is lower alkyl, alkenyl, or alkynyl.

Exemplary modifications on nucleosides may comprise one or more of: 2′-methoxyethoxy, 2′-methyl-thio-ethyl, 2′-deoxy-2′-fluoro, 2′-deoxy-2′-chloro, 2-azido, 2′-O-trifluoromethyl, 2′-O-ethyl-trifluoromethoxy, 2′-O-difluoromethoxy-ethoxy, 4′-thio, or 2′-O-methyl modifications, or mixtures thereof.

Modified ribonucleotides may also have the phosphoester group connecting to adjacent ribonucleotides replaced by a modified group, e.g., of phosphothioate group. More generally, the various nucleotide modifications may be combined.

Exemplary modifications on phosphate-sugar backbone comprise phosphorothioate, phosphoramidate, phosphodithioates, or chimeric methylphosphonate-phosphodiester linkages.

To further maximize endo- and exo-nuclease resistance, in addition to the use of 2′-modified polynucleotides in the ends, inter-polynucleotide linkages other than phosphodiesters may be used. For example, such end blocks may be used alone or in conjunction with phosphothioate linkages between the 2′-O-methly linkages. Preferred 2′-modified nucleotides are 2′-modified end nucleotides.

Although the piRNA may be substantially identical to at least a portion of the target gene (or genes), at least with respect to the base pairing properties, the sequence need not be perfectly identical to be useful, e.g., to inhibit expression of a target gene's phenotype. In certain embodiments, higher homology can be used to compensate for the use of a shorter piRNA. In some cases, the piRNA sequence generally will be substantially identical (although in antisense orientation) or complementary to the target gene sequence.

The use of 2′-O-methyl RNA may also be beneficially in circumstances in which it is desirable to minimize cellular stress responses. RNA having 2′-O-methyl polynucleotides may not be recognized by cellular machinery that is thought to recognize unmodified RNA.

Overall, modified sugars may include D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano and the like. In one embodiment, the sugar moiety can be a hexose and incorporated into an oligonucleotide as described (Augustyns, K., et al., Nucl. Acids. Res. 18:4711 (1992)). Exemplary polynucleotides can be found, e.g., in U.S. Pat. No. 5,849,902, incorporated by reference herein.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, unless otherwise specified, the term alkyl includes both “unsubstituted alkyls” and “substituted alkyls,” the latter of which refers to alkyl moieties having independently selected substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl(benzyl)). The term “alkyl” also includes the side chains of natural and unnatural amino acids. The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl(alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, unless otherwise specified, the term alkenyl includes both “unsubstituted alkenyls” and “substituted alkenyls,” the latter of which refers to alkenyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acyl amino (including alkylcarbonylamino, arylcarbonyl amino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, unless otherwise specified, the term alkynyl includes both “unsubstituted alkynyls” and “substituted alkynyls,” the latter of which refers to alkynyl moieties having independently selected substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups. The alkoxy groups can be substituted with independently selected groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulffiydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “heteroatom” includes atoms of any element other than carbon or hydrogen. Preferred heteroatoms are nitrogen, oxygen, sulfur and phosphorus.

The term “hydroxy” or “hydroxyl” includes groups with an —OH or —O— (with an appropriate counterion).

The term “halogen” includes fluorine, bromine, chlorine, iodine, etc. The term “perhalogenated” generally refers to a moiety wherein all hydrogens are replaced by halogen atoms.

The term “substituted” includes independently selected substituents which can be placed on the moiety and which allow the molecule to perform its intended function. Examples of substituents include alkyl, alkenyl, alkynyl, aryl, (CR′R″)₀₋₃NR′R″, (CR′R″)₀₋₃CN, NO₂, halogen, (CR′R″)₀₋₃C(halogen)₃, (CR′R″)₀₋₃CH(halogen)₂, (CR′R″)₀₋₃CH₂(halogen), (CR′R″)₀₋₃CONR′R″, (CR′R″)₀₋₃S(O)₁₋₂NR′R″, (CR′R″)₀₋₃CHO, (CR′R″)₀₋₃(CR′R″)₀₋₃H, (CR′R″)₀₋₃S(O)₀₋₂R′, (CR′R″)₀₋₃O(CR′R″)₀₋₃H, (CR′R″)₀₋₃COR′, (CR′R″)₀₋₃CO₂R′, or (CR′R″)₀₋₃OR′ groups; wherein each R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂—O—(CH₂)₂— group.

The term “amine” or “amino” includes compounds or moieties in which a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups.

The term “ether” includes compounds or moieties which contain an oxygen bonded to two different carbon atoms or heteroatoms. For example, the term includes “alkoxyalkyl,” which refers to an alkyl, alkenyl, or alkynyl group covalently bonded to an oxygen atom which is covalently bonded to another alkyl group.

The term “base” includes the known purine and pyrimidine heterocyclic bases, deazapurines, and analogs (including heterocyclic substituted analogs, e.g., aminoethyoxy phenoxazine), derivatives (e.g., 1-alkyl-, 1-alkenyl-, heteroaromatic- and 1-alkynyl derivatives) and tautomers thereof. Examples of purines include adenine, guanine, inosine, diaminopurine, and xanthine and analogs (e.g., 8-oxo-N⁶-methyladenine or 7-diazaxanthine) and derivatives thereof. Pyrimidines include, for example, thymine, uracil, and cytosine, and their analogs (e.g., 5-methylcytosine, 5-methyluracil, 5-(1-propynyl)uracil, 5-(1-propynyl)cytosine and 4,4-ethanocytosine). Other examples of suitable bases include non-purinyl and non-pyrimidinyl bases such as 2-aminopyridine and triazines.

In a preferred embodiment, the polynucleotides of the invention are RNA nucleotides. In another preferred embodiment, the polynucleotide of the invention are modified RNA nucleotides.

The term “nucleoside” includes bases which are covalently attached to a sugar moiety, preferably ribose or deoxyribose. Examples of preferred nucleosides include ribonucleosides and deoxyribonucleosides. Nucleosides also include bases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2^{nd} Ed., Wiley-Interscience, New York, 1999).

The term “nucleotide” includes nucleosides which further comprise a phosphate group or a phosphate analog.

As used herein, the term “linkage” includes a naturally occurring, unmodified phosphodiester moiety (—O—(PO²⁻)—O—) that covalently couples adjacent nucleotides. As used herein, the term “substitute linkage” includes any analog or derivative of the native phosphodiester group that covalently couples adjacent nucleotides. Substitute linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Such substitute linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothiate linkages.

In certain embodiments, oligonucleotides of the invention comprise 3′ and 5′ termini (except for circular oligonucleotides). In one embodiment, the 3′ and 5′ termini of an oligonucleotide can be substantially protected from nucleases e.g., by modifying the 3′ or 5′ linkages (e.g., U.S. Pat. No. 5,849,902 and WO 98/13526). For example, oligonucleotides can be made resistant by the inclusion of a “blocking group.” The term “blocking group” or “terminal cap moiety” as used herein refers to substituents (e.g., other than OH groups) that can be attached to oligonucleotides, either as protecting groups or coupling groups for synthesis (e.g., FITC, propyl(CH₂—CH₂—CH₃), glycol (—O—CH₂—CH₂—O—) phosphate (PO₃²⁻), hydrogen phosphonate, or phosphoramidite). “Blocking groups” pr “terminal cap moiety” also include “end blocking groups” or “exonuclease blocking groups” which protect the 5′ and 3′ termini of the oligonucleotide, including modified nucleotides and non-nucleotide exonuclease resistant structures.

Exemplary end-blocking groups include cap structures (e.g., a 7-methylguanosine cap), inverted nucleotides, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates) and the like. The 3′ terminal nucleotide can comprise a modified sugar moiety. The 3′ terminal nucleotide comprises a 3′-O that can optionally be substituted by a blocking group that prevents 3′-exonuclease degradation of the oligonucleotide. For example, the 3′-hydroxyl can be esterified to a nucleotide through a 3′→3′ internucleotide linkage. For example, the alkyloxy radical can be methoxy, ethoxy, or isopropoxy, and preferably, ethoxy. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

piRNA sequences of the present invention may include “morpholino oligonucleotides.” Morpholino oligonucleotides are non-ionic and function by an RNase H-independent mechanism. Each of the 4 genetic bases (Adenine, Cytosine, Guanine, and Thymine/Uracil) of the morpholino oligonucleotides is linked to a 6-membered morpholine ring. Morpholino oligonucleotides are made by joining the 4 different subunit types by, e.g., non-ionic phosphorodiamidate inter-subunit linkages. Morpholino oligonucleotides have many advantages including: complete resistance to nucleases (Antisense & Nucl. Acid Drug Dev. 1996. 6:267); predictable targeting (Biochemica Biophysica Acta. 1999. 1489:141); reliable activity in cells (Antisense & Nucl. Acid Drug Dev. 1997. 7:63); excellent sequence specificity (Antisense & Nucl. Acid Drug Dev. 1997. 7:151); minimal non-antisense activity (Biochemica Biophysica Acta. 1999. 1489:141); and simple osmotic or scrape delivery (Antisense & Nucl. Acid Drug Dev. 1997. 7:291). Morpholino oligonucleotides are also preferred because of their non-toxicity at high doses. A discussion of the preparation of morpholino oligonucleotides can be found in Antisense & Nucl. Acid Drug Dev. 1997. 7:187.

### 4. Synthesis

piRNA of the invention can be synthesized by any method known in the art, e.g., using enzymatic synthesis and/or chemical synthesis. The oligonucleotides can be synthesized in vitro (e.g., using enzymatic synthesis and chemical synthesis) or in vivo (using recombinant DNA technology well known in the art).

In a preferred embodiment, chemical synthesis is used for modified polynucleotides. Chemical synthesis of linear oligonucleotides is well known in the art and can be achieved by solution or solid phase techniques. Preferably, synthesis is by solid phase methods. Oligonucleotides can be made by any of several different synthetic procedures including the phosphoramidite, phosphite triester, H-phosphonate, and phosphotriester methods, typically by automated synthesis methods.

Oligonucleotide synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; and U.S. Pat. No. 5,264,423.

The synthesis method selected can depend on the length of the desired oligonucleotide and such choice is within the skill of the ordinary artisan. For example, the phosphoramidite and phosphite triester method can produce oligonucleotides having 175 or more nucleotides, while the H-phosphonate method works well for oligonucleotides of less than 100 nucleotides. If modified bases are incorporated into the oligonucleotide, and particularly if modified phosphodiester linkages are used, then the synthetic procedures are altered as needed according to known procedures. In this regard, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984). Moreover, linear oligonucleotides of defined sequence, including some sequences with modified nucleotides, are readily available from several commercial sources.

The oligonucleotides may be purified by polyacrylamide gel electrophoresis, or by any of a number of chromatographic methods, including gel chromatography and high pressure liquid chromatography. To confirm a nucleotide sequence, especially unmodified nucleotide sequences, oligonucleotides may be subjected to DNA sequencing by any of the known procedures, including Maxam and Gilbert sequencing, Sanger sequencing, capillary electrophoresis sequencing, the wandering spot sequencing procedure or by using selective chemical degradation of oligonucleotides bound to Hybond paper. Sequences of short oligonucleotides can also be analyzed by laser desorption mass spectroscopy or by fast atom bombardment (McNeal, et al., 1982, J. Am. Chem. Soc. 104:976; Viari, et al., 1987, Biomed. Environ. Mass Spectrom. 14:83; Grotjahn et al., 1982, Nuc. Acid Res. 10:4671). Sequencing methods are also available for RNA oligonucleotides.

The quality of oligonucleotides synthesized can be verified by testing the oligonucleotide by capillary electrophoresis and denaturing strong anion HPLC (SAX-HPLC) using, e.g., the method of Bergot and Egan. 1992. J. Chrom. 599:35.

Other exemplary synthesis techniques are well known in the art (see, e.g., Sambrook et al., Molecular Cloning: a Laboratory Manual, Second Edition (1989); DNA Cloning, Volumes I and II (D N Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

In certain embodiments, the subject piRNA constructs or at least portions thereof are transcribed from expression vectors encoding the subject constructs. Any art recognized vectors may be use for this purpose. The transcribed piRNA constructs may be isolated and purified, before desired modifications (such as replacing an unmodified sense strand with a modified one, etc.) are carried out.

### 5. Delivery/Carrier

### Uptake of Oligonucleotides by Cells

The subject piRNA oligonucleotides and oligonucleotide compositions are contacted with (i.e., brought into contact with, also referred to herein as administered or delivered to) and taken up by one or more cells or a cell lysate. The term “cells” includes prokaryotic and eukaryotic cells, preferably vertebrate cells, and, more preferably, mammalian cells. In a preferred embodiment, the oligonucleotide compositions of the invention are contacted with human cells.

Oligonucleotide compositions of the invention can be contacted with cells in vitro, e.g., in a test tube or culture dish, (and may or may not be introduced into a subject) or in vivo, e.g., in a subject such as a mammalian subject. Oligonucleotides are taken up by cells at a slow rate by endocytosis, but endocytosed oligonucleotides are generally sequestered and not available, e.g., for hybridization to a target nucleic acid molecule. In one embodiment, cellular uptake can be facilitated by electroporation or calcium phosphate precipitation. However, these procedures are only useful for in vitro or ex vivo embodiments, are not convenient and, in some cases, are associated with cell toxicity.

In another embodiment, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of oligonucleotides can also be mediated by the use of vectors (See e.g., Shi, Y. 2003. Trends Genet. 2003 Jan. 19:9; Reichhart J M et al. Genesis. 2002. 34(1-2):1604, Yu et al. 2002. Proc. Natl. Acad. Sci. USA 99:6047; Sui et al. 2002. Proc. Natl. Acad. Sci. USA 99:5515) viruses, polyamine or polycation conjugates using compounds such as polylysine, protamine, or Ni, N12-bis(ethyl) spermine (see, e.g., Bartzatt, R. et al. 1989. Biotechnol. Appl. Biochem. 11:133; Wagner E. et al. 1992. Proc. Natl. Acad. Sci. 88:4255).

The optimal protocol for uptake of oligonucleotides will depend upon a number of factors, the most crucial being the type of cells that are being used. Other factors that are important in uptake include, but are not limited to, the nature and concentration of the oligonucleotide, the confluence of the cells, the type of culture the cells are in (e.g., a suspension culture or plated) and the type of media in which the cells are grown.

### Conjugating Agents

Conjugating agents bind to the oligonucleotide in a covalent manner. In one embodiment, oligonucleotides can be derivatized or chemically modified by binding to a conjugating agent to facilitate cellular uptake. For example, covalent linkage of a cholesterol moiety to an oligonucleotide can improve cellular uptake by 5- to 10-fold which in turn improves DNA binding by about 10-fold (Boutorin et al., 1989, FEBS Letters 254:129-132). Conjugation of octyl, dodecyl, and octadecyl residues enhances cellular uptake by 3-, 4-, and 10-fold as compared to unmodified oligonucleotides (Vlassov et al., 1994, Biochimica et Biophysica Acta 1197:95-108). Similarly, derivatization of oligonucleotides with poly-L-lysine can aid oligonucleotide uptake by cells (Schell, 1974, Biochem. Biophys. Acta 340:323, and Lemaitre et al., 1987, Proc. Natl. Acad. Sci. USA 84:648).

Certain protein carriers can also facilitate cellular uptake of oligonucleotides, including, for example, serum albumin, nuclear proteins possessing signals for transport to the nucleus, and viral or bacterial proteins capable of cell membrane penetration. Therefore, protein carriers are useful when associated with or linked to the oligonucleotides. Accordingly, the present invention provides for derivatization of oligonucleotides with groups capable of facilitating cellular uptake, including hydrocarbons and non-polar groups, cholesterol, long chain alcohols (i.e., hexanol), poly-L-lysine and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes, and steroids. A major advantage of using conjugating agents is to increase the initial membrane interaction that leads to a greater cellular accumulation of oligonucleotides.

Certain conjugating agents that may be used with the instant constructs include those described in WO04048545A2 and US20040204377A1 (all incorporated herein by their entireties), such as a Tat peptide, a sequence substantially similar to the sequence of SEQ ID NO: 12 of WO04048545A2 and US20040204377A1, a homeobox (hox) peptide, a MTS, VP22, MPG, at least one dendrimer (such as PAMAM), etc.

Other conjugating agents that may be used with the instant constructs include those described in WO07089607A2 (incorporated herein), which describes various nanotransporters and delivery complexes for use in delivery of nucleic acid molecules and/or other pharmaceutical agents in vivo and in vitro. Using such delivery complexes, the subject piRNAs can be delivered while conjugated or associated with a nanotransporter comprising a core conjugated with at least one functional surface group. The core may be a nanoparticle, such as a dendrimer (e.g., a polylysine dendrimer). The core may also be a nanotube, such as a single walled nanotube or a multi-walled nanotube. The functional surface group is at least one of a lipid, a cell type specific targeting moiety, a fluorescent molecule, and a charge controlling molecule. For example, the targeting moiety may be a tissue-selective peptide. The lipid may be an oleoyl lipid or derivative thereof. Exemplary nanotransporter include NOP-7 or HBOLD.

### Encapsulating Agents

Encapsulating agents entrap oligonucleotides within vesicles. In another embodiment of the invention, an oligonucleotide may be associated with a carrier or vehicle, e.g., liposomes or micelles, although other carriers could be used, as would be appreciated by one skilled in the art. Liposomes are vesicles made of a lipid bilayer having a structure similar to biological membranes. Such carriers are used to facilitate the cellular uptake or targeting of the oligonucleotide, or improve the oligonucleotide's pharmacokinetic or toxicologic properties.

For example, the oligonucleotides of the present invention may also be administered encapsulated in liposomes, pharmaceutical compositions wherein the active ingredient is contained either dispersed or variously present in corpuscles consisting of aqueous concentric layers adherent to lipidic layers. The oligonucleotides, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. The diameters of the liposomes generally range from about 15 nm to about 5 microns.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes include the following: they are non-toxic and biodegradable in composition; they display long circulation half-lives; and recognition molecules can be readily attached to their surface for targeting to tissues. Finally, cost-effective manufacture of liposome-based pharmaceuticals, either in a liquid suspension or lyophilized product, has demonstrated the viability of this technology as an acceptable drug delivery system.

### Complexing Agents

Complexing agents bind to the oligonucleotides of the invention by a strong but non-covalent attraction (e.g., an electrostatic, van der Waals, pi-stacking, etc. interaction). In one embodiment, oligonucleotides of the invention can be complexed with a complexing agent to increase cellular uptake of oligonucleotides. An example of a complexing agent includes cationic lipids. Cationic lipids can be used to deliver oligonucleotides to cells.

The term “cationic lipid” includes lipids and synthetic lipids having both polar and non-polar domains and which are capable of being positively charged at or around physiological pH and which bind to polyanions, such as nucleic acids, and facilitate the delivery of nucleic acids into cells. In general cationic lipids include saturated and unsaturated alkyl and alicyclic ethers and esters of amines, amides, or derivatives thereof. Straight-chain and branched alkyl and alkenyl groups of cationic lipids can contain, e.g., from 1 to about 25 carbon atoms. Preferred straight chain or branched alkyl or alkene groups have six or more carbon atoms. Alicyclic groups include cholesterol and other steroid groups. Cationic lipids can be prepared with a variety of counterions (anions) including, e.g., Cl⁻, Br⁻, I⁻, F⁻, acetate, trifluoroacetate, sulfate, nitrite, and nitrate.

Examples of cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl]cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphothioate oligonucleotide. (Vlassov et al., 1994, Biochimica et Biophysica Acta 1197:95-108). Oligonucleotides can also be complexed with, e.g., poly (L-lysine) or avidin and lipids may, or may not, be included in this mixture, e.g., steryl-poly (L-lysine).

Cationic lipids have been used in the art to deliver oligonucleotides to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). Other lipid compositions which can be used to facilitate uptake of the instant oligonucleotides can be used in connection with the claimed methods. In addition to those listed supra, other lipid compositions are also known in the art and include, e.g., those taught in U.S. Pat. No. 4,235,871; U.S. Pat. Nos. 4,501,728; 4,837,028; 4,737,323.

In one embodiment lipid compositions can further comprise agents, e.g., viral proteins to enhance lipid-mediated transfections of oligonucleotides (Kamata, et al., 1994. Nucl. Acids. Res. 22:536). In another embodiment, oligonucleotides are contacted with cells as part of a composition comprising an oligonucleotide, a peptide, and a lipid as taught, e.g., in U.S. Pat. No. 5,736,392. Improved lipids have also been described which are serum resistant (Lewis, et al., 1996. Proc. Natl. Acad. Sci. 93: 3176). Cationic lipids and other complexing agents act to increase the number of oligonucleotides carried into the cell through endocytosis.

In another embodiment N-substituted glycine oligonucleotides (peptoids) can be used to optimize uptake of oligonucleotides. Peptoids have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114: 10646; Zuckermann, R. N., et al. 1992. Int. J. Peptide Protein Res. 40:497). Combinations of cationic lipids and peptoids, liptoids, can also be used to optimize uptake of the subject oligonucleotides (Hunag, et al., 1998. Chemistry and Biology. 5:345). Liptoids can be synthesized by elaborating peptoid oligonucleotides and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. USA. 93:3176). In one embodiment, a composition for delivering oligonucleotides of the invention comprises a number of arginine, lysine, histidine or ornithine residues linked to a lipophilic moiety (see e.g., U.S. Pat. No. 5,777,153).

In another embodiment, a composition for delivering oligonucleotides of the invention comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine. Preferably a preponderance of neutral amino acids with long neutral side chains are used.

In one embodiment, the cells to be contacted with an oligonucleotide composition of the invention are contacted with a mixture comprising the oligonucleotide and a mixture comprising a lipid, e.g., one of the lipids or lipid compositions described supra for between about 12 hours to about 24 hours. In another embodiment, the cells to be contacted with an oligonucleotide composition are contacted with a mixture comprising the oligonucleotide and a mixture comprising a lipid, e.g., one of the lipids or lipid compositions described supra for between about 1 and about five days. In one embodiment, the cells are contacted with a mixture comprising a lipid and the oligonucleotide for between about three days to as long as about 30 days. In another embodiment, a mixture comprising a lipid is left in contact with the cells for at least about five to about 20 days. In another embodiment, a mixture comprising a lipid is left in contact with the cells for at least about seven to about 15 days.

For example, in one embodiment, an oligonucleotide composition can be contacted with cells in the presence of a lipid such as cytofectin CS or GSV (available from Glen Research; Sterling, Va.), GS3815, GS2888 for prolonged incubation periods as described herein.

In one embodiment, the incubation of the cells with the mixture comprising a lipid and an oligonucleotide composition does not reduce the viability of the cells. Preferably, after the transfection period the cells are substantially viable. In one embodiment, after transfection, the cells are between at least about 70% and at least about 100% viable. In another embodiment, the cells are between at least about 80% and at least about 95% viable. In yet another embodiment, the cells are between at least about 85% and at least about 90% viable.

In one embodiment, oligonucleotides are modified by attaching a peptide sequence that transports the oligonucleotide into a cell, referred to herein as a “transporting peptide.” In one embodiment, the composition includes an oligonucleotide which is complementary to a target nucleic acid molecule encoding the protein, and a covalently attached transporting peptide.

The language “transporting peptide” includes an amino acid sequence that facilitates the transport of an oligonucleotide into a cell. Exemplary peptides which facilitate the transport of the moieties to which they are linked into cells are known in the art, and include, e.g., HIV TAT transcription factor, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88: 223).

Oligonucleotides can be attached to the transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272: 16010). For example, in one embodiment, oligonucleotides bearing an activated thiol group are linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8: 84; Prochiantz. 1996. Current Opinion in Neurobiol. 6: 629; Allinquant et al. 1995. J. Cell Biol. 128:919). In another embodiment, a Boc-Cys-(Npys)OH group can be coupled to the transport peptide as the last (N-terminal) amino acid and an oligonucleotide bearing an SH group can be coupled to the peptide (Troy et al. 1996. J. Neurosci. 16:253).

In one embodiment, a linking group can be attached to a nucleotide and the transporting peptide can be covalently attached to the linker. In one embodiment, a linker can function as both an attachment site for a transporting peptide and can provide stability against nucleases. Examples of suitable linkers include substituted or unsubstituted C₁-C₂₀ alkyl chains, C₂-C₂₀ alkenyl chains, C₂-C₂₀ alkynyl chains, peptides, and heteroatoms (e.g., S, O, NH, etc.). Other exemplary linkers include bifunctional crosslinking agents such as sulfosuccinimidyl-4-(maleimidophenyl)-butyrate (SMPB) (see, e.g., Smith et al. Biochem J 1991.276: 417-2).

In one embodiment, oligonucleotides of the invention are synthesized as molecular conjugates which utilize receptor-mediated endocytotic mechanisms for delivering genes into cells (see, e.g., Bunnell et al. 1992. Somatic Cell and Molecular Genetics. 18:559, and the references cited therein).

### Targeting Agents

The delivery of oligonucleotides can also be improved by targeting the oligonucleotides to a cellular receptor. The targeting moieties can be conjugated to the oligonucleotides or attached to a carrier group (i.e., poly(L-lysine) or liposomes) linked to the oligonucleotides. This method is well suited to cells that display specific receptor-mediated endocytosis.

For instance, oligonucleotide conjugates to 6-phosphomannosylated proteins are internalized 20-fold more efficiently by cells expressing mannose 6-phosphate specific receptors than free oligonucleotides. The oligonucleotides may also be coupled to a ligand for a cellular receptor using a biodegradable linker. In another example, the delivery construct is mannosylated streptavidin which forms a tight complex with biotinylated oligonucleotides. Mannosylated streptavidin was found to increase 20-fold the internalization of biotinylated oligonucleotides. (Vlassov et al. 1994. Biochimica et Biophysica Acta 1197:95-108).

In addition specific ligands can be conjugated to the polylysine component of polylysine-based delivery systems. For example, transferrin-polylysine, adenovirus-polylysine, and influenza virus hemagglutinin HA-2 N-terminal fusogenic peptides-polylysine conjugates greatly enhance receptor-mediated DNA delivery in eucaryotic cells. Mannosylated glycoprotein conjugated to poly(L-lysine) in aveolar macrophages has been employed to enhance the cellular uptake of oligonucleotides. Liang et al. 1999. Pharmazie 54:559-566.

Because malignant cells have an increased need for essential nutrients such as folic acid and transferrin, these nutrients can be used to target oligonucleotides to cancerous cells. For example, when folic acid is linked to poly(L-lysine) enhanced oligonucleotide uptake is seen in promyelocytic leukaemia (HL-60) cells and human melanoma (M−14) cells. Ginobbi et al. 1997. Anticancer Res. 17:29. In another example, liposomes coated with maleylated bovine serum albumin, folic acid, or ferric protoporphyrin IX, show enhanced cellular uptake of oligonucleotides in murine macrophages, KB cells, and 2.2.15 human hepatoma cells. Liang et al. 1999. Pharmazie 54:559-566.

Liposomes naturally accumulate in the liver, spleen, and reticuloendothelial system (so-called, passive targeting). By coupling liposomes to various ligands such as antibodies are protein A, they can be actively targeted to specific cell populations. For example, protein A-bearing liposomes may be pretreated with H-2K specific antibodies which are targeted to the mouse major histocompatibility complex-encoded H-2K protein expressed on L cells. (Vlassov et al. 1994. Biochimica et Biophysica Acta 1197:95-108).

### 6. Administration

The optimal course of administration or delivery of the oligonucleotides may vary depending upon the desired result and/or on the subject to be treated. As used herein “administration” refers to contacting cells with oligonucleotides and can be performed in vitro or in vivo. The dosage of oligonucleotides may be adjusted to optimally reduce expression of a protein translated from a target nucleic acid molecule, e.g., as measured by a readout of RNA stability or by a therapeutic response.

For example, expression of the protein encoded by the nucleic acid target can be measured to determine whether or not the dosage regimen needs to be adjusted accordingly. In addition, an increase or decrease in RNA or protein levels in a cell or produced by a cell can be measured using any art recognized technique. By determining whether transcription has been decreased, the effectiveness of the oligonucleotide in inducing the cleavage of a target RNA can be determined.

Any of the above-described oligonucleotide compositions can be used alone or in conjunction with a pharmaceutically acceptable carrier. As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Oligonucleotides may be incorporated into liposomes or liposomes modified with polyethylene glycol or admixed with cationic lipids for parenteral administration. Incorporation of additional substances into the liposome, for example, antibodies reactive against membrane proteins found on specific target cells, can help target the oligonucleotides to specific cell types.

Moreover, the present invention provides for administering the subject oligonucleotides with an osmotic pump providing continuous infusion of such oligonucleotides, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, e.g., parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, or dextran, optionally, the suspension may also contain stabilizers. The oligonucleotides of the invention can be formulated in liquid solutions, preferably in physiologically compatible buffers such as Hank's solution or Ringer's solution. In addition, the oligonucleotides may be formulated in solid form and redissolved or suspended immediately prior to use. Lyophilized forms are also included in the invention.

Pharmaceutical preparations for topical administration include transdermal patches, ointments, lotions, creams, gels, drops, sprays, suppositories, liquids and powders. In addition, conventional pharmaceutical carriers, aqueous, powder or oily bases, or thickeners may be used in pharmaceutical preparations for topical administration.

Pharmaceutical preparations for oral administration include powders or granules, suspensions or solutions in water or non-aqueous media, capsules, sachets or tablets. In addition, thickeners, flavoring agents, diluents, emulsifiers, dispersing aids, or binders may be used in pharmaceutical preparations for oral administration.

For transmucosal or transdermal administration, penetrants appropriate to the barrier to be permeated are used in the formulation. Such penetrants are known in the art, and include, for example, for transmucosal administration bile salts and fusidic acid derivatives, and detergents. Transmucosal administration may be through nasal sprays or using suppositories. For oral administration, the oligonucleotides are formulated into conventional oral administration forms such as capsules, tablets, and tonics. For topical administration, the oligonucleotides of the invention are formulated into ointments, salves, gels, or creams as known in the art.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The described oligonucleotides may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the oligonucleotide to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the oligonucleotide at the lymph node. The oligonucleotide can be modified to diffuse into the cell, or the liposome can directly participate in the delivery of either the unmodified or modified oligonucleotide into the cell.

The chosen method of delivery will result in entry into cells. Preferred delivery methods include liposomes (10-400 nm), hydrogels, controlled-release polymers, and other pharmaceutically applicable vehicles, and microinjection or electroporation (for ex vivo treatments).

The pharmaceutical preparations of the present invention may be prepared and formulated as emulsions. Emulsions are usually heterogeneous systems of one liquid dispersed in another in the form of droplets usually exceeding 0.1 μm in diameter. The emulsions of the present invention may contain excipients such as emulsifiers, stabilizers, dyes, fats, oils, waxes, fatty acids, fatty alcohols, fatty esters, humectants, hydrophilic colloids, preservatives, and anti-oxidants may also be present in emulsions as needed. These excipients may be present as a solution in either the aqueous phase, oily phase or itself as a separate phase.

Examples of naturally occurring emulsifiers that may be used in emulsion formulations of the present invention include lanolin, beeswax, phosphatides, lecithin and acacia. Finely divided solids have also been used as good emulsifiers especially in combination with surfactants and in viscous preparations. Examples of finely divided solids that may be used as emulsifiers include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

Examples of preservatives that may be included in the emulsion formulations include methyl paraben, propyl paraben, quaternary ammonium salts, benzalkonium chloride, esters of p-hydroxybenzoic acid, and boric acid. Examples of antioxidants that may be included in the emulsion formulations include free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of oligonucleotides are formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system.

Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₅-C₁₀ glycerides, vegetable oils and silicone oil.

Microemulsions are particularly of interest from the standpoint of drug solubilization and the enhanced absorption of drugs. Lipid based microemulsions (both oil/water and water/oil) have been proposed to enhance the oral bioavailability of drugs.

Microemulsions offer improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11:1385; Ho et al., J. Pharm. Sci., 1996, 85:138-143). Microemulsions have also been effective in the transdermal delivery of active components in both cosmetic and pharmaceutical applications. It is expected that the microemulsion compositions and formulations of the present invention will facilitate the increased systemic absorption of oligonucleotides from the gastrointestinal tract, as well as improve the local cellular uptake of oligonucleotides within the gastrointestinal tract, vagina, buccal cavity and other areas of administration.

In an embodiment, the present invention employs various penetration enhancers to affect the efficient delivery of nucleic acids, particularly oligonucleotides, to the skin of animals. Even non-lipophilic drugs may cross cell membranes if the membrane to be crossed is treated with a penetration enhancer. In addition to increasing the diffusion of non-lipophilic drugs across cell membranes, penetration enhancers also act to enhance the permeability of lipophilic drugs.

Five categories of penetration enhancers that may be used in the present invention include: surfactants, fatty acids, bile salts, chelating agents, and non-chelating non-surfactants. Other agents may be utilized to enhance the penetration of the administered oligonucleotides include: glycols such as ethylene glycol and propylene glycol, pyrrols such as 2-15 pyrrol, azones, and terpenes such as limonene, and menthone.

The oligonucleotides, especially in lipid formulations, can also be administered by coating a medical device, for example, a catheter, such as an angioplasty balloon catheter, with a cationic lipid formulation. Coating may be achieved, for example, by dipping the medical device into a lipid formulation or a mixture of a lipid formulation and a suitable solvent, for example, an aqueous-based buffer, an aqueous solvent, ethanol, methylene chloride, chloroform and the like. An amount of the formulation will naturally adhere to the surface of the device which is subsequently administered to a patient, as appropriate. Alternatively, a lyophilized mixture of a lipid formulation may be specifically bound to the surface of the device. Such binding techniques are described, for example, in K. Ishihara et al., Journal of Biomedical Materials Research, Vol. 27, pp. 1309-1314 (1993), the disclosures of which are incorporated herein by reference in their entirety.

The useful dosage to be administered and the particular mode of administration will vary depending upon such factors as the cell type, or for in vivo use, the age, weight and the particular animal and region thereof to be treated, the particular oligonucleotide and delivery method used, the therapeutic or diagnostic use contemplated, and the form of the formulation, for example, suspension, emulsion, micelle or liposome, as will be readily apparent to those skilled in the art. Typically, dosage is administered at lower levels and increased until the desired effect is achieved. When lipids are used to deliver the oligonucleotides, the amount of lipid compound that is administered can vary and generally depends upon the amount of oligonucleotide agent being administered. For example, the weight ratio of lipid compound to oligonucleotide agent is preferably from about 1:1 to about 15:1, with a weight ratio of about 5:1 to about 10:1 being more preferred. Generally, the amount of cationic lipid compound which is administered will vary from between about 0.1 milligram (mg) to about 1 gram (g). By way of general guidance, typically between about 0.1 mg and about 10 mg of the particular oligonucleotide agent, and about 1 mg to about 100 mg of the lipid compositions, each per kilogram of patient body weight, is administered, although higher and lower amounts can be used.

The agents of the invention are administered to subjects or contacted with cells in a biologically compatible form suitable for pharmaceutical administration. By “biologically compatible form suitable for administration” is meant that the oligonucleotide is administered in a form in which any toxic effects are outweighed by the therapeutic effects of the oligonucleotide. In one embodiment, oligonucleotides can be administered to subjects. Examples of subjects include mammals, e.g., humans and other primates; cows, pigs, horses, and farming (agricultural) animals; dogs, cats, and other domesticated pets; mice, rats, and transgenic non-human animals.

Administration of an active amount of an oligonucleotide of the present invention is defined as an amount effective, at dosages and for periods of time necessary to achieve the desired result. For example, an active amount of an oligonucleotide may vary according to factors such as the type of cell, the oligonucleotide used, and for in vivo uses the disease state, age, sex, and weight of the individual, and the ability of the oligonucleotide to elicit a desired response in the individual. Establishment of therapeutic levels of oligonucleotides within the cell is dependent upon the rates of uptake and efflux or degradation. Decreasing the degree of degradation prolongs the intracellular half-life of the oligonucleotide. Thus, chemically-modified oligonucleotides, e.g., with modification of the phosphate backbone, may require different dosing.

The exact dosage of an oligonucleotide and number of doses administered will depend upon the data generated experimentally and in clinical trials. Several factors such as the desired effect, the delivery vehicle, disease indication, and the route of administration, will affect the dosage. Dosages can be readily determined by one of ordinary skill in the art and formulated into the subject pharmaceutical compositions. Preferably, the duration of treatment will extend at least through the course of the disease symptoms.

Dosage regima may be adjusted to provide the optimum therapeutic response. For example, the oligonucleotide may be repeatedly administered, e.g., several doses may be administered daily or the dose may be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

### 7. Therapeutic Use

By inhibiting the expression of a gene, the oligonucleotide compositions of the present invention can be used to treat any disease involving the expression of a protein. Examples of diseases that can be treated by oligonucleotide compositions, just to illustrate, include: cancer, retinopathies, autoimmune diseases, inflammatory diseases (i.e., ICAM-1 related disorders, Psoriasis, Ulcerative Colitus, Crohn's disease), viral diseases (i.e., HIV, Hepatitis C), and cardiovascular diseases.

In one embodiment, in vitro treatment of cells with oligonucleotides can be used for ex vivo therapy of cells removed from a subject (e.g., for treatment of leukemia or viral infection) or for treatment of cells which did not originate in the subject, but are to be administered to the subject (e.g., to eliminate transplantation antigen expression on cells to be transplanted into a subject). In addition, in vitro treatment of cells can be used in non-therapeutic settings, e.g., to evaluate gene function, to study gene regulation and protein synthesis or to evaluate improvements made to oligonucleotides designed to modulate gene expression or protein synthesis. In vivo treatment of cells can be useful in certain clinical settings where it is desirable to inhibit the expression of a protein. There are numerous medical conditions for which such therapy is reported to be suitable (see, e.g., U.S. Pat. No. 5,830,653) as well as respiratory syncytial virus infection (WO 95/22,553) influenza virus (WO 94/23,028), and malignancies (WO 94/08,003). Other examples of clinical uses are reviewed, e.g., in Glaser. 1996. Genetic Engineering News 16:1. Exemplary targets for cleavage by oligonucleotides include, e.g., protein kinase Ca, ICAM-1, c-raf kinase, p53, c-myb, and the bcr/abl fusion gene found in chronic myelogenous leukemia.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No. 4,683,195; Nucleic Acid Hybridization (B. D. Hames & S. J. Higgins eds. (1984)); the treatise, Methods In Enzymology (Academic Press, Inc., N.Y.); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

### EXAMPLES

The invention now being generally described, it will be more readily understood by reference to the following examples, which are included merely for purposes of illustration of certain aspects and embodiments of the present invention, and are not intended to limit the invention.

In order to probe mechanisms of transposon control in Drosophila and to illuminate similarities and differences between Piwi protein function in flies and mammals, Applicants first undertook a detailed analysis of small RNAs associated with three members of the Piwi clade in the Drosophila female germline. The results are presented in Examples I-VI below. These results indicate that the three Drosophila Piwi family members function in a transposon surveillance pathway that not only preserves a genetic memory of transposon exposure but also has the potential to adapt its response upon contact with dispersed and potentially active transposon copies.

### Example I

### Piwi Family Members Have Distinct Expression Patterns in Drosophila Ovaries

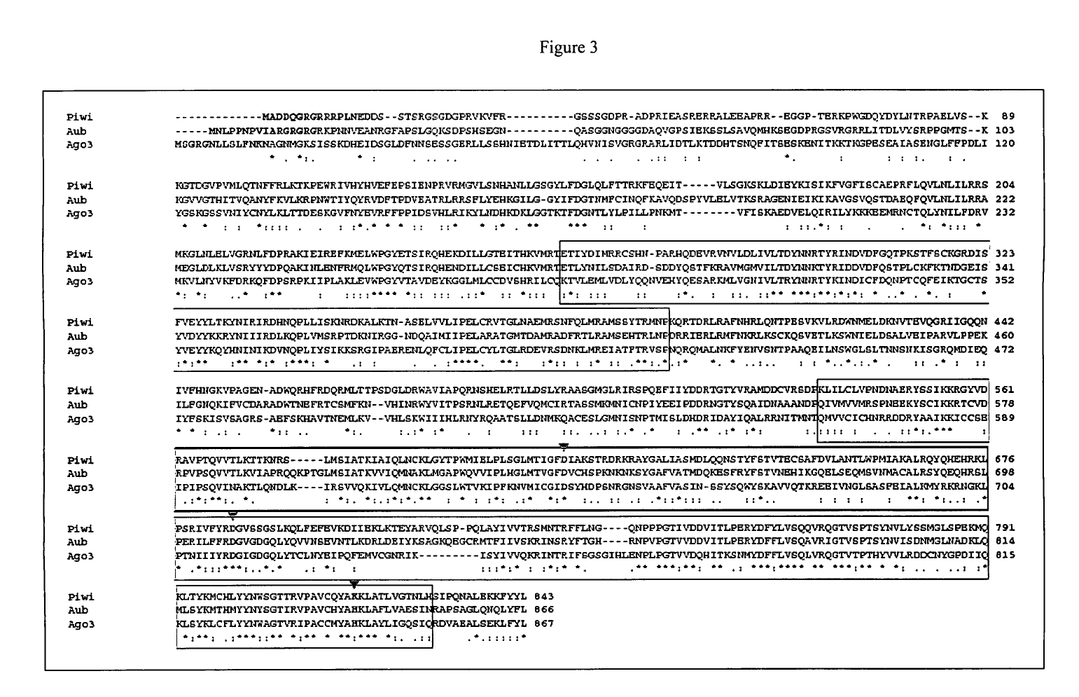

In Drosophila, the Piwi-clade of Argonaute proteins consists of the three family members Piwi, Aubergine (Aub) and Ago3. In contrast to the euchromatic and well studied aub and piwi genes, the predicted ago3 gene (CG40300) resides in the pericentromeric heterochromatin of chromosome 3L (cytological position 80F). Although germline enriched expression of ago3 has been demonstrated by in situ hybridization (Williams and Rubin, 2002), an experimentally determined sequence of the Ago3 protein has not been reported. As a prelude to further studies of this family member, we sequenced several available ago3 cDNAs, the longest of which (RE57814) corresponds to a 2.7 kb cDNA originating from a 133 kb locus. This contains a presumably complete open reading frame of 867 amino acids, which encodes the PAZ and PIWI domains that are a signature of this family (FIG. 3).

Armed with the complete coding sequence of all three family members, we raised polyclonal antibodies that recognize the amino-terminal 15 residues of Piwi, Aub and Ago3, a region that is highly diverged among these proteins (FIG. 3). Western blot was performed on total protein lysates from female carcasses (flies with ovaries removed), ovaries and 0-2 hr embryos using antibodies raised against Piwi, Ago3 and Aub. Western blotting indicates that each antibody recognizes an approximately 85 kDa protein from ovary extract which is not detectable in extracts from female carcasses. The Piwi and Ago3 antibodies recognize additional bands, none of which was enriched in upon immunoprecipitation. All three proteins are detectable in extracts from 0-2 hr embryos, suggesting that each is maternally deposited into the developing egg.

The specificity of each antibody for its intended target was verified by mass spectrometric analysis of immunoprecipitates from ovary extracts. Western blot analysis was performed on immunoprecipitations prepared with Piwi, Ago3 and Aub specific antibodies from ovary extract. Immunoprecipitates, as well as the total extract and supernatant from the immunoprecipitate were blotted individually with each of the three Piwi family antibodies. In each case, the target protein was recovered without immunoprecipitation of other family members. Specificity was also demonstrated by examining immunoprecipitates of each Piwi family member by Western blotting. Again, each antibody specifically immunoprecipitated its respective target without recovery of its related siblings.

Previous studies have used myc-tagged Piwi and GFP-tagged Aub transgenes to investigate the spatial and temporal expression patterns of these proteins during oogenesis (Cox et al., 2000; Harris and Macdonald, 2001). We used our specific Piwi family antibodies to examine expression patterns of the endogenous proteins and to extend analyses to the third family member, Ago3.

First of all, cell type-specific and subcellular localization of endogenous Piwi family members in developing ovarioles were examined. An overview of Piwi localization in the ovariole, and a detailed view of the germarium containing the two stem cells were obtained. The overlap between Piwi and DNA staining indicates enrichment of Piwi in the nuclei of all cells. Nuclear localization of Piwi was apparent in nurse cell and surrounding somatic follicle cells. A weak accumulation of maternally deposited Piwi protein at the posterior pole of stage 10 oocytes was also observed. Similarly, an overview of Aubergine localization in the ovariole was obtained. We found an enrichment of Aub in at the posterior pole of the developing oocyte, an Aub localization in the germarium with the germline stem cells, and enrichment of Aub in the cytoplasm and the perinuclear nuage in the germline. Staining is absent, however, from the surrounding somatic follicle cells. We also found substantial accumulation of Aub at the posterior of a stage 10 oocyte. Similarly, examination of an overview of Ago3 localization in the ovariole and a detailed view of Ago3 staining in the germarium shows strong enrichment around the stem cell nuclei and in discrete foci. We also found an Ago3 localization to nuage in nurse cells.

Thus, immunofluorescence and confocal microscopy revealed that all three proteins are present in the germline lineage beginning in the stem cell and extending through the mature oocyte. However, each protein showed characteristic patterns of subcellular and tissue localization. As previously reported (Cox et al., 2000), Piwi is a predominantly nuclear protein that is present not only in germline cells but also in the somatic cells of the ovary. For example, strong Piwi staining is seen in the cap cells that surround the germline stem cells and in the follicle cells that envelop the developing egg chamber. In later stage egg chambers, Piwi is detectable in the cytoplasm of the developing oocyte with a slight enrichment at the posterior where germline primordia of the embryo will form. An examination of early embryos confirmed the accumulation of maternally deposited Piwi protein in pole plasm.

In contrast to Piwi, Aubergine is expressed at very low or undetectable levels outside the germline cell lineage. Furthermore, Aub is primarily cytoplasmic. As reported previously for GFP-Aub, we detect endogenous protein in the germline stem cells, the developing cystoblasts and the nurse cells of developing egg chambers. Aubergine is enriched in nuage, a perinuclear, electron dense structure, displaying a localization pattern very similar to the nuage marker, Vasa. As is observed for Vasa, Aubergine is deposited into the developing oocyte from early stage 10 onwards and becomes localized to the pole plasm.

As with Aubergine, Ago3 expression is predominantly cytoplasmic. It is present in the germline lineage but is not detectable in the somatic cells surrounding the egg chamber, although we do find Ago3 in the somatic cap cells of the germarium. Ago3 shows a more striking accumulation in nuage than does Aub, and it is also found in prominent but discrete foci of unknown character in the germarium. Despite its localization in nuage, Ago3 is unlike Vasa and Aub in that it does not accumulate at the posterior pole of the developing oocyte, and Ago3 is not detected in pole plasm in early embryos. In many ways, the Ago3 expression pattern resembles that of another nuage component, Maelstrom, a conserved protein of unknown function that is required for germline development (Findley et al., 2003).

Considered together, our results indicate that all three Drosophila Piwi proteins show specialized patterns of cell type-specific expression and subcellular localization in the ovary. This is consistent with genetic studies showing that Piwi and Aub have non-redundant but essential functions in oogenesis and predicts that disruption of Ago3 might also impact fertility irrespective of Piwi and Aub status.

To investigate the small RNA populations bound by the three Drosophila Piwi family members; we immunoaffinity purified each RNP complex from ovary lysates. Radioactively labeled RNA isolated from specific Piwi family RNPs were analyzed on a denaturing polyacrylamide gel. The results indicated that all three proteins associate with small RNAs ranging in length from 23 to 29 nt. 2S rRNA was also shown to be present in purifications.

By comparison, labeling of small RNAs isolated from Agol RNP complexes that are known to contain miRNAs revealed a discretely sized population of around 22 nt (21-23 nt) long microRNAs under identical conditions.

To explore the sequence content of Piwi-bound small RNAs, we prepared cDNA libraries from RNAs recovered from Piwi, Aub and Ago3 complexes. In parallel we prepared a cDNA library from 23-29 nt RNAs purified from total ovary RNA. Large-scale sequencing of these libraries yielded a total of 60,691 reads (17,709 for Piwi, 23,376 for Ago3, 14,872 for Aub and 4734 for ovary total RNA, respectively) that match perfectly to Release 5 of the Drosophila melanogaster genome or to non-assembled Drosophila sequences from Genbank. These were used for subsequent analysis.

The first indication that the three Piwi proteins bound different small RNA populations came from the size distribution of the sequences obtained from each complex (FIG. 1). With an average length of 25.7 nt, Piwi-associated RNAs are significantly longer than Aub-associated (24.7 nt) or Ago3-associated (24.1 nt) RNAs. This subtle difference is also apparent from the mobility of these RNA populations on denaturing polyacrylamide gels.

Additional differences emerge from an analysis of the nucleotide bias of the 5′ ends of the RNAs. While Piwi and Aub bound RNAs have a strong preference for a terminal uridine (83% and 72%, respectively) and thus resemble microRNAs and mammalian piRNAs, this trend is essentially absent in the Ago3 bound population (37% terminal U).

An analysis of the sequences derived from each Piwi complex indicated that the Piwi family-bound small RNA populations are quite complex. Most of the small RNAs in each case were cloned only once (87% for Piwi, 81% for Aub and 73% for Ago3). Additionally, only 1.5% of sequences in all three libraries combined were cloned more than 10 times. Considered together, these data suggest that our characterization of Piwi-bound RNAs is far from saturating. Moreover, we detected no common sequence motifs either within the RNA sequences themselves or by examination of their sequence contexts in the genome.

Despite their differences, the small RNA populations obtained from each complex were remarkably similar in the types of genomic elements to which they correspond. All sequences were categorized using public databases and additional annotation of the Release 5 assembly of the Drosophila melanogaster genome (see Materials and Methods). Overall, more than three quarters of all sequences from each of the three complexes could be assigned to annotated transposons or transposon remnants, with nearly all identified transposons and transposon classes (non-LTR and LTR retrotransposons and DNA transposons) being represented. An additional 1 to 5% of small RNAs were derived from regions of local repeat structure, such as the subtelomeric TAS repeats or pericentromeric satellite repeats. Thus, nearly 80% of Piwi bound RNAs in Drosophila can be characterized as rasiRNAs. Less than 10% of the RNAs derived from each complex (5.5% for Piwi, 9.4% for Aub and 5.3% for Ago3) map to annotated abundant non-coding RNAs including rRNAs, tRNAs, snoRNAs. As these are derived almost exclusively from the sense strand, they could arise from a contamination of our preparations with nonspecific degradation products. Less than 5% (4.2% for Piwi, 4.3% for Aub and 1.0% for Ago3) of Piwi-interacting RNAs map to exons or introns of annotated protein coding genes with around 90% of these originating from the sense strand. Only a small number of microRNA sequences were obtained (0.3% for Piwi, 0.4% for Aub and 1.8% for Ago3), confirming the previously reported separation of the rasiRNA and miRNA pathways. The remaining sequences (10.2% for Piwi, 6.4% for Aub and 4.6% for Ago3) map to completely unannotated regions of the genome. Interestingly, these regions correspond to heterochromatic, transposon-rich loci.

Thus, Drosophila Piwi-interacting RNAs share both similarities and differences with mammalian piRNAs. In both flies and mammals, Piwi-associated RNAs are significantly longer than microRNAs and are found specifically in reproductive tissues. Also, Piwi-interacting RNAs from both species are very complex populations that appear to have no unifying sequence motif. At least Piwi- and Aub-bound populations show a preference for a 5′U residue, as do mammalian piRNAs. However, unlike mammalian piRNAs, which are relatively depleted of sequences that correspond to transposons and repeats, the vast majority of Drosophila piRNAs match to repetitive elements and can be classified as rasiRNAs. In fact, only about 20-25% of Drosophila piRNAs can be mapped to unique locations in the genome as compared to more than 85% of mammalian piRNAs. We therefore propose to classify Drosophila rasiRNAs as a subset of the broader class that has been termed piRNAs.

### Example II

### Drosophila piRNAs are Derived from Discrete Genomic Loci

The small RNA sequence data obtained from the three Piwi complexes is consistent with previous reports that have proposed a role for these proteins in transposon regulation (Saito et al., 2006; Vagin et al., 2006). We wished to exploit the depth of our sequence analysis to investigate how the small RNA-based transposon control program is established. Potentially, transcripts from every transposon could serve as templates for the production of small RNAs. This is the likely model through which plants silence transposons, via a mechanism that depends upon RNA-dependent RNA polymerases to generate dsRNA silencing triggers. Alternatively specialized transposon control regions could produce piRNAs whose complementarity with transposons allows efficient silencing of dispersed elements in trans. It was therefore essential to understand the genomic origin of the Drosophila piRNAs.

In Drosophila, intact and potentially active transposable elements populate the euchromatic chromosome arms as well as pericentromeric and telomeric heterochromatin. There are also numerous transposon remnants that, although generally recognizable, have been mutated to such a degree that they are unlikely to conserve even the potential for transposition. These are strongly enriched in the beta-heterochromatin that is found bordering Drosophila centromeres and are generally absent from euchromatic chromosome arms (Hoskins et al., 2002). Given that small RNAs associated with each of Piwi proteins correspond to vast majority of all known transposons, it is not surprising that a depiction of the chromosomal locations matched by these RNAs closely resembles a plot of transposon density. However, since each transposon is generally present at multiple chromosomal locales, such a plot can not provide unambiguous information about genomic origin of piRNAs.

To address the genomic origin of piRNAs it was necessary to restrict our analysis to the 20-25% of piRNAs that match the genome at a unique position, allowing an unambiguous assignment of their point of origin. A density plot of this small RNA subset shows a striking clustering of piRNAs at discrete genomic loci. A similar plot can be obtained for those RNAs that match the genome in multiple locations if we simply weight the signal from each piRNA-genomic match as the reciprocal of its genomic frequency. These data indicated that at least a subset of Drosophila piRNAs are derived from discrete genomic loci, similar to those that have recently been reported for mammalian piRNAs.

We next produced a catalog of the loci that generate piRNAs in the Drosophila ovary. For each locus to be tagged confidently as a source of piRNAs, we required that it produce both numerous piRNAs and piRNAs that mapped uniquely to that site (see Methods). In this way, we identified 134 genomic locations that can be identified with high confidence as sites of piRNA generation. These clusters accommodate 81% of all piRNAs that match the genome at a single site. Although these sites comprise only 5% of the assembled genome (6.8 MB out of 137 MB), more than 92% of the sequenced piRNA population could potentially be derived from these loci.

Only 8% of the clusters are found in euchromatic regions, with the remainder being present in pericentromeric and telomeric heterochromatin. Telomeric clusters are most often composed of satellite sequences and correspond to the subtelomeric Terminal Associated Sequence (TAS) repeats. These separate the euchromatic chromosome arms from the tandem repeats of HetA and TART transposons, which comprise the Drosophila telomeres (Karpen and Spradling, 1992). Although subtelomeric TAS repeats and especially telomeric HetA and TART transposon repeats are not complete in the current genome assembly, we do find sequences corresponding to both components of Drosophila telomeres. Therefore, TAS repeats and HetA and TART retrotransposons can be considered as part of combined telomere-terminal clusters. The presence of uniquely mapped piRNAs allows us to conclude that most telomeres (X, 2R,2L, 3R) harbor piRNA clusters. Interestingly, both components of telomeric clusters preferentially correspond to piRNAs found in Ago3 and Aub complexes. Clusters found in the pericentromeric beta-heterochromatin display a high content of sequences matching annotated transposable elements (typically from 70 to 90%) with the majority being partial or defective copies. Transposons within these clusters may be inserted within each other or arranged in tandem. Generally, these pericentromic clusters generate piRNAs that join all three complexes.

The size of Drosophila piRNA clusters varies substantially with the smallest being only a few kB and the largest being a 240 kB locus in the pericentromeric heterochomatin of chromosome 2R (cytological position 42AB). This largest cluster accommodates 20.8% of all uniquely mapping piRNA sequences and could potentially give rise to 30.1% of all the piRNAs, which we identified (Table 1). Even taking into account its large size, this represents an ˜150-fold enrichment for sites that match to sequenced piRNAs in comparison to the annotated genome. Overall, the largest 15 clusters (Table I) account for 50% of the uniquely mapping and potentially accommodate 70% of the total piRNA population.

We also showed that flamenco is a piRNA cluster. The most proximal 1.2 Mb of pericentromeric heterochromatin on the X chromosome was studied. The positions of three large piRNA clusters (numbers correspond to table 1) were identified, and mapped to the position in the Drosophila Genome Assembly, Release 5 in nt. The density of uniquely mapping piRNAs was determined. Cluster #8 corresponds to the flamenco locus. A more detailed map showing on the flamenco cluster also include protein coding genes that flank the cluster. In addition, a map of annotated transposons indicated LTR elements and LINE elements was mapped to the same. The flamenco cluster ends 185 kb proximal to DIP1 in a gap of unknown size. Many retroelements, Gypsy, Idefix and ZAM were known to be regulated by the locus. The first 20 kb of the flamenco locus displaying the flanking DIP1 gene, annotated transposon fragments, the P-element insertion that results in an inactive flamenco allele, and the density of all Piwi associated piRNAs that potentially map to this region were also identified. We note that over 99% of the uniquely mapping piRNAs are derived from one (the top) strand.

In mammals, piRNA clusters show profound strand asymmetry. However, in flies, even uniquely mapping piRNAs most often arise from both strands of a cluster. While this might be interpreted as suggestive of a dsRNA precursor to mature piRNAs, there are clusters that show marked strand asymmetry. For example, two clusters at cytological position 20A on the X chromosome produce uniquely mapping piRNAs only from one strand. This suggests that, as was proposed for mammals, piRNAs in D. melanogaster could be derived from single-stranded RNA precursors.

Our results suggest that a limited number of predominantly heterchromatic loci can produce the majority of Drosophila piRNAs. These share superficial similarities with mammalian piRNA clusters. However, there are also notable and important differences. Chief among these are the production of small RNAs from both strands and a striking enrichment for transposon sequences, which strongly implicates Piwi complexes in transposon control in Drosophila germline.

### Example III

### piRNA Clusters are Master Regulators of Transposon Activity

Numerous genetic studies have pointed to discrete genomic loci that suppress the activity of specific transposons. The best understood of these is the recessive flamenco/COM locus that comprises a large region at the distal end of the pericentromeric beta-heterochromatin of the X-chromosome (Prud'homme et al., 1995). The flamenco locus was originally identified because it controls the activity of the retroviral gypsy element (Pelisson et al., 1994). This locus has subsequently been shown to suppress two additional retroelements, Idefix and ZAM (Desset et al., 2003). In flamenco mutant females, the normally tight control over these three elements is lost, resulting in high transposition rates. Through the use of numerous deficiencies, flamenco has been mapped proximally to the Dip-1 gene and is proposed to span a region of at least 130 kB. Since rescue experiments have indicated that flamenco is not Dip-1 (Robert et al., 2001), no protein coding candidate corresponding to flamenco presently exists.

Our data strongly suggest that the genetically mapped flamenco function corresponds to a piRNA cluster (cluster #8, Table I). The genomic sequence proximal to DIP1 contains numerous nested transposable elements spanning a total length of 185 kb, where a gap of unknown size in the Release 5 genome assembly separates the flamenco locus from more proximal heterochromatic sequences. This locus contains numerous fragments of all three transposable elements that have been shown to be de-repressed in flamenco mutants (gypsy, Idefix and ZAM) in addition to many other families of transposons.

The piRNA cluster at the flamenco locus gives rise to 2.2% of uniquely mapping piRNAs and potentially accommodates 13.3% of all piRNAs, thus representing one of the biggest piRNA clusters in the Drosophila genome. Nevertheless, the cluster is enriched for piRNAs targeting transposons that are controlled by flamenco; 79% of all piRNAs that target ZAM, 30% of those matching Idefix and 33% of RNAs complementary to gypsy can be attributed to this single locus.

Considering sequences that map uniquely to genome, this cluster is one of only two, which produce piRNAs with a marked strand asymmetry. The vast majority of transposons are similarly oriented within the flamenco region. Thus, both strand asymmetry and the observed enrichment for piRNAs that are antisense to transposons can be achieved by generating piRNAs from a long, unidirectional transcript that encompasses the locus. Such a model is consistent with the observation that we identify many piRNAs from this cluster, and the others, which cross the boundaries of adjacent transposons. The only molecularly defined flamenco mutation corresponds to a P-element insertion ˜2 kb proximal to DIP1 (Robert et al., 2001). The insertion point is located 550 bp upstream of first piRNA uniquely mapped to this cluster. Considering these observations as a whole leads to a model wherein the P-element insertion inactivates flamenco by interfering with the synthesis of the piRNA precursor transcript.

Additional support for the model comes from the observation that flamenco-mediated silencing of gypsy depends on piwi. Notably, the piRNA cluster at the flamenco locus preferentially loads the Piwi protein, with 94% of its uniquely mapping RNAs being Piwi partners. This preferential loading is nearly unique among the clusters that we have identified. Moreover, all three of flamenco-regulated retroelements are preferentially or exclusively transcribed in somatic follicle cells, where Piwi itself is the predominant family member. Thus, our data strongly suggest that flamenco corresponds to a piRNA cluster that is preferentially expressed in follicle cells where it programs Piwi complexes for transposon silencing.

The second piRNA cluster that has been genetically linked to transposon control corresponds to the subtelomeric TAS repeat on the X-chromosome (Table I, cluster #4). This cluster differs from pericentromeric piRNA loci in that it consists of mainly locally repetitive satellite sequences. Numerous studies indicate that insertions of one or two P-elements into X-TAS are sufficient to suppress P-M hybrid dysgenesis (Marin et al., 2000; Ronsseray et al., 1991; Stuart et al., 2002). Transposon silencing by these insertions has been linked to the Piwi family, as it is relieved by mutations in aubergine (Reiss et al., 2004). The precise insertion sites of three suppressive P-elements in X-TAS have been mapped and they correspond to areas of this locus, which give rise to multiple small RNA sequences bound by all three Piwi family proteins with preference for Ago3 and Aub. These data clearly suggest that X-TAS acts as a master control locus that can be programmed by transposon insertion to regulate the activity of similar elements in trans. In accord with a trans-acting model for suppression, defective, lacZ-containing P-elements inserted into X-TAS can suppress euchromatic lacZ transgenes in the female germline (Roche and R10, 1998; Ronsseray et al., 1998).

The combination of existing genetic data with our mapping of piRNA clusters strongly supports a model in which these serve as master control loci for transposon suppression. This clearly contradicts a purely copy number-based model for transposon control and raises the question of whether dispersed transposon copies play any role other than that of silencing targets.

### Example IV

### Argonaute 3 Show a Preference for Sense Strand piRNAs

Recent studies have indicated that Drosophila rasiRNAs show a strong bias for sequences that are antisense to transposable elements, as would be expected for suppressors of transposon activity. We asked whether this observation held for our sequenced piRNAs by examining the strand bias profiles of those that appeared in Piwi, Aub and Ago3 complexes. We aligned our piRNA sequences to a comprehensive database of consensus sequences for D. melanogaster transposable elements (transposon sequence canonical sets v9.41, Flybase). Since the actual transposon sequences in the genome can significantly diverge, we performed this analysis at several stringency levels, allow from zero to 5 mismatches to the consensus. Overall, we uncovered pronounced strand asymmetry in each complex. Piwi and Aub preferentially incorporate piRNAs matching the antisense strand of transposable elements. In contrast, Ago3 complexes contain piRNAs that are strongly biased for the sense strand of transposons. In total, 76% of the piRNAs associated with Piwi and 83% of those in Aub RNP complexes corresponded to transposon antisense strands; whereas 75% of the Ago3 bound piRNAs correspond to transposon sense strands.

The pattern of asymmetry among the three RNPs is preserved when we evaluated each transposable element separately. This was true irrespective of the transposon class with LINE elements, retroelements and inverted repeat (IR) elements behaving identically. As an example, a plot of piRNAs along the consensus sequence of the F element reveals numerous antisense piRNAs that are loaded into Piwi and Aub and numerous sense piRNAs that enter Ago3 complexes (result not shown). There are a very few notable exceptions where asymmetry remains marked but is reversed for Piwi/Aub and Ago3 complexes (for example, accord2, gypsyl2, diver2 and hopper2). Interestingly, the frequency of piRNAs corresponding to each transposon varies widely depending upon the identity of the element. Roo, R1A1 and the F and Max elements are among the most highly represented. It is presently unclear whether differences in abundance reflect differences in the activity of transposons in our strain.

To assess the relative abundance of piRNA populations bound to each of the three Piwi proteins in the ovary we compared profiles for each individual RNP complex to the profile obtained from piRNAs cloned from total ovary RNA. The pattern that emerged from the total piRNA population closely resembled that of the Piwi and Aub complexes. This indicates that sense-oriented piRNAs in Ago3 complexes are less abundant overall.

Our analyses of the flamenco cluster were consistent with a model in which single stranded precursors from piRNA loci give rise to predominantly antisense piRNAs. The discovery of sense strand piRNAs in Ago3 complexes instead raised the possibility of double-stranded precursors to piRNAs. To begin to distinguish between these models, we examined the strand bias of each of the three Piwi complexes at several piRNA loci. As an example, the largest piRNA cluster in the Drosophila genome, at 42AB, contains a high density of transposon sequences, as was observed for flamenco. Most are degenerated transposon copies unlikely to be capable of mobilization. Unlike flamenco, transposons within 42AB are oriented in either direction, without an apparent bias. The 42AB cluster produces uniquely mapping piRNAs from both strands. Interestingly, just as is observed in an analysis of transposon consensus sequences, strand asymmetry is preserved in these uniquely mapped RNAs within this single locus. An interesting example is two tandem BATUMI elements that exist in opposite orientations. Uniquely mapping RNAs in the Ago3 complex correspond to the sense strand of both copies. Overall, the pattern of Ago3-bound piRNAs presents almost a mirror image of the pattern of Piwi and Aub-associated RNAs.

Overall, these results show that individual Piwi complexes show profound strand biases. Applicants have generated a heat map indicating the strand bias of cloned piRNAs with respect to canonical transposon sequences (not shown). In that map, transposons are grouped into LTR elements, LINE elements and Inverted Repeat elements and sorted alphabetically. The ratio of sense to antisense sequences were determined. The cloning frequency for individual transposons in all three complexes was indicated as a heat map. Applicants also determined the density of all cloned piRNAs assigned to the canonical F-element sequence (not shown). Three mismatches were allowed for this mapping. Frequencies in each Piwi family RNP are shown individually in the map. A graph of piRNA matches in the total ovary sample was prepared. In addition, Applicants also determined the density of Ago3 piRNAs as compared to the density of RNAs found in Piwi and Aub (not shown). The map is shown for uniquely mapping piRNAs only in the largest genomic cluster at cytological position 42AB. Annotated transposon fragments were included.

### Example V

### A Relay between piRNA Clusters and Dispersed Transposable Elements

The detection of small RNAs from both strands of transposons and the involvement of Argonaute family proteins hints at a double-stranded RNA precursor to piRNAs. However, given our current understanding of how dsRNAs are processed by RNAse III enzymes and loaded into Argonaute proteins, it is difficult to understand how individual Piwi complexes could accurately distinguish between sense and antisense strands of transposons. Transposon-related sequences that give rise to piRNAs lack a significant bias in their orientation within most loci. If long transcripts traversing piRNA loci act as precursors, transposon strand information should be largely absent from the piRNA clusters. Dispersed and active transposon copies produce predominantly or exclusively sense transposon transcripts. We therefore hypothesized that transcripts from dispersed copies might contribute strand specificity during piRNA biogenesis, perhaps interacting with transcripts from piRNA loci to produce double stranded RNAs that are processed by a Dicer-like mechanism.

To address this possibility, we examined the relationship between the sense and antisense piRNAs corresponding to each element. A biogenesis mechanism resembling siRNAs or miRNAs would predict the detection of sense-antisense piRNA pairs that reflect the 2 nucleotide 3′ overhangs produced by RNAse III enzymes. According to this scenario, complementary sense and antisense piRNAs should have 5′ ends separated by 23 nucleotides (2 nucleotides less than the average piRNA size of 25 nucleotides) and correspondingly show 23 nucleotides of complementary sequence. To probe this possibility, we searched for common patterns in the distance separating the 5′ ends of piRNAs from each genomic strand. Applicants first generated a frequency map of the separation of piRNAs mapping to opposite genomic strands. The spike at position 9 (the graph starts at 0) indicates the position of maximal probability of finding the 5′ end of a complementary piRNA. In other words, plotting the frequency of each observed degree of separation, we failed to see the expected peak at 23 nucleotides. Instead, we found that 5′ ends of complementary piRNAs tend to be separated by only 10 nucleotides.

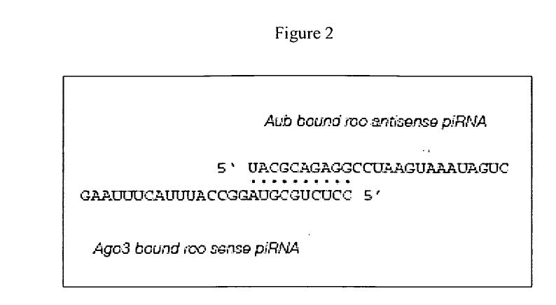

To probe the significance of this observation, we performed an additional test. We extracted the first 10 nucleotides of each piRNA. This sequence was then compared to the piRNA database to identify complementary sequences (e.g., measuring the frequency with which a perfectly complementary 10-mer could be found at each position within the piRNAs in the complete database). The positions of the complementary 10-mers within their host piRNAs were tallied are presented graphically. Similar analyses in which each 10 mer beginning in positions 2-10 failed to yield enrichment for complementary sequences at any position within the piRNA population. For purposes of presentation, results from each position, other than position 1, were averaged and presented with error bars showing the standard deviation from the mean. The result shows that 20% of all terminal 10-mers have a complementary sequence that begins at position 1 of another piRNA. No enrichment is seen for complementary 10-mers beginning at any other position. An example of one sense-antisense piRNA pair targeting the roo transposon is shown in FIG. 2. This is an individual example of two cloned piRNAs which overlap with the characteristic 10 nt offset, with the 5′U of the Aub bound roo antisense piRNA, and the A at position 10 of the Ago3 bound roo sense piRNA.

The observed 10 nt offset between antisense pairs of piRNAs failed to support a conventional model in which dsRNAs are processed by RNAseIII family enzymes to produce sense and antisense piRNAs. Instead, the 10 nucleotide overlap between these RNAs provoked the hypothesis that the Piwi proteins themselves might have a role in piRNA biogenesis. According to such a model, a Piwi-piRNA complex would recognize and cleave a transposon transcript. This cleavage event would occur, by extension from other Argonaute proteins, at the phosphodiester bond across from nucleotides 10 and 11 of the piRNA, generating a 5′ monophosphorylated end 10 nucleotides distant, and on the opposite strand, from the end of the original piRNA. The cleaved product would be loaded into a second Piwi family protein, ultimately becoming new piRNA after processing at the 3′ end by an unknown mechanism. This would produce the observed 10 nt offset between 5′ ends of sense and antisense sequences. Although the biochemical activities of the Piwi family proteins have not been extensively studied, both Drosophila Piwi (Saito et al., 2006) and Rat Riwi (Lau et al., 2006) proteins have been demonstrated to cleave targets in a small RNA-guided fashion. Moreover, both Aubergine and Ago3 contain the DDH residues that form the active site of the RNAse H-like motif within the Piwi domain (See FIG. 3).

The predominance of sense transposon sequences in the Ago3 complex suggests that this family member incorporates piRNAs following cleavage of transcripts as directed by antisense piRNAs that populate Piwi and/or Aub complexes. This is consistent with the lack of a strong U-bias at the 5′ end of Ago3-bound piRNAs. However, a strong prediction of such a biogenesis model is that the 10th position of Ago3-bound RNAs would correspond to a site that is complementary to the first position of antisense piRNAs (see FIG. 2). Since Piwi and Aub-bound small RNAs have strong preference for a U at the 5′ position, position 10 of Ago3-bound piRNAs should be enriched for A. A nucleotide bias plot for all three family members matches this prediction with 73% of all Ago3 piRNAs having an A at position 10. Interestingly, this trend is observed not only for small RNAs that have 10 nt offset partner (84%), but also for sequences that do not have partner in our dataset (63%) suggesting that vast majority of Ago3-associated piRNAs may be produced by the Piwi-mediated cleavage mechanism.

Ago3 piRNAs could potentially be generated following cleavage of a target by antisense piRNAs loaded into either Piwi or Aub complexes. This led us to explore in more detail the relationship between the sense and antisense piRNAs in each of the three complexes.

We quantified the frequency with which complementary RNAs, with a 10 nucleotide offset at their 5′ ends, appeared in pair wise comparisons of each library. Heat maps that indicated the degree to which complementary 5′ 10-mers are found in pair wise library comparisons, with different intensity of the signal were generated. Redundant sequences within each library were eliminated. A control analysis was performed with the 10-mer from position 2-11. The strongest relationship was detected between Ago3 and Aub-associated RNAs. Even though our sequencing efforts are unlikely to be saturating, more than 48% of small RNAs in the Ago3 library had complementary partners in the Aubergine-bound small RNA collection. If cloning frequencies are eliminated to create non-redundant collections of piRNAs, more than 30% of Ago3-bound RNAs have complementary partners in Aubergine. Statistically significant, although less pronounced, interactions are indicated between Piwi and Ago3. No significant enrichment for complementary piRNA pairs is seen between Piwi and Aub. Interestingly a self-self comparison of Ago3 complexes does show enrichment for complementary sequences. Thus, our data suggest that Ago3-associated sequences may be produced by Aub-guided cleavage with contribution from Piwi complexes and Ago3 complexes themselves.

Considered together, the aforementioned analysis strongly suggests that Aub-mediated cleavage of transposon transcripts creates the 5′ ends of new piRNAs that appear in Ago3. If the reciprocal process also occurred, then sense and antisense piRNAs could participate in a feed-forward loop to increase production of silencing-competent RNAs in response to the expression of specific repetitive elements. Since Argonautes act catalytically, a significant amplification of the response could be achieved by even a relatively low level of sense piRNAs in Ago3 complexes. This model predicts that piRNAs participating in this process, namely those with complementary partners, should be more abundant that piRNAs without detectable partners.

To test this hypothesis, we sorted piRNA sequences by their abundance as reflected by their cloning frequency. Specifically, ten bins were constructed for each Piwi complex and for all sequences combined by dividing sequences according to their cloning frequency. For example, the bin labeled 0-10 contains the 10% of sequences that were most frequently cloned. The fraction of sequences within each bin that has a complementary partner was then graphed on the Y-axis. Indeed, the most frequently cloned Aub and Ago3-associated piRNAs show an increased probability of having antisense partners within the dataset. Interestingly, Piwi-associated RNAs do not follow this pattern.

### Example VI

### A Model for Transposon Silencing in Drosophila

Our data point to a comprehensive strategy for transposon repression in Drosophila that incorporates both a long-term genetic memory and an acute response to the presence of potentially active elements in the genome. We propose that the piRNA loci themselves act as an initial source for piRNAs that provide a basal resistance to the sum of transposable elements with which Drosophila melanogaster has adapted to co-exist.

Presently, the biogenesis pathway for primary piRNAs remains obscure. Several lines of evidence suggest that the piRNA precursor is a long, single-stranded transcript that is processed, preferentially at U residues, to yield 5′ monophosphorylated piRNA ends. We detect transcripts from piRNA loci by RT-PCR that cross the boundaries of several of their constituent transposable elements (not shown). We also find numerous small RNAs that cross junctions between two individual transposons, as would be expected if piRNA loci encode contiguous precursor transcripts. Finally, the existence of loci like flamenco that produce piRNAs from only one genomic strand indicates that piRNAs may be processed from single-stranded precursors. Based upon these observations, it is likely that formation of primary piRNAs in both Drosophila and mammals occurs through a similar mechanism.

The generation of piRNA 3′ ends occurs via an equally mysterious process. Mature piRNAs could be generated by two cleavage events and subsequently loaded into the appropriate Piwi complex. Alternatively, the 3′ ends of piRNAs could be created following 5′ end formation and incorporation of a long RNA into Piwi by either endo- or exo-nucleolytic resection of 3′ their ends. The latter model is attractive since it could provide an explanation for observed size differences between RNAs bound to individual Piwi proteins, a feature common to both D. melanogaster and mammalian piRNAs. For example, characteristic sizes could simply reflect the footprint of individual Piwi proteins protecting their bound RNAs from the 3′ end formation activity. The reported modification of the 3′ ends of piRNAs (Vagin et al., 2006) could occur after processing in either model.

Primary piRNAs could be incorporated into Piwi or Aubergine complexes or both. Given observations from the flamenco locus, it is almost certain that Piwi is able to incorporate primary piRNAs. In accord with this model, Piwi-associated sequences demonstrate greater diversity than piRNAs bound to Aub and Ago3, whose bound populations might be skewed by their participation in an amplification loop.

Once primed with a primary piRNA, Piwi-family complexes use these as guides to detect and cleave transcripts arising from potentially active transposons. This cleavage event, opposite nucleotides 10-11 of the piRNA, can generate the 5′ end of a new sense-oriented piRNA that is derived directly from transposon mRNA and is most often incorporated into Ago3. Again, the mechanism that generates the 3′ end of these secondary small RNAs remains obscure. We have yet to determine whether Ago3 bound piRNAs are modified at their 3′ ends as are those in Aub and Piwi complexes (Vagin et al., 2006).

Once loaded with sense piRNAs, the Ago3 complexes seek out antisense transcripts and direct their cleavage. We imagine that the principal source of antisense transposon sequences are transcripts derived from the piRNA clusters. Thus, clusters not only represent the source of primary piRNAs but also participate in production of secondary piRNAs working as relay stations in an amplification loop. While the primary piRNA biogenesis mechanisms may sample the cluster at random, cleavage of cluster-derived transcripts by Ago3 would skew the production of secondary piRNAs to those that are antisense to actively expressed transposons. This would not only increase the abundance of those RNAs needed to combat potentially mobile elements but also explain the enrichment of antisense sequences within Aub, even from clusters without a pronounced orientation bias in their constituent transposons. Multiple turnover cleavage by Ago3 would magnify the potential of the feed-forward loop to reinforce the silencing response. Individual clusters may interact with each other, just as they can interact with dispersed transposon copies, to amplify silencing potential. This is supported by the observation that Ago3-associated piRNAs that are unambiguously derived from the clusters still show a strong preference for A at position 10.

All three Piwi proteins are loaded maternally into the developing oocyte (Harris and Macdonald, 2001; Megosh et al., 2006). At a minimum, both Piwi and Aub are concentrated in the pole plasm, which will give rise to the germline of the next generation. Coincident deposition of bound piRNAs could provide enhanced resistance to transposons that are an ongoing challenge to the organism, augmenting any low level of resistance that may be provided by zygotic production of primary piRNAs. Indeed, maternally loaded rasiRNAs were detected in early embryos (Aravin et al., 2003) and their presence was correlated with suppression of hybrid dysgenesis in D. virilis (Blumenstiel and Hartl, 2005). Maternal deposition of silencing complexes and the existence of an amplification loop may also explain one of the most curious aspects of hybrid dysgenesis. Establishment of transposable element silencing often shows genetic anticipation, requiring multiple generations for a repressive locus to achieve its full effect. According to our model, a single generation may not be enough for full operation of a feed-forward loop to create an effective silencing response to some transposons, particularly if sequences that correspond to those elements within piRNA clusters are particularly diverged or present at low copy number.

In C. elegans, effective silencing by RNAi depends upon an amplification mechanism that triggers production of secondary siRNAs (Sijen et al., 2001). The primary dsRNA trigger cannot provide an effective silencing response and seems largely dedicated to promoting the use of complementary targets as templates for RNA-dependent RNA polymerases (RdRPs) in the generation of secondary siRNAs. This mechanism produces a marked asymmetry in the secondary siRNA population similar to that which we observe in piRNAs in the ovary total RNA sample. Similar secondary siRNA production cycles are also likely to be key to effective silencing in plants and to maintenance of centromeric heterchromatin in S. pombe, processes which both depend upon RdRP enzymes (reviewed in Herr, 2005; Martienssen et al., 2005).

In Drosophila, no RdRPs have been identified. However, an amplification cycle in which Piwi-mediated cleavage acts as a biogenesis mechanism for secondary piRNAs can serve the same purpose as the RdRP-driven secondary siRNA generation systems in worms, plants and fungi. In fact, the strength of the amplification cycle that we propose is directly tied to the abundance of target RNAs, which may couple piRNA production to the strength of the needed response. Moreover, since the amplification cycle consumes target transposon transcripts as part of its mechanism, post-transcriptional gene silencing mechanisms, within the model that we propose, may be sufficient to explain transposon repression. However, we cannot rule out the possibility that transcriptional silencing may also be triggered by Piwi family RNPs.

The model for transposon silencing that emerges from our studies shows many parallels to adaptive immune systems. The piRNA loci themselves encode a diversity of small RNA fragments that have the potential to recognize invading parasitic genetic elements. Throughout the evolution of Drosophila species, a record of transposon exposure may have been preserved by selection for transposition events into these master control loci, as this is one key mechanism through which control over a specific element can be achieved. Once an element enters a piRNA locus, it can act, in trans, to silencing remaining elements in the genome through the amplification model described above. Evidence has already emerged that X-TAS can act as a transposition hotspot for P-elements (Karpen and Spradling, 1992), raising the possibility the piRNAs clusters in general may attract transposable elements. A comparison of D. melanogaster piRNAs to transposons present in related Drosophilids shows a lack of complementarity when comparisons are made at high stringency. However, when even a few mismatches are permitted, it is clear that piRNA loci might have some limited potential to protect against horizontal transmission of these heterologous elements.

Applicants studied strand asymmetry of piRNAs mapping to all LTR/LINE/IR Transpsons from Drosophila melanogaster and from related Drosophilid species. Analysis was performed and data displayed exactly as described before. A more complete list of melanogaster transposons is studied along with transposons from related Drosophilid species. Heat maps were constructed for matches to consensus at different stringencies (0 mismatches, 3 mismatches, and 5 mismatches). The results show that the existence of a feed-forward amplification loop can be compared to clonal expansion of immune cells with the appropriate specificity following antigen stimulation, leading to a robust and adaptable response.

### Materials and Methods

(a) Antibodies and Immunohistochemistry.

Peptides (Invitrogen) corresponding to the 14-16 N-terminal amino acids of Piwi, Aub and Ago3 (see FIG. 3) were conjugated to KLH and used for inoculation into rabbits for polyclonal antibody production (Covance). Antibodies were affinity purified on a peptide-conjugated resin (Sulfolink, Pierce Biochemicals). For Western blot analysis, primary antibody dilutions of 1:2000 and secondary antibody dilutions of 1:150000 (Amersham; NA9340V) were used. For immunocytochemistry, primary antibody dilutions of 1:500 and secondary antibodies (Alexa 468 conjugated; 1:200) from Molecular Probes were used. DNA staining was done using the TOPRO3 dye from Molecular Probes (1:500). Actin staining was with Rhodamille coupled Phalloidin (Molecular Probes) at 1:100. Ovaries were dissected into ice cold PBS, fixed for 20 min. in 4% Formaldehyde/PBS/0.1% Triton X-100.

(b) Immunoprecipitation of Piwi Family RNP Complexes and Labeling of RNA

Ovaries were dissected into ice cold PBS, flash frozen in liquid nitrogen and stored at −80 degrees. Ovary extract was prepared in Lysis buffer (20 mM HEPES-NaOH pH 7.0, 150 mM NaCl, 2.5 mM MgCl2, 250 mM Sucrose, 0.05% NP40, 0.5% Triton X-100. 1× Roche-Complete EDTA free) using a glass dounce homogenizer. Extracts were cleared by several spins at 14000 rpm. Extracts (10 microgram/microliter) were incubated with primary antibodies (1:50) for 4 h at 4 degrees per ml of extract. Fifteen microliters of Protein-G Sepharose (Roche) were added and mixtures were further incubated for 1 h at 4 degrees. Beads were washed 4 times in lysis buffer. RNA extraction from beads and 5′ labeling of RNAs was done as described in (Aravin et al., 2006)

(c) Small RNA Cloning and Sequencing

RNA extraction from ovaries was done using Trizol (Invitrogen). Small RNA cloning was performed as described in (Pfeffer et al., 2005) with following modifications. To trace ligation products small amount of 5′-labelled immunoprecipitated small RNA were added to non-labeled RNA. Pre-adenylated oligonucleotide (5′ rAppCTGTAGGCACCATCAAT/3ddC/, Linker-1, IDT) was used for ligation of 3′ linker and custom synthesized oligonucleotide (5′ ATCGTrArGrGrCrArCrCrUrGrArUrA, Dharmacon) was used for ligation of 5′ linker. After reverse transcription and amplification with primers that match adapter sequences PCR product was isolated from 3% agarose gel and reamplified using a pair of 454 cloning primers: 5′ primer: GCCTCCCTCGCGCCATCAGATCGTAGGCACCTGATA 3′ primer: GCCTTGCCAGCCCGCTCAGATTGATGGTGCCTACAG The reamplified products were gel-purified and then provided to 454 Life Sciences (Branford, Conn.) for sequencing.

(d) Bioinformatic Analysis of Small RNA Libraries

Sequence extraction and genomic mapping was as described in (Girard et al., 2006). We used the Release 5 assembly of the Drosophila melanogaster genome (http://www.fruitfly.org/sequence/release5genomic.shtml) and the NR database at NCBI to identify all piRNAs mapping 100% to annotated Drosophila melanogaster sequences. The only NR entry which recovered hits not present in the Release 5 sequence (L03284) corresponds to the heterochromatic tip of the X-chromosome, which differs significantly between the sequenced strain and Oregon R, the strain used for our analysis (Abad et al., 2004). Annotation of small RNAs was done using the following databases: Repbase (http://www.girinst.org/) on the Release 5 assembly; Transposable element canonical sequences (http://www.fruitfly.org/p_disrupt/TE.html); Flybase annotations for protein coding and non coding genes (extracted from http://genome.ucsc.edu); and microRNA annotations from Rfam (http://microrna.sanger.ac.uk/sequences). Density analysis of transposons and genes along Release 5 chromosome arms was done by counting all the nucleotides within a 50 Kb window that were annotated as transposons or as exons in Flybase. The window was analyzed at 10 kB increments through the genome.

(e) piRNA Cluster Analysis

All piRNAs except the 10% of reads corresponding to microRNAs, rRNAs, tRNAs, snoRNAs, smRNAs, snRNAs, other ncRNAs and the sense strand of annotated genes were mapped to Release 5 and the telomeric X-TAS repeat L03284. Nucleotides corresponding to the 5′ end of a 100% matched piRNA were weighted according to N/M with N=cloning frequency and M=number of genomic mappings (suppression model). We used a 5 kb sliding window to identify all regions on each chromosome with piRNA densities greater than 1piRNA/kb. Windows within 20 kb of each other were collapsed into clusters, whose start and end coordinates were adjusted to those of the first and last piRNA match. We then removed each cluster that did not contain at least 5 piRNAs that uniquely matched to that cluster.

(f) Analysis of piRNAs Mapping to Transposable Elements

All identified piRNAs were matched to the canonical sequences of Drosophila transposable elements (http://www.fruitfly.org/p_disrupt/TE.html) with high (0 mismatches), medium (3 mismatches) or low (5 mismatches) stringencies and the strand relative to the transposon sense strand was determined. We calculated the ratio of all piRNAs per library that match exclusively to the plus or minus strand and excluded those that matched to both (for example in IR elements). For the relative density of piRNAs on transposable elements, the fraction of piRNAs mapping to a specific element as compared to all piRNAs matching to any element was determined. Each library was analyzed individually, as cross-library comparisons are not possible. The presented data incorporates the cloning frequency of individual piRNAs. Very similar results were obtained if cloning frequency was not considered.

(g) 10-nt Offset Analysis

For this analysis, which uses genomic mapping coordinates of piRNAs, all genomic positions corresponding to a 100% matching piRNA 5′ end were weighted according to the suppression model (see above). The average “neighborhood” of sequences on the antisense strand was determined as the sum of 5′ ends in the suppression model (see above) in respect to the 5′ position of the sense strand piRNA. We determined the fraction of piRNAs that had a reverse complement sequence match between their 5′ most 10 mers and other 10 mers in the dataset depending on the other 10 mers position in the respective sequences. To show the specificity of the 10 mer overlaps at the 5′ ends, we repeated the analysis for 10 mers from positions 2-11. To investigate the library distribution of piRNA 10 mer overlapping pairs, we determined the fraction of all piRNAs in each library that has a partner piRNA in the other libraries. We did this with and without taking cloning frequency into account and repeated the analysis for the 10 mers from 2-11 as a control. We finally tested for a correlation between the cloning frequency and the tendency to have a 10 mer partner. We sorted all piRNAs in each library according to their cloning frequency and determined the fraction of piRNAs with 10 mer partners in bins, each containing 10% of all reads.

(h) Nucleotide Bias of piRNAs

We determined position dependent nucleotide biases for each library by their log-odds score relative to library specific background nucleotide frequencies. Pictograms were made using perl svg and bioperl libraries.

### LITERATURE CITED

Abad, J. P., De Pablos, B., Osoegawa, K., De Jong, P. J., Martin-Gallardo, A., and Villasante, A. (2004). Genomic analysis of Drosophila melanogaster telomeres: full-length copies of HeT-A and TART elements at telomeres. Mol Biol Evol 21, 1613-1619.
Aravin, A., Gaidatzis, D., Pfeffer, S., Lagos-Quintana, M., Landgraf, P., Iovino, N., Morris, P., Brownstein, M. J., Kuramochi-Miyagawa, S., Nakano, T., et al. (2006). A novel class of small RNAs bind to MILI protein in mouse testes. Nature 442, 203-207.
Aravin, A. A., Lagos-Quintana, M., Yalcin, A., Zavolan, M., Marks, D., Snyder, B., Gaasterland, T., Meyer, J., and Tuschl, T. (2003). The small RNA profile during Drosophila melanogaster development. Dev Cell 5, 337-350.
Aravin, A. A., Naumova, N. M., Tulin, A. V., Vagin, V. V., Rozovsky, Y. M., and Gvozdev, V. A. (2001). Double-stranded RNA-mediated silencing of genomic tandem repeats and transposable elements in the D. melanogaster germline. Curr Biol 11, 1017-1027.
Biemont, C., Ronsseray, S., Anxolabehere, D., Izaabel, H., and Gautier, C. (1990). Localization of P elements, copy number regulation, and cytotype determination in Drosophila melanogaster. Genet Res 56, 3-14.
Bingham, P. M., Kidwell, M. G., and Rubin, G. M. (1982). The molecular basis of P-M hybrid dysgenesis: the role of the P element, a P-strain-specific transposon family. Cell 29, 995-1004.
Blumenstiel, J. P., and Hartl, D. L. (2005). Evidence for maternally transmitted small interfering RNA in the repression of transposition in Drosophila virilis. Proc Natl Acad Sci USA 102, 15965-15970.
Bregliano, J. C., Picard, G., Bucheton, A., Pelisson, A., Lavige, J. M., and L'Heritier, P. (1980). Hybrid dysgenesis in Drosophila melanogaster. Science 207, 606-611.
Brookfield, J. F. (2005). The ecology of the genome—mobile DNA elements and their hosts. Nat Rev Genet. 6, 128-136.
Bucheton, A. (1990). I transposable elements and I-R hybrid dysgenesis in Drosophila. Trends Genet. 6, 16-21.
Bucheton, A. (1995). The relationship between the flamenco gene and gypsy in Drosophila: how to tame a retrovirus. Trends Genet II, 349-353.
Bucheton, A., Paro, R., Sang, H. M., Pelisson, A., and Finnegan, D. J. (1984). The molecular basis of 1-R hybrid dysgenesis in Drosophila melanogaster: identification, cloning, and properties of the I factor. Cell 38, 153-163.
Carmell, M. A., Xuan, Z., Zhang, M. Q., and Hannon, G. J. (2002). The Argonaute family: tentacles that reach into RNAi, developmental control, stem cell maintenance, and tumorigenesis. Genes Dev 16, 2733-2742.
Castro, J. P., and Carareto, C. M. (2004). Drosophila melanogaster P transposable elements: mechanisms of transposition and regulation. Genetica 121, 107-118.
Chen, P. Y., Manning a, H., Slanchev, K., Chien, M., Russo, J. J., Ju, J., Sheridan, R., John, B., Marks, D. S., Gaidatzis, D., et al. (2005). The developmental miRNA profiles of zebrafish as determined by small RNA cloning. Genes Dev 19, 1288-1293.
Cox, D. N., Chao, A., Baker, J., Chang, L., Qiao, D., and Lin, H. (1998). A novel class of evolutionarily conserved genes defined by piwi are essential for stem cell self-renewal. Genes Dev 12, 3715-3727.
Cox, D. N., Chao, A., and Lin, H. (2000). piwi encodes a nucleoplasmic factor whose activity modulates the number and division rate of germline stem cells. Development 127, 503-514.
Deng, W., and Lin, H. (2002). miwi, a murine homolog of piwi, encodes a cytoplasmic protein essential for spermatogenesis. Dev Cell 2, 819-830.
Desset, S., Meignin, C., Dastugue, B., and Vaury, C. (2003). COM, a heterochromatic locus governing the control of independent endogenous retroviruses from Drosophila melanogaster. Genetics 164, 501-509.
Engels, W. R., and Preston, C. R. (1979). Hybrid dysgenesis in Drosophila melanogaster: the biology of female and male sterility. Genetics 92, 161-174.
Findley, S. D., Tamanaha, M., Clegg, N. J., and Ruohola-Baker, H. (2003). Maelstrom, a Drosophila spindle-class gene, encodes a protein that colocalizes with Vasa and RDE1/AGO1 homolog, Aubergine, in nuage. Development 130, 859-871.
Girard, A., Sachidanandam, R., Hannon, G. J., and Carmell, M. A. (2006). A germline-specific class of small RNAs binds mammalian Piwi proteins. Nature 442, 199-202.
Grivna, S. T., Pyhtila, B., and Lin, H. (2006). MIWI associates with translational machinery and PIWI-interacting RNAs (piRNAs) in regulating spermatogenesis. Proc Natl Acad Sci USA 103, 13415-13420.
Hamilton, A. J., and Baulcombe, D. C. (1999). A species of small antisense RNA in posttranscriptional gene silencing in plants. Science 286, 950-952.
Han, J. S., and Boeke, J. D. (2005). LINE-1 retrotransposons: modulators of quantity and quality of mammalian gene expression? Bioessays 27, 775-784.
Harris, A. N., and Macdonald, P. M. (2001). Aubergine encodes a Drosophila polar granule component required for pole cell formation and related to eIF2C. Development 128, 2823-2832.
Herr, A. J. (2005). Pathways through the small RNA world of plants. FEBS Lett 579, 5879-5888. Hoskins, R. A., Smith, C. D., Carlson, J. W., Carvalho, A. B., Halpern, A.,
Kaminker, J. S., Kennedy, C., Mungall, C. J., Sullivan, B. A., Sutton, G. G., et al. (2002). Heterochromatic sequences in a Drosophila whole-genome shotgun assembly. Genome Biol 3, RESEARCH0085.
Kalmykova, A. I., Klenov, M. S., and Gvozdev, V. A. (2005). Argonaute protein PIWI controls mobilization of retrotransposons in the Drosophila male germline. Nucleic Acids Res 33, 2052-2059.
Karpen, G., and Spradling, A. (1992). Analysis of subtelomeric heterochromatin in the Drosophila minichromosome Dp1187 by single P element insertional mutagenesis. Genetics 132, 737-753.
Kazazian, H. H., Jr. (2004). Mobile elements: drivers of genome evolution. Science 303, 1626-1632.
Ketting, R. F., Haverkamp, T. H., van Luenen, H. G., and Plasterk, R. H. (1999). Mut-7 of C. elegans, required for transposon silencing and RNA interference, is a homolog of Werner syndrome helicase and RNaseD. Cell 99, 133-141.
Kidwell, M. G., Kidwell, J. F., and Sved, J. A. (1977). Hybrid Dysgenesis in Drosophila melanogaster: A Syndrome of Aberrant Traits Including Mutation, Sterility and Male Recombination. Genetics 86, 813-833.
Kuramochi-Miyagawa, S., Kimura, T., Ijiri, T. W., Isobe, T., Asada, N., Fujita, Y., Ikawa, M., Iwai, N., Okabe, M., Deng, W., et al. (2004). Mili, a mammalian member of piwi family gene, is essential for spermatogenesis. Development 131, 839-849.
Lau, N. C., Seto, A. G., Kim, J., Kuramochi-Miyagawa, S., Nakano, T., Bartel, D. P., and Kingston, R. E. (2006). Characterization of the piRNA complex from rat testes. Science 313, 363-367.
Lin, H., and Spradling, A. C. (1997). A novel group of pumilio mutations affects the asymmetric division of germline stem cells in the Drosophila ovary. Development 124, 2463-2476.
Liu, J., Cannell, M. A., Rivas, F. V., Marsden, C. G., Thomson, J. M., Song, J. J., Hammond, S. M., Joshua-Tor, L., and Hannon, G. J. (2004). Argonaute2 is the catalytic engine of mammalian RNAi. Science 305, 1437-1441.
Marin, L., Lehmann, M., Nouaud, D., Izaabel, H., Anxolabehere, D., and Ronsseray, S. (2000). P-Element repression in Drosophila melanogaster by a naturally occurring defective telomeric P copy. Genetics 155, 1841-1854.
Martienssen, R. A., Zaratiegui, M., and Goto, D. B. (2005). RNA interference and heterochromatin in the fission yeast Schizosaccharomyces pombe. Trends Genet. 21, 450-456.
Megosh, H. B., Cox, D. N., Campbell, C., and Lin, H. (2006). The Role of PIWI and the miRNA Machinery in Drosophila Germline Determination. Curr Biol 16, 1884-1894.
Misra, S., and Rio, D. C. (1990). Cytotype control of Drosophila P element transposition: the 66 kd protein is a repressor of transposase activity. Cell 62, 269-284.
Pal-Bhadra, M., Bhadra, U., and Birchier, J. A. (1997). Cosuppression in Drosophila: gene silencing of Alcohol dehydrogenase by white-Adh transgenes is Polycomb dependent. Cell 90, 479-490.
Pal-Bhadra, M., Bhadra, U., and Birchler, J. A. (2002). RNAi related mechanisms affect both transcriptional and posttranscriptional transgene silencing in Drosophila. Mol Cell 9, 315-327.
Pal-Bhadra, M., Leibovitch, B. A., Gandhi, S. G., Rao, M., Bhadra, U., Birchier, J. A., and Elgin, S. C. (2004). Heterochromatic silencing and HP1 localization in Drosophila are dependent on the RNAi machinery. Science 303, 669-672.
Pardue, M. L., and DeBaryshe, P. G. (2003). Retrotransposons provide an evolutionarily robust non-telomerase mechanism to maintain telomeres. Annu Rev Genet. 37, 485-511. Pelisson, A. (1981). The I-R system of hybrid dysgenesis in Drosophila melanogaster: are I factor insertions responsible for the mutator effect of the I-R interaction? Mol Gen Genet 183, 123-129. Pelisson, A., and Bregliano, J. C. (1987). Evidence for rapid limitation of the I element copy number in a genome submitted to several generations of 1-R hybrid dysgenesis in Drosophila melanogaster. Mol Gen Genet. 207, 306-313.
Pelisson, A., Song, S. U., Prud'homme, N., Smith, P. A., Bucheton, A., and Corces, V. G. (1994). Gypsy transposition correlates with the production of a retroviral envelope-like protein under the tissue-specific control of the Drosophila flamenco gene. Embo J 13, 4401-4411.
Petrov, D. A., Schutzman, J. L., Hartl, D. L., and Lozovskaya, E. R. (1995). Diverse transposable elements are mobilized in hybrid dysgenesis in Drosophila virilis. Proc Natl Acad Sci USA 92, 8050-8054.
Pfeffer, S., Sewer, A., Lagos-Quintana, M., Sheridan, R., Sander, C., Grasser, F. A., van Dyk, L. F., Ho, C. K., Shuman, S., Chien, M., et al. (2005). Identification of microRNAs of the herpesvirus family. Nat Methods 2, 269-276.
Prud'homme, N., Gans, M., Masson, M., Terzian, C., and Bucheton, A. (1995). Flamenco, a gene controlling the gypsy retrovirus of Drosophila melanogaster. Genetics 139, 697-711.
Reiss, D., Josse, T., Anxolabehere, D., and Ronsseray, S. (2004). aubergine mutations in Drosophila melanogaster impair P cytotype determination by telomeric P elements inserted in heterochromatin. Mol Genet Genomics 272, 336-343.
Rivas, F. V., Tolia, N. H., Song, J. J., Aragon, J. P., Liu, J., Hannon, G. J., and Joshua-Tor, L. (2005). Purified Argonaute2 and an siRNA form recombinant human RISC. Nat Struct Mol Biol 12, 340-349.
Robert, V., Prud'homme, N., Kim, A., Bucheton, A., and Pelisson, A. (2001). Characterization of the flamenco region of the Drosophila melanogaster genome. Genetics 158, 701-713.
Robertson, H. M., and Engels, W. R. (1989). Modified P elements that mimic the P cytotype in Drosophila melanogaster. Genetics 123, 815-824.
Roche, S. E., and Rio, D. C. (1998). Trans-silencing by P elements inserted in subtelomeric heterochromatin involves the Drosophila Polycomb group gene, Enhancer of zeste. Genetics 149, 1839-1855.
Ronsseray, S., Lehmann, M., and Anxolabehere, D. (1991). The maternally inherited regulation of P elements in Drosophila melanogaster can be elicited by two P copies at cytological site 1A on the X chromosome. Genetics 129, 501-512.
Ronsseray, S., Marin, L., Lehmann, M., and Anxolabehere, D. (1998). Repression of hybrid dysgenesis in Drosophila melanogaster by combinations of telomeric P-element reporters and naturally occurring P elements. Genetics 149, 1857-1866.
Rubin, G. M., Kidwell, M. G., and Bingham, P. M. (1982). The molecular basis of P-M hybrid dysgenesis: the nature of induced mutations. Cell 29, 987-994.
Saito, K., Nishida, K. M., Mori, T., Kawamura, Y., Miyoshi, K., Nagami, T., Siomi, H., and Siomi, M. C. (2006). Specific association of Piwi with rasiRNAs derived from retrotransposon and heterochromatic regions in the Drosophila genome. Genes Dev 20, 2214-2222.
Sarot, E., Payen-Groschene, G., Bucheton, A., and Pelisson, A. (2004). Evidence for a piwi-dependent RNA silencing of the gypsy endogenous retrovirus by the Drosophila melanogaster flamenco gene. Genetics 166, 1313-1321.
Savitsky, M., Kwon, D., Georgiev, P., Kalmykova, A., and Gvozdev, V. (2006). Telomere elongation is under the control of the RNAi-based mechanism in the Drosophila germline. Genes Dev 20, 345-354.
Sijen, T., Fleenor, J., Simmer, F., Thijssen, K. L., Parrish, S., Timmons, L., Plasterk, R. H., and Fire, A. (2001). On the Role of RNA Amplification in dsRNATriggered Gene Silencing. Cell 107, 465-476.
Simmons, M. J., Johnson, N. A., Fahey, T. M., Nellett, S. M., and Raymond, J. D. (1980). High mutability in male hybrids of Drosophila melanogaster. Genetics 96, 479-480.
Smyth, D. R. (1997). Gene silencing: cosuppression at a distance. Curr Biol 7, R793-795.
Stuart, J. R., Haley, K. J., Swedzinski, D., Lockner, S., Kocian, P. E., Merriman, P. J., and Simmons, M. J. (2002). Telomeric P elements associated with cytotype regulation of the P transposon family in Drosophila melanogaster. Genetics 162, 1641-1654.
Tabara, H., Sarkissian, M., Kelly, W. G., Fleenor, J., Grishok, A., Timmons, L., Fire, A., and Mello, C. C. (1999). The rde-1 gene, RNA interference, and transposon silencing in C. elegans. Cell 99, 123-132.
Vagin, V. V., Sigova, A., Li, C., Seitz, H., Gvozdev, V., and Zamore, P. D. (2006). A distinct small RNA pathway silences selfish genetic elements in the germline. Science 313, 320-324.
Williams, R. W., and Rubin, G. M. (2002). ARGONAUTE1 is required for efficient RNA interference in Drosophila embryos. Proc Natl Acad Sci USA 99, 6889-6894.

TABLE I
Top 15 piRNA-producing loci in D. melanogaster genome

| | | | | Number of | Potential | piRNA strand |
|---|---|---|---|---|---|---|
| | | | Transposon | uniquely- | piRNA, | distribution |
| | Chrom. | | content (+/− | mapped | number | (+/− |
| Number | band | Genomic position | strand, %) | piRNAs | (%) | strand, %) |
| 1 | 42A-B | arm_2R, | 37.8/32.2 | 1686 | 15102 | 48.6/51.4 |
| | | 2144349-2386719 | | | (30.1%) | |
| 2 | 20A | arm_X, | 0.2/78.4 | 986 | 8621 | 100/0 |
| | | 21392175-21431907 | | | (17.2%) | |
| 3 | 102E | arm_4, | 5.8/82.9 | 684 | 2519 | 22.5/77.5 |
| | | 1258473-1348320 | | | (5%) | |
| 4 | 1A | — | 0/2.9 | 484 | 1306 | 4.44/55.6 |
| | | | | | (2.6%) | |
| 5 | 38C | arm_2L, | 23.4/63.6 | 482 | 1851 | 54.1/45.9 |
| | | 20148259-20227581 | | | (3.7%) | |
| 6 | 80E-F | arm_3L, | 28.9/37.4 | 228 | 1455 | 63.8/36.2 |
| | | 23273964-23314199 | | | (2.9%) | |
| 7 | — | ArmU, | 22.9/20.5 | 180 | 1097 | 62.1/37.9 |
| | | 4013706-4088786 | | | (2.2%) | |
| 8 | 20A-B | arm_X, | 12.8/74.2 | 170 | 6684 | 98.5/1.5 |
| | | 21505666-21687255 | | | (13.3%) | |
| 9 | 20B | arm_X, | 23.5/55.2 | 155 | 2187 | 62.7/37.3 |
| | | 21759393-21844063 | | | (4.4%) | |
| 10 | — | ArmU, | 28.3/35.2 | 146 | 4970 | 52.4/47.6 |
| | | 5689564-5779439 | | | (9.9%) | |
| 11 | 100E | arm_3R, | 10.7/3.5 | 107 | 932 | 0/100 |
| | | 27895169-27905030 | | | (1.9%) | |
| 12 | — | 3LHet, | 27.6/38.8 | 102 | 4789 | 51.1/48.9 |
| | | 1402377-1557939 | | | (9.5%) | |
| 13 | — | 3LHet, | 35.8/33.9 | 92 | 7607 | 35.7/64.3 |
| | | 2011004-2230834 | | | (15.2%) | |
| 14 | — | ArmU, | 33.1/29.3 | 91 | 7167 | 58.7/41.3 |
| | | 7498151-7588549 | | | (14.3%) | |
| 15 | — | ArmU, | 43.5/33.2 | 76 | 6743 | 43.6/56.4 |
| | | 923516-1066801 | | | (13.4%) | |

piRNA-producing loci were sorted by the number of piRNA clones that are unambiguously derived from corresponding locus (column 5). Genomic positions of piRNA producing loci are given according to Release 5 assembly of D. melanogaster genome (Flybase). For cluster 4, located in the telomeric heterochromatin of X chromosome (position 1A), the corresponding sequence is absent in the current genomic assembly. Positions of piRNA-producing regions on the polytene chromosome map (column 2) are determined by mapping genomic positions to Release 4.3 genome assembly and extraction of corresponding cytological band annotation according to the FlyBase Genome Browser. An assignment of cytological band proved impossible for some heterochromatic sequences (cluster 7 and 12-15). The percentage of transposon-derived sequences on the plus and minus strands (column 4) was determined as described in Materials and Methods. To calculate the number of piRNA clones that are potentially derived from each region (column 6) all sequences that match the genomic sequence of the region with zero mismatches were considered. To calculate the strand distribution of piRNAs (column 7) sequences that match to the genome at a unique site were considered.

### Example VII

### Developmentally Regulated piRNA Clusters Implicate MILI in Transposon Control

Nearly half of the mammalian genome is composed of repeated sequences. In Drosophila, Piwi proteins exert control over transposons. However, mammalian Piwi proteins, MIWI and MILI, partner with Piwi-interacting RNAs (piRNAs) that are depleted of repeat sequences, which raises questions about a role for mammalian Piwi's in transposon control.

This example, partly based on a search for murine small RNAs that might program Piwi proteins for transposon suppression, demonstrates the presence of a developmentally regulated piRNA loci in mammal, some of which resemble transposon master control loci of Drosophila. Applicants also found evidence of an adaptive amplification loop in which MILI catalyzes the formation of piRNA 5′ ends. Mili mutants derepress LINE-1 (L1) and intracisternal A particle and lose DNA methylation of L1 elements, demonstrating an evolutionarily conserved role for PIWI proteins in transposon suppression.

Applicants showed that MILI associates with distinct small RNA populations during spermatogenesis. Specifically, MILI-associated RNAs were analyzed from testes of 8-, 10-, and 12-day-old and adult mice with proper control. Testes RNA or RNA from MILI immunoprecipitates (IP) from mice of indicated ages was analyzed by Northern blotting for a prepachytene piRNA, a pachytene piRNA, or let-7 (residual let-7 signal observed). Northern hybridization of RNA isolated from P10 testes of WT mice and Mili-heterozygous and Milihomozygous mutants were determined.

Results show that known mouse piRNAs are not expressed until spermatocytes first enter mid-prophase (pachytene stage) at ˜14 days after birth (P14). However, Mili expression begins in primordial germ cells at embryonic day 12.5, and transposons, such as L1, can be expressed in both premeiotic and meiotic germ cells. We therefore probed a connection between Mili and transposon control by examining MILI-bound small RNAs in early stage spermatocytes. Notably, MILI-associated RNAs could be detected at all developmental time points tested (see FIG. 1 and FIG. S1 of Aravin et al., Science 316: 744-747, 2007, incorporated by reference). Northern blotting revealed that pre-pachytene piRNAs join MILI before pachytene piRNAs become expressed at P14. The appearance of pre-pachytene piRNAs was MILI-dependent, suggesting a requirement for this protein in either their biogenesis or stability. These results raised the possibility that MILI might be programmed by distinct piRNA populations at different stages of germ cell development.

To characterize pre-pachytene piRNAs, Applicants isolated MILI complexes from P10 testes and deeply sequenced their constituent small RNAs. Like pachytene populations, pre-pachytene piRNAs were quite diverse, with 84% being cloned only once. The majority of both pre-pachytene (66.8%) and pachytene (82.9%) piRNAs map to single genomic locations. However, a substantial fraction (20.1%) of pre-pachytene piRNAs had more than 10 genomic matches, as compared to 1.6% for pachytene piRNAs.

Annotation of pre-pachytene piRNAs revealed three major classes. The largest (35%) corresponded to repeats, with most matching short interspersed elements (SINEs) (49%), long interspersed elements (LINEs) (15.8%), and long terminal repeat (LTR) retrotransposons (33.8%). Although pachytene piRNAs also match repeats (17%), the majority (>80%) map uniquely in the genome, with only 1.8% mapping more than 1000 times (FIG. S2 of Aravin et al., Science 316: 744-747, 2007, incorporated by reference). In contrast, 22% of repeat-derived pre-pachytene piRNAs map more than 1000 times and correspond closely to consensus sequences for SINE B1, LINE L1, and IAP retrotransposons (FIG. S2 of Aravin et al., Science 316: 744-747, 2007, incorporated by reference). A second abundant class of pre-pachytene piRNAs (29%) matched genic sequences, including both exons (22%) and introns (7%). A third class matched sequences without any annotation (28%). All three major classes shared signature piRNA characteristics, including a preference for a uridine (U) at their 5′ end (>80%). Pachytene piRNAs derive from relatively few extended genomic regions, with hundreds to thousands of different species encoded from a single genomic strand. Cluster analysis of pre-pachytene piRNAs yielded 909 loci, covering 0.2% of the mouse genome (5.3 megabases; table S1). Pachytene and pre-pachytene clusters show little overlap (FIGS. 2B and 2C, and table S1 of Aravin et al., Science 316: 744-747, 2007, incorporated by reference). Overall, pachytene clusters were larger, and each produced a greater fraction of the piRNA population than early clusters, which average 5.8 kb in size. Only 56.5% of uniquely mapped pre-pachytene piRNAs can be attributed to clusters, as compared to 95.5% in pachytene piRNA populations. Considered together, these results demonstrate that prepachytene and pachytene piRNAs are derived from different genomic locations, with prepachytene piRNAs being produced from a broader set of loci.

The 28% of pre-pachytene piRNAs that correspond to protein coding genes were concentrated in 3′ untranslated regions (3′UTRs) (FIG. S3 of Aravin et al., Science 316: 744-747, 2007, incorporated by reference) and showed a strong bias for certain loci, with 8% of the total coming from only 10 genes. These were invariably derived from the sense strand.

Clusters that are rich in transposon sequences were among the most prominent, as judged by either their size or the number of piRNAs that they generate. Two of these were the largest prepachytene clusters (97 and 79 kb, respectively). Although uniquely mapping piRNAs were derived largely from one genomic strand, the mixed orientations of transposable elements within clusters led to the production of both sense and antisense piRNAs. As is observed in Drosophila, repeat-rich mouse piRNA clusters typically contained multiple element types, many of which comprise damaged or fragmented copies. In many repeat-rich clusters, the orientation of most elements was similar. For example, similarly oriented elements in the two longest clusters (FIG. 2D and table S1 of Aravin et al., Science 316: 744-747, 2007, incorporated by reference) resulted in the production of mainly antisense piRNAs, similar to the flamenco piRNA locus in Drosophila.

We examined the possibility that prepachytene piRNAs might program MILI to repress transposon activity, and found that Mili regulates L1 and IAP elements. Specifically, quantitative RT-PCR for IAP and L1 expression in testes from WT or Mili-null mice were performed. Expression was assessed at P10 and P14. DNA was isolated from the tails or testes of Mili^{+/+}, Mili^{+/-}, or Mili^{-/-} animals; digested with either a methylation-insensitive [Msp I(M)] or a methylation-sensitive [Hpa II (H)] restriction enzyme; and used in a Southern blot with a probe from the LINE-1 5′UTR. Applicants observed DNA bands arising from loss of methylation in the Mili-null animals. Bisulfite sequencing of the first 150 bases of a specific L1 element was done in Mili^{+/-} or Mili^{-/-} animals.

These results show that Mili mutation had substantial effects on L1 and IAP expression, with each increasing its levels by a factor of at least 5 to 10. These studies were carried out at P10 and P14, before an overt Mili phenotype becomes apparent.

Although posttranscriptional mechanisms likely contribute to silencing, CpG methylation is critical for transposon repression in mammals. Both analysis with methylationsensitive restriction enzymes and bisulfite DNA sequencing revealed substantial demethylation of L1 elements in Mili-mutant testes. In the latter case, the ˜50% of L1 sequences that remain methylated in the mutant are likely derived from the somatic compartment.

Considered together, our data suggest that pre-pachytene piRNAs might help to guide methylation of L1 elements.

In Drosophila, Piwi-mediated cleavage promotes the formation of secondary piRNAs. This allows active transposons and piRNA clusters to participate in a feed-forward loop that both degrades transposon mRNAs and amplifies silencing. The presence of both sense and antisense piRNAs from mammalian transposable elements creates the potential for engagement of a similar amplification cycle. This cycle creates two tell-tale features. First, because Piwi proteins cleave targets opposite nucleotides 10 and 11 of the guide, piRNAs generated within the loop overlap their partners by precisely 10 nucleotides.

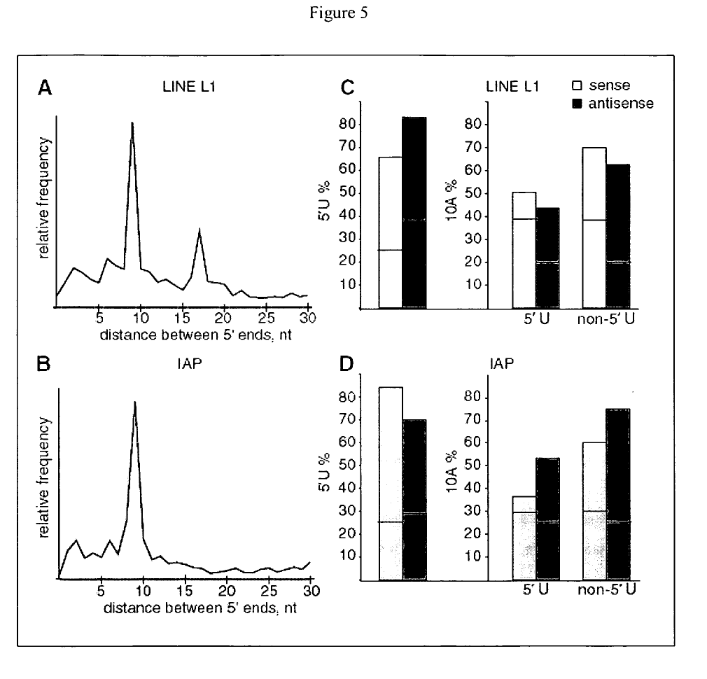

As predicted, we observed enrichment for piRNAs corresponding to L1 and IAP retrotransposons, in which the 5′ ends of sense and antisense partners are separated by precisely 10 nucleotides (FIGS. 5A and 5B). Second, because most piRNAs begin with a U, piRNAs produced by Piwi-mediated cleavage are enriched for adenine (A) at position 10. This bias was prevalent in L1- and TAP-derived piRNAs (the fraction of A at position 10 (10A) in FIGS. 5C and 5D). For piRNAs to be cleavage competent and active in the amplification cycle, they must retain a high degree of complementarity to their targets (FIG. S4 of Aravin et al., Science 316: 744-747, 2007, incorporated by reference). Consistent with this hypothesis, piRNAs that map uniquely in the genome have a lower bias for 10A (e.g., 38.7% for non-5′U piRNAs matching LTR-containing retrotransposons) than do piRNAs with many (e.g., >11000) genomic matches (61.5%).

Our results suggest a conserved pathway through which a developmentally regulated cascade of piRNA clusters programs Piwi proteins to repress transposons in mammals.

One key difference between transposon control in Drosophila and mammals is the role of cytosine methylation in maintaining stable repression. In plants, it is well established that small RNAs can guide methylation of complementary sequences. The observations that Miwi2 and Mili mutations strongly affect methylation of L1 elements and that MILI binds L1-targeted small RNAs suggest that mammals may also harbor an RNA-dependent DNA methylation pathway.

### REFERENCES CITED FOR EXAMPLE VII

1. N. C. Lau et al., Science 313, 363 (2006).
2. S. T. Grivna, E. Beyret, Z. Wang, H. Lin, Genes Dev. 20, 1709 (2006).
3. A. Aravin et al., Nature 442, 203 (2006).
4. A. Girard, R. Sachidanandam, G. J. Hannon, M. A. Carmell, Nature 442, 199 (2006).
5. S. Kuramochi-Miyagawa et al., Mech. Dev. 108, 121 (2001).
6. S. Kuramochi-Miyagawa et al., Development 131, 839 (2004).
7. H. H. Kazazian Jr., Science 303, 1626 (2004).
8. D. Branciforte, S. L. Martin, Mol. Cell. Biol. 14, 2584 (1994).
9. J. Brennecke et al., Cell 128, 1089 (2007).
10. A. Bucheton, Trends Genet. 11, 349 (1995).
11. G. Liang et al., Mol. Cell Biol. 22, 480 (2002).
12. F. Gaudet et al., Mol. Cell Biol. 24, 1640 (2004).
13. Z. Lippman, B. May, C. Yordan, T. Singer, R. Martienssen, PLoS Biol. 1, E67 (2003).
14. D. Bourc'his, T. H. Bestor, Nature 431, 96 (2004).
15. J. A. Yoder, C. P. Walsh, T. H. Bestor, Trends Genet. 13,335 (1997).
16. T. H. Bestor, D. Bourc'his, Cold Spring Harbor Symp. Quant. Biol. 69, 381 (2004).
17. L. S. Gunawardane et al., Science 315, 1587 (2007).
18. W. Aufsatz, M. F. Mette, J. van der Winden, A. J. Matzke, M. Matzke, Proc. Natl. Acad. Sci. U.S.A. 99 (suppl. 4), 16499 (2002).
19. O. Mathieu, J. Bender, J. Cell Sci. 117, 4881 (2004).
20. M. A. Carmell et al., Dev. Cell 12, 503 (2007).
21. piRNA sequences are available in the Gene Expression Omnibus (GEO) database (accession # GSE7414, all are incorporated herein by reference).

### Example VIII

### MIWI2 is Essential for Spermatogenesis and Repression of Transposons in the Mouse Male Germline

In animals, the Argonaute superfamily segregates into two clades. The Argonaute clade acts in RNAi and in microRNA-mediated gene regulation in partnership with 21-22 nt RNAs. The Piwi clade, and their 26-30 nt piRNA partners, play important roles in germline cells and transposon suppression. For example, in mice, two Piwi-family members have essential roles in spermatogenesis. Here, Applicants provide evidence to show that, disrupting the gene encoding the third family member, MIWI2, causes a meiotic-progression defect in early prophase of meiosis I, and a marked and progressive loss of germ cells with age. These phenotypes suggests inappropriate activation of transposable elements in Miwi2 mutants. These data suggest a conserved function for Piwi-clade proteins in the control of transposons in the germline.

Argonaute proteins lie at the heart of RISC, the RNAi effector complex, and are defined by the presence of two domains, PAZ and Piwi. Phylogenetic analysis of PAZ- and Piwi-containing proteins in animals suggests that they form two distinct clades, with several orphans. One clade is most similar to Arabidopsis ARGONAUTE1. Proteins of this class use siRNAs and microRNAs as sequence-specific guides for the selection of silencing targets. The second clade is more similar to Drosophila PIWI. Like Argonautes, Piwi proteins have been implicated in gene-silencing events, both transcriptional and post-transcriptional.

Piwi-clade proteins have been best studied in the fly, which possesses three such proteins: PIWI, AUBERGINE, and AGO3. Until recently, evidence for the involvement of Piwi proteins in gene silencing was mainly genetic. The first biochemical insight into the biological role of Piwi family proteins was the observation that both PIWI and AUBERGINE exist in complexes with repeat-associated siRNAs (rasiRNAs) (Saito et al., 2006; Vagin et al., 2006).

RasiRNAs were first described in Drosophila as 24-26 nt, small RNAs corresponding to repetitive elements, including transposons (Aravin et al., 2001, 2003). The interaction between Piwi proteins and rasiRNAs dovetails nicely with the observation that, in Drosophila, both piwi and aubergine are important for the silencing of repetitive elements.

Mutations in Piwi-family genes cause defects in germline development in multiple organisms. For example, in flies, piwi is necessary for self-renewing divisions of germline stem cells in both males and females (Cox et al., 1998; Lin and Spradling, 1997). Mutations in aubergine cause male sterility and maternal effect lethality (Schmidt et al., 1999). The male sterility is directly attributable to the failure to silence the repetitive stellate locus. Mutant testes also suffer from meiotic nondisjunctionl of sex chromosomes and autosomes (Schmidt et al., 1999). A recent study indicates that the sterility observed in female flies bearing mutations in Piwi-family proteins is also likely to result, at least in part, from the deleterious effects of transposon activation (Brennecke et al., 2007).

As is seen in other organisms, the expression of the three murine Piwi proteins, MIWI (PIWIL1), MILI (PIWIL2), and MIWI2 (PIWIL4), is largely germline restricted (Kuramochi-Miyagawa et al., 2001; Sasaki et al., 2003). Thus far, MIWI and MILI have been characterized in some detail, with mice bearing targeted mutations in either Miwi (Deng and Lin, 2002) or Mili (Kuramochi-Miyagawa et al., 2004) being male sterile. Although both MIWI and MILI are involved in regulation of spermatogenesis, loss of either protein produces distinct defects that are thematically different from those seen upon mutation of Drosophila piwi. Based upon their expression patterns and the reported phenotypes of mutants lacking each protein, the most parsimonious model is that both MIWI and MILI perform roles essential for the meiotic process. So far, no mammalian Piwi protein has a demonstrated role in stem cell maintenance as proposed for Drosophila PIWI. This raised the possibility that any role for mammalian Piwi proteins in stem cell maintenance might reside in the third family member, MIWI2.

Despite the presence of conserved RNA-binding motifs and an expectation that mammalian Piwi proteins might be involved in RNA-induced silencing mechanisms, no interaction was described for these proteins with siRNAs or miRNAs. Recently, Applicants identified small RNA binding partners for Piwi proteins in the male germline, designated as piRNAs (Piwi-interacting RNAs) (Aravin et al., 2006; Girard et al., 2006; Grivna et al., 2006; Lau et al., 2006; Watanabe et al., 2006). piRNAs show distinctive localization patterns in the genome. They are predominantly grouped into 20-90 kb genomic regions, wherein numerous small RNAs are produced from only one genomic strand. Most piRNAs match the genome at unique sites, and less than 20% match repetitive elements. piRNAs become abundant in germ cells around the pachytene stage of prophase of meiosis 1, but they may be present at lower levels during earlier stages. Unlike microRNAs, individual piRNAs are not conserved.

To investigate the role of MIWI2 in gametogenesis, Applicants disrupted the gene encoding this third mouse Piwi-family member. We find that Miwi2 mutants have two discrete defects in spermatogenesis. The first is a specific meiotic block in prophase of meiosis I that exhibits distinctive morphological features. This is followed by a progressive loss of germ cells from the seminiferous tubules. These phenotypes, and the fact that Miwi2 is expressed both in germline and somatic compartments, highlight similarities between MIWI2 and Drosophila PIWI. In this regard, we find that disruption of Miwi2 also interferes with transposon silencing in the male germline.

We used an insertional mutagenesis strategy to disrupt the Miwi2 gene and generate a mutant Miwi2 Allele. The insertion duplicates exons 9-12. Approximately 10 kb of vector sequence is also inserted into the gene. Wild-type, heterozygous, and homozygous mutant animals were identified by Southern blot analysis using an internal probe. The targeted allele gives two signals, both distinct from wild-type, because the probe is within the duplicated region.

The allele that we created contains a 10 kb segment of vector sequence following Miwi2 exon 12. Downstream of the vector insertion, the genomic region encompassing exons 9-12 is duplicated. This is predicted to insert multiple in-frame stop codons and to produce a nonfunctional allele. When primers downstream of the insertion are used, quantitative RT-PCR indicates that Miwi2 transcripts are essentially undetectable in homozygous mutant animals at 10 days postpartum (dpp), before mutants phenotypically diverge from wild-type (FIG. S1 of Carmell et al., Developmental Cell 12: 503-514, 2007, incorporated by reference). This is precisely what would be expected if nonsense-mediated decay were acting on the predicted mRNA containing numerous premature stop codons. However, all of the coding capacity of Miwi2 still exists in the mutant genome, and splicing around the insertion could conceivably produce a functional Miwi2 transcript. Using RT-PCR primers (that flank the duplicated exons) to amplify wild-type Miwi2 transcripts in testes of 14-day-old animals, we could not detect any wild-type transcript that would be produced by such a splicing event in Miwi2 mutant animals. Thus, we can assert with confidence that our allele produces, at the very least, a severe hypomorph and is likely a null allele.

Mice heterozygous for the Miwi2 mutant allele grew to adulthood, were fertile, and appeared phenotypically normal. Upon intercrossing, it became obvious that male mice homozygous for a mutant allele of Miwi2 were infertile, although they exhibited normal sexual behavior. Homozygous females, however, were fertile and had no obvious defects. Males and females of both sexes were of normal size and weight and had the expected life span.

Initial histological examination (hematoxylin and eosin staining) of testes of adult Miwi2 mutants revealed a very obvious and severe phenotype. Although all other reproductive organs were of normal size and appearance, Miwi2 mutant testes were substantially smaller than their wild-type or heterozygous counterparts. In juveniles at 10 dpp, wild-type and mutant testes were indistinguishable both morphologically (not shown) and histologically. However, cellular defects became apparent a few days later as germ cells proceeded through the first round of spermatogenesis.

Mouse spermatogenesis is a highly regular process that takes about 35 days to complete (de Rooij and Grootegoed, 1998). Spermatogonia, a very small percentage of which are stem cells, line the periphery of the seminiferous tubule and divide mitotically to maintain the stem cell population throughout the lifetime of the animal. These divisions also give rise to differentiating cells that undergo several rounds of mitotic division before entering meiosis. Meiotic cells, or spermatocytes, advance through meiotic prophase I, which can be separated into five phases. In leptotene (phase 1), duplicated chromosomes begin to condense. More extensive pairing and the formation of synaptonemal complexes occur in zygotene (phase 2), and are completed in pachytene (phase 3), when crossing over occurs. Homologs begin to separate in diplotene (phase 4), and chromosomes move apart in diakinesis (phase 5). Prophase I is followed by two meiotic divisions that eventually generate haploid products. The immediate product of meiosis is the round spermatid, which will mature and elongate until being released into the lumen of the tubule.

At the stage when tubules of wild-type siblings contained germ cells at the zygotene and pachytene phases of meiosis I, germ cells in the mutant became noticeably atypical. Two abnormal nuclear morphologies were observed in mutant spermatocytes. In about 80% of abnormal spermatocytes, the nuclei were very condensed and stained intensely with hematoxylin and DAPI. The remaining 20% of abnormal nuclei were extremely large and had an “exploded” morphology with apparently scattered chromatin. The two types of abnormal nuclei appear simultaneously. Therefore, it is unlikely that the same cell transitions from one nuclear morphology to the other. Mutant spermatocytes never proceeded further into, or completed, meiosis I. Consequently, histological examination also revealed that mutant testes contained no postmeiotic cell types such as haploid spermatids or mature sperm. Instead, mutant testes degenerated with age.

To examine the apparent meiotic defect more closely, we tracked the progress of synapsis by using spermatocyte spreads. When spreads were prepared from mutant testes, the vast majority of spermatocytes (>95%) were in the leptotene stage, with about 3% in the zygotene stage and almost nothing in the pachytene stage (in contrast, the heterozygous animal has 22% lepotene, 35% zygotene, and 43% pachytene). At this stage, Scp3, a component of the axial element of the synaptonemal complex, becomes associated with the two sister chromatids of each homolog (Lammers et al., 1994; Moens et al., 1987). Only a few percent of mutant spermatocytes reached zygotene, when longer paired and unpaired axial elements are observed. Normal pachytene spermatocytes with fully condensed, paired chromosomes were never observed in mutant animals. These results showed that mutant spermatocytes arrest before the pachytene stage of meiosis I.

Phosphorylated histone H2AX (g-H2AX) marks the sites of Spo11-induced DNA double-strand breaks that occur during leptotene (Celeste et al., 2002; Fernandez-Capetillo et al., 2003; Hamer et al., 2003; Mahadevaiah et al., 2001). In wild-type cells, double-strand breaks were repaired normally, and most of the g-H2AX signal disappeared as cells entered pachytene. In Miwi2 mutant spermatocytes, g-H2AX staining appeared normal during the leptotene stage. However, concomitant with the change in morphology to highly condensed nuclei, mutant spermatocytes appeared to stain more intensely for g-H2AX as compared to wild-type zygotene cells. The persistence and strength of the g-H2AX staining may indicate the presence of unrepaired double-strand breaks and/or widespread asynapsis, as the cells failed to progress successfully to pachytene. Similar patterns have been observed previously, as mutants defective in synapsis or double-strand break repair fail to eliminate g-H2AX from bulk chromatin (Barchi et al., 2005; Wang and Hoog, 2006; Xu et al., 2003).

During male meiotic prophase, the incorporation of the X and Y chromosomes into the sex or XY body correlates with their transcriptional silencing. By pachytene stage, a second wave of g-H2AX accumulates in the sex body in association with the unsynapsed axial cores of the sex chromosomes (de Vries et al., 2005; Turner et al., 2005). When using standard histological staining, the “exploded” nuclei in Miwi2 mutants often contained structures that look remarkably like sex bodies (Solari, 1974); however, these fail to stain with g-H2AX despite its appearance on the scattered chromatin. At this time, it is unknown whether these structures contain the sex chromosomes or whether other proteins known to populate the sex body are present. This structure may also be a nuclear organelle, such as the nucleolus, that is not normally as prominent at this stage. Nevertheless, we consistently fail to observe a g-H2AX focus in Miwi2 mutants that is characteristic of a successfully formed sex body.

As Miwi2 mutant animals aged, they exhibited dramatically increased levels of apoptosis in the seminiferous tubules as compared to wild-type. A fluorescent TUNEL assay revealed that, while a section through a wild-type testis showed few or no apoptotic cells, a large fraction of tubules in the mutant had many dying cells. These developmental abnormalities arose during prophase of meiosis I. Although occasional TUNEL-positive spermatocytes were present in many tubule sections, larger groups of apoptotic spermatocytes were found in epithelial stage IV, characterized by the presence of mitotic intermediate spermatogonia and early B spermatogonia. The apoptosis of spermatocytes in stage IV resulted in the absence of spermatocytes in later stages, except for a few that entered apoptosis a little more slowly and disappeared in stages V-VII. While the apoptosis of virtually all spermatocytes in stage IV has been observed in many mutants defective in meiotic genes (Barchi et al., 2005; de Rooij and de Boer, 2003), the Miwi2 mutation elicits a unique spermatocyte behavior, as they either condense or enlarge long before they reach epithelial stage IV and apoptose.

In light of these results, we concluded that the seemingly more intense g-H2AX staining of mutant spermatocytes was not due to the creation of double-strand breaks upon induction of apoptosis, as the observed tubules had not yet reached stage IV.

As mutant animals aged, their seminiferous tubules became increasingly vacuolar. Staining with germ cell nuclear antigen (GCNA), which is expressed in all germ cells, indicated that Miwi2 mutants exhibited a marked decrease in the number of germ cells with age. Before the onset of meiosis, the number of germ cells was indistinguishable from that in wild-type. However, with age, mutant tubules contained fewer spermatogonia and abnormal spermatocytes. Tubules lacking germ cells and containing only Sertoli cells began appearing as early as 3 months of age. As the animals aged, Sertoli-cell-only tubules increased in number and became predominant. The Sertoli cells that populate these germ cell-less tubules appeared histologically normal.

Spermatogenic failure and germ cell loss can result from defects in germ cells or in their somatic environment (Brinster, 2002). In addition to being expressed in premeiotic germ cells, Miwi2 is expressed at significant levels in c-kit mutant testes (W/Wv) that are virtually germ cell free (Silvers, 1979) and is also detectable in the TM4 Sertoli cell line (FIG. S1 or Carmell et al., Developmental Cell 12: 503-514, 2007, incorporated by reference). Thus, we sought to determine whether the defects observed in Miwi2 mutant testes reflect a cell-autonomous defect in the germ cells themselves or whether MIWI2 plays a critical role in somatic support cells.

To address this question, we transplanted wild-type germ cells into Miwi2 mutant testes to assess the integrity of the mutant soma. Recipient animals reconstituted complete spermatogenesis in a subset of tubules, with successful completion of both meiotic divisions and production of mature sperm. These spermatogenic tubules existed side by side with noncolonized tubules that displayed the characteristic Miwi2 mutant phenotype. Although our conclusions must be tempered by the remote possibility that the mutant soma could harbor a level of Miwi2 that escapes detection by RT-PCR, these studies strongly suggest that Miwi2 mutant soma can successfully support germ cells and lead to the conclusion that wild-type levels of Miwi2 expression in the germ cells themselves is necessary and sufficient to support meiosis and spermiogenesis.

Two lines of circumstantial evidence point to a potential role for mammalian Piwi proteins in transposon control. First, in Drosophila, Piwi proteins have a demonstrated role in the control of transposons (Aravin et al., 2001, 2004; Kalmykova et al., 2005; Saito et al., 2006; Sarot et al., 2004; Savitsky et al., 2006; Vagin et al., 2004, 2006). Transposon activation results in both germline and embryonic defects that result in female sterility through a phenomenon called hybrid dysgenesis. This is characterized by a depletion of germline stem cells, abnormal oogenesis, and defects in oocyte organization. Second, a link between the inappropriate expression of certain repetitive elements and meiotic arrest has previously been demonstrated in mammals. In particular, animals bearing mutations in a catalytically defective member of the DNA methyltransferase family, DNMT3L, fail to methylate transposons in the male germline, resulting in abnormal and abundant expression from several transposon families (Bourc'his and Bestor, 2004; Hata et al., 2006; Webster et al., 2005). This phenomenon is correlated with a meiotic arrest prior to pachytene as well as germ cell loss. We therefore considered that the germ cell loss and prevalent apoptosis that we observe in Miwi2 mutants might correlate with transposon activation.

To investigate whether Miwi2 mutation affected expression from normally silent transposons, we used in situ hybridization of testes of the various genotypes of animals, with probes recognizing the sense strands of LINE-1 and IAP elements. When using this method, long interspersed elements (LINEs) are not detectable in adult wild-type testes. However, in Miwi2 mutants, a strong signal can be seen with probes that detect sense-oriented LINE-1 transcripts. Similar approaches were also used to monitor expression of intracisternal A particle (IAP) elements that belong to the most active class of LTR retrotransposons in the mouse. Sense strand IAP transcripts were undetectable by in situ hybridization in wildtype animals, while they were readily detectible in Miwi2 mutants.

We also used quantitative RT-PCR analysis of transposable elements in 14-day-old animals. Elevated levels of transcripts were detected exclusively in germ lineages, with no apparent activation in Sertoli or interstitial cells of the testes. Results from in situ analyses were supported and extended by such quantitative RT-PCR results. A 7- to 12-fold increase in LINE-1 expression was detected in the mutants relative to heterozygous animals when primers directed to the 5′UTR and ORF2 were used. Similar results were obtained with strand-specific RT-PCR measuring only sense-orientation LINE-1 transcripts (not shown). IAP elements were activated more modestly. Elevated expression of these elements was detected only in the testes, and not in the kidneys, of mutant animals (data not shown).

To ensure that the observed effects were not a secondary consequence of meiotic arrest, we analyzed testes from meiosis defective-1 (Mei1) mutant animals, which display a meiotic arrest phenotype similar to Miwi2 mutants, and failed to observe increased transposon expression.

Transposable elements are thought to be maintained in a silent state by DNA methylation and packaging into heterochromatin. We investigated the methyation status of LINE-1 in the Miwi2 mutants by Southern blot analysis after digestion with a methylation-sensitive enzyme, HpaII. Specifically, DNA isolated from the tail or testes of wildtype, heterozygous, and Miwi2 mutant animals was digested with either methylation-insensitive (MspI, M) or methylation-sensitive (HpaII, H) restriction enzymes. Southern blot analysis of these DNAs was conducted, and membranes were probed with a fragment of the LINE-1 5′UTR. The probe recognizes four bands of 156 bp generated by HpaII sites in the 5′UTR, and a band of 1206 bp that is generated by one HpaII site in the 5′UTR and one site in the coding sequence.

We found that LINE-1 elements become demethylated in Miwi2 mutants as compared to wild-type and heterozygous animals. Demethylation was detected specifically in DNA prepared from the testes and not from the tail. Thus, compromising Miwi2 can affect the methylation of repetitive elements specifically in the germline. For comparison, we assayed LINE-1 methylation in testes from several mutants that show a meiotic arrest similar to Miwi2 mutants (FIG. S2 of Carmell et al., Developmental Cell 12: 503-514, 2007, incorporated by reference). None of these mutant animals show LINE-1 demethylation.

We then used bisulfite sequencing to examine methylation of the first 150 bp of the 5′UTR of a specific copy of L1Md-A2. Lollipop representation was used to depict the sequences obtained after bisulfite treatment of Miwi2^{+/-} and −/− testis DNA. The first 150 bp of a specific L1 element were selectively amplified and analyzed for the presence of methylated CpGs. Methylated and unmethylated CpGs are represented as filled and empty lollipops, respectively. Out of 75 sequences obtained for each genotype, 20 randomly chosen sequences are shown. Information on the complete set can be found in FIG. S3 of Carmell et al. (Developmental Cell 12: 503-514, 2007, incorporated by reference).

In heterozygous animals, this region is almost completely methylated, with 95% of all CpGs modified. In the mutant, only 60% of CpGs are methylated overall, with two distinct populations of PCR products being apparent. These are represented at the extremes by 34% of the clones that are completely unmethylated, and 46% that retain full methylation (FIG. S3 of Carmell et al., Developmental Cell 12: 503-514, 2007, incorporated by reference). Based on our Southern blot and quantitative RT-PCR analyses that show normal methylation and transposon repression in somatic tissues, we suggest that these two populations are likely derived from germ cells (unmethylated) and somatic cells (methylated).

Combined, these results show that Miwi2 mutants derepress and demethylate transposable elements.

Successful expansion by selfish genetic elements can only occur if increased copy numbers can be transmitted to the next generation. Consistent with this notion, LINE and IAP elements are known to be active almost exclusively in the germline (Branciforte and Martin, 1994; Dupressoir and Heidmann, 1996). Full-length sense strand LINE-1 transcripts, and the ORF1 protein that they encode, have been detected in leptotene and zygotene spermatocytes in pubertal mouse testes (Branciforte and Martin, 1994). In the adult male, truncated transcripts and ORF1 protein are present in somatic cells and haploid germ cells (Branciforte and Martin, 1994; Trelogan and Martin, 1995). ORF1 protein is also present in oocytes and steroidogenic cells in the female germline (Branciforte and Martin, 1994; Trelogan and Martin, 1995). Considering the deleterious and cumulative effects of unregulated repetitive element expansion, there should be tremendous evolutionary pressure to evolve effective transposon control strategies in the germline. Our data indicate that mammalian Piwi proteins form at least part of such a defense mechanism.

In Drosophila, Piwi proteins are reported to have both cell autonomous and nonautonomous roles in maintaining the integrity of the germline (Cox et al., 2000). In particular, piwi mutants lose germ cells as a result of functions for this protein in the germ cells themselves and in maintaining the integrity of the germline stem cell niche. In mammals, Miwi and Mili mutants arrest spermatogenesis at different stages, but neither is reported to lose germ cells, as might be expected if, like PIWI, either protein had a role in stem cell maintenance. Here, we show that disruption of Miwi2 creates two distinct phenotypes in the male germline of mice. First, Miwi2 mutant germ cells that enter prophase of meiosis I arrest prior to the pachytene stage. Second, Miwi2 mutants progressively lose germ cells and accumulate tubules that contain only somatic Sertoli cells. The latter observation suggests that MIWI2 may conserve some of the stem cell maintenance functions played by PIWI in Drosophila. It is presently unclear whether the requirement for Piwi proteins in stem cell maintenance in flies is due to their role in regulating gene expression, or whether the phenotypes of Piwi-family mutations can be solely explained by loss of transposon control.

Accumulating data have suggested that Drosophila Piwi proteins play a prominent and essential role in transposon control (Aravin et al., 2001, 2004; Kalmykova et al., 2005; Sarot et al., 2004; Savitsky et al., 2006; Vagin et al., 2004). One consequence of disrupting transposon suppression in flies is the appearance of DNA damage, as evidenced by the accumulation of phosphorylated histone H2AX (Belgnaoui et al., 2006; Gasior et al., 2006). A key role for DNA-damage pathways in the ultimate output of Piwi family mutations, production of defective oocytes, is indicated by the fact that mutation of key DNA-damage sensing pathways can at least partially suppress the effects of transposon activation (Klattenhoff et al., 2007). Our results point to a previously unsuspected role for mammalian Piwi proteins in the control of transposons in the male germline.

As in flies, Miwi2 mutations also result in accumulation of DNA damage, as indicated by g-H2AX accumulation. The relationship between the molecular phenotypes of Piwi family mutations in flies and mice, particularly whether activation of DNA-damage response pathways plays a role in the meiotic defects observed in Miwi2 mutants, remains to be determined.

Drosophila Piwi proteins interact with small RNAs of about 24-26 nucleotides in length (Aravin et al., 2001; Saito et al., 2006; Vagin et al., 2006). These are highly enriched for sequences that target repetitive elements and are therefore called rasiRNAs (repeat-associated siRNAs) (Aravin et al., 2003; Saito et al., 2006). In contrast, mammalian Piwi-family proteins, MIWI and MILI, bind to an about 26-30 nucleotide class of small RNAs known as piRNAs (Piwi-interacting RNAs) (Aravin et al., 2006; Girard et al., 2006; Grivna et al., 2006; Lau et al., 2006; Watanabe et al., 2006). A large proportion of piRNAs are only complimentary to the loci from which they came, leading to the hypothesis that the piRNA loci themselves must be the targets of MILI and MIWI RNPs. Results presented here point to a role for piRNAs in transposon control in mammals similar to those that have been demonstrated for rasiRNAs in Drosophila.

Unexpectedly, we have found that the rasiRNA system in flies shows many characteristics in common with the piRNA system in mammals (Brennecke et al., 2007). Piwi-interacting RNAs in Drosophila are derived from discrete genomic loci. At least some of these loci show the profound strand asymmetry that characterizes mammalian piRNA loci. These observations begin to unify Piwi protein functions in disparate organisms. However, future work will be required to understand how the meiotic piRNA loci, which are depleted of repeats, relate functionally to the piRNA loci in flies that act as master controllers of transposon activity.

Silencing of mammalian transposons depends on their methylation status (Bourc'his and Bestor, 2004). Genomes of primordial germ cells undergo demethylation followed by de novo remethylation in prospermatogonia, a nondividing cell type that exists only in the perinatal period. How the patterns of methylation are determined in developing germ cells is not understood. In Arabidopsis, it is well established that the RNAi machinery can use small RNAs to direct genomic methylation, though the precise biochemical mechanism underlying these events remains unclear (Matzke and Birchler, 2005). In plants, ARGONAUTE4, a member of the Argonaute rather than the Piwi subfamily, binds to 24 nt, small RNAs and mainly directs asymmetric cytosine methylation (CpNpG and CpHpH). However, such asymmetric methylation is rare or absent in mammalian genomes. Here, we provide evidence that loss of MIWI2 function affects the methylation status of LINE-1 elements. MIWI2 complexes, which we presume are directed to their targets by associated piRNAs, might help to establish genomic methylation patterns on repetitive elements during germ cell development. It is also possible that removal of MIWI2 interferes with the maintenance of genomic methylation patterns that normally occurs in dividing spermatagonia. A detailed analysis of patterns of Miwi2 expression and identification of piRNAs that interact with MIW12 during germ cell development will be needed to distinguish roles for this protein complex in de novo versus maintenance methylation.

### EXPERIMENTAL PROCEDURES

Gene Targeting and Mice

The Miwi2 targeting construct was obtained by screening of the lambda phage 30 HPRT library described by Zheng et al. (1999) that is now the basis of the MICER system (Adams et al., 2004). The resultant targeting construct, containing exons 9-12 of Miwi2, was electroporated into AB2.2 mouse embryonic stem (ES) cells. Targeted clones were injected into C57BL/6 blastocysts to generate eight high percentage chimeras, four of which were able to pass the allele through the germline. Results presented herein were obtained from mice with a mixed 129/B6 background. In general, younger animals were back-crossed to B6 4-6 generations, and older animals were back-crossed less. Mouse genotyping was performed by Southern blot analysis after digestion of genomic DNA with AccI. The 332 bp probe was amplified from genomic DNA with primers described in Table S1.

Histology

Testes were collected and fixed in Bouin's fixative at 4° C. overnight, then dehydrated to 70% ethanol. After embedding in paraffin, 8 mm sections were made by using a microtome. For routine histology, sections were stained with hematoxylin and eosin. For routine histology and subsequent staining, at least three animals of each age and genotype were examined.

Immunohistochemistry

Slides were rehydrated and treated with 3% hydrogen peroxide for 10 min. Blocking was carried out in 5% goat serum, 1% BSA in PBS for 10 min. Slides were incubated overnight at 4° C. with primary antibody as follows. Antibody to g-H2AX (Upstate) was used at 1:150 in 1% BSA in PBS. GCNA (a gift of G. Enders) was used neat. Detection was performed by using the Vector ABC kit according to the manufacturer's directions, except 2 ml each of solutions A and B were used per milliliter of PBS. Slides were counterstained with Mayer hematoxylin, mounted with Histomount mounting media, and coverslipped.

For immunocytological analysis of synaptonemal complex formation, surface spreading of spermatocytes was performed as described by Matsuda et al. (1992). Spreads were hybridized with goat anti-Scp3 (gift of T. Ashley) at 1:400 dilution. Approximately 200 nuclei from each of three animals were counted, for a total of 600 nuclei of each genotype. Spreads were conducted on animals at 16 dpp.

TUNEL Assay

Slides containing Bouin's-fixed testes sections were rehydrated and microwaved for 5 min in 10 mMCitrate buffer (pH 6.0). After incubation in 3% hydrogen peroxide, slides were incubated with 0.3 U/microliter deoxynucleotidal terminal transferase (Amersham) and 6.66 mMbiotin-16-dUTP (Roche) for 1 hr at 37° C. After washing in 300 mM NaCl, 30 mM NaCitrate in MilliQ water for 15 min at room temperature, slides were blocked in 2% BSA in PBS for 10 min. Slides were incubated in a 1:20 dilution of ExtrAvidine peroxidase (Sigma) in 1% BSA in PBS for 30 min at 37° C. Detection was achieved by using diaminobenzidine.

Slides were counterstained with Mayer hematoxylin, dehydrated, and mounted. Fluorescent TUNEL assay was conducted by using the Roche In Situ Cell Death Detection kit according to the manufacturer's instructions.

Germ Cell Transplants

Transplants were carried out as described by Buaas et al. (2004). Donor cells were harvested from the transgenic mouse line C57BL/6.129-TgR(Rosa26)26S (Jackson Laboratory). Donor cells were transplanted into testes of Miwi2 mutant mice that were already somewhat germ cell depleted due to the mutation, or into W/Wv mice that have no endogenous spermatogenesis as a control (Jackson Laboratory, WBB6F1/Jkit W/KitWv). Recipient testes were analyzed with standard histological methods to identify areas of colonization by donor cells. One out of 10 Miwi2 mutant recipients and 2 out of 5 W/Wv were successfully colonized.

RT-PCR and QPCR

Total RNA was extracted from mouse tissues by using Trizol according to the manufacturer's recommendations. cDNA was synthesized by using Superscript III Reverse Transcriptase (Invitrogen) on RNA primed with random hexamers. QPCR was carried out by using Sybr Green PCR Master Mix (Applied Biosystems) on a Biorad Chromo 4 Real Time system. Two animals of each genotype were examined, with the exception of Mei1, for which we had only one specimen. Assays were done in triplicate. Miwi2 animals were 14 days old, and Mei1 animals were 21 days old. Primers Miwi2-F and Miwi2-R are downstream of the duplicated exons and cannot distinguish between wild-type and mutant transcript. Primers Miwi2-exon7F and Miwi2-exon14R flank the duplicated exons in the mutant transcript and therefore assay for only the wild-type transcript. The wild-type transcript produces a band of 1006 bp, while the mutant would yield a larger product due to the duplication of exons 9-12. Primers are listed in Table S1.

In Situ Hybridization

In situ hybridization was done as described by Bourc'his and Bestor (2004). The 50LTR IAP probe was as described by Walsh et al. (1998), and the LINE-1 50UTR probe is complementary to a type A LINE-1 element (GenBank accession number: M13002, nucleotides 515-1,628) (Bourc'his and Bestor, 2004).

Methylation Southern Blot Analysis

Southern blot analysis to assay for methylation was done as described by Bourc'his and Bestor (2004). The same LINE-1 50UTR probe was used as for in situ hybridization, except a gel-purified fragment was random prime labeled by using the Rediprime II kit (Amersham). DNA from testis and tail were digested with the methylation-sensitive enzyme HpaII and its methylation-insensitive isoschizomer, MspI.

Bisulfite DNA Sequencing

DNA from Miwi2^{+/-} and −/− testes was bisulfite treated and purified by using the EZ DNA Methylation Gold kit (Zymo Research). Primers MethylL1-F and MethylL1-R were designed to specifically amplify one occurrence of L1 Md-A2 located on chromosome X. The PCR products were then gel purified, TOPO cloned (Invitrogen), sequenced, and analyzed by using BiQ-Analyzer (Bock et al., 2005). Primers and the sequence of the amplified region are given in Table S1.

Supplemental Data

Supplemental Data include analysis of Miwi2 expression, transposon demethylation controls, the entire bisulfite DNA-sequencing data set, and primer sequences and are available at http://www.developmentalcell.com/cgi/content/full/12/4/503/DC1/.

### REFERENCES CITED FOR EXAMPLE VII

Adams, D. J., Biggs, P. J., Cox, T., Davies, R., van der Weyden, L., Jonkers, J., Smith, J., Plumb, B., Taylor, R., Nishijima, I., et al. (2004). Mutagenic insertion and chromosome engineering resource (MICER). Nat. Genet. 36, 867-871.
Aravin, A., Gaidatzis, D., Pfeffer, S., Lagos-Quintana, M., Landgraf, P., lovino, N., Morris, P., Brownstein, M. J., Kuramochi-Miyagawa, S., Nakano, T., et al. (2006). A novel class of small RNAs bind to MILI protein in mouse testes. Nature 442, 203-207.
Aravin, A. A., Naumova, N. M., Tulin, A. V., Vagin, V. V., Rozovsky, Y. M., and Gvozdev, V. A. (2001). Double-stranded RNA-mediated silencing of genomic tandem repeats and transposable elements in the D. melanogaster germline. Curr. Biol. 11, 1017-1027.
Aravin, A. A., Lagos-Quintana, M., Yalcin, A., Zavolan, M., Marks, D., Snyder, B., Gaasterland, T., Meyer, J., and Tuschl, T. (2003). The small RNA profile during Drosophila melanogaster development. Dev. Cell 5, 337-350.
Aravin, A. A., Klenov, M. S., Vagin, V. V., Bantignies, F., Cavalli, G., and Gvozdev, V. A. (2004). Dissection of a natural RNA silencing process in the Drosophila melanogaster germ line. Mol. Cell. Biol. 24, 6742-6750.
Barchi, M., Mahadevaiah, S., Di Giacomo, M., Baudat, F., de Rooij, D. G., Burgoyne, P. S., Jasin, M., and Keeney, S. (2005). Surveillance of different recombination defects in mouse spermatocytes yields distinct responses despite elimination at an identical developmental stage. Mol. Cell. Biol. 25, 7203-7215.
Belgnaoui, S. M., Gosden, R. G., Semmes, O. J., and Haoudi, A. (2006). Human LINE-1 retrotransposon induces DNA damage and apoptosis in cancer cells. Cancer Cell Int. 6, 13.
Bock, C., Reither, S., Mikeska, T., Paulsen, M., Walter, J., and Lengauer, T. (2005). BiQ Analyzer: visualization and quality control for DNA methylation data from bisulfite sequencing. Bioliformatics 21, 4067-4068.
Bourc'his, D., and Bestor, T. H. (2004). Meiotic catastrophe and retrotransposon reactivation in male germ cells lacking Dnmt3L. Nature 431, 96-99.
Branciforte, D., and Martin, S. L. (1994). Developmental and cell type specificity of LINE-1 expression in mouse testis: implications for transposition. Mol. Cell. Biol. 14, 2584-2592.
Brennecke, J., Aravin, A. A., Stark, A., Dus, M., Kellis, M., Sachidanandam, R., and Hannon, G. J. (2007). Discrete small RNA-generating loci as master regulators of transposon activity in Drosophila. Cell, in press. Published online Mar. 8, 2007. 10.1016/j.cell.2007.01.043.
Brinster, R. L. (2002). Germline stem cell transplantation and transgenesis. Science 296, 2174-2176.
Buaas, F. W., Kirsh, A. L., Sharma, M., McLean, D. J., Morris, J. L., Griswold, M. D., de Rooij, D. G., and Braun, R. E. (2004). Plzf is required in adult male germ cells for stem cell self-renewal. Nat. Genet. 36, 647-652.
Celeste, A., Petersen, S., Romanienko, P. J., Femandez-Capetillo, O., Chen, H. T., Sedelnikova, O. A., Reina-San-Martin, B., Coppola, V., Meffre, E., Difilippantonio, M. J., et al. (2002). Genomic instability in mice lacking histone H2AX. Science 296, 922-927.
Cox, D. N., Chao, A., Baker, J., Chang, L., Qiao, D., and Lin, H. (1998). A novel class of evolutionarily conserved genes defined by piwi are essential for stem cell self-renewal. Genes Dev. 12, 3715-3727.
Cox, D. N., Chao, A., and Lin, H. (2000). piwi encodes a nucleoplasmic factor whose activity modulates the number and division rate of germline stem cells. Development 127, 503-514.
de Rooij, D. G., and de Boer, P. (2003). Specific arrests of spermatogenesis in genetically modified and mutant mice. Cytogenet. Genome Res. 103, 267-276.
de Rooij, D. G., and Grootegoed, J. A. (1998). Spermatogonial stem cells. Curr. Opin. Cell Biol. 10, 694-701.
de Vries, F. A., de Boer, E., van den Bosch, M., Baarends, W. M., Ooms, M., Yuan, L., Liu, J. G., van Zeeland, A. A., Heyting, C., and Pastink, A. (2005). Mouse Sycp1 functions in synaptonemal complex assembly, melotic recombination, and XY body formation. Genes Dev. 19, 1376-1389.
Deng, W., and Lin, H. (2002). miwi, a murine homolog of piwi, encodes a cytoplasmic protein essential for spermatogenesis. Dev. Cell 2, 819-830.
Dupressoir, A., and Heidmann, T. (1996). Germ line-specific expression of intracisternal A-particle retrotransposons in transgenic mice. Mol. Cell. Biol. 16, 4495-4503.
Fernandez-Capetillo, O., Mahadevaiah, S. K., Celeste, A., Romanienko, P. J., Camerini-Otero, R. D., Bonner, W. M., Manova, K., Burgoyne, P., and Nussenzweig, A. (2003). H2AX is required for chromatin remodeling and inactivation of sex chromosomes in male mouse meiosis. Dev. Cell 4, 497-508.
Gasior, S. L., Wakeman, T. P., Xu, B., and Deininger, P. L. (2006). The human LINE-1 retrotransposon creates DNA double-strand breaks. J. Mol. Biol. 357, 1383-1393.
Girard, A., Sachidanandam, R., Hannon, G. J., and Carmell, M. A. (2006). A germline-specific class of small RNAs binds mammalian Piwi proteins. Nature 442, 199-202.
Grivna, S. T., Beyret, E., Wang, Z., and Lin, H. (2006). A novel class of small RNAs in mouse spermatogenic cells. Genes Dev. 20, 1709-1714.
Hamer, G., Roepers-Gajadien, H. L., van Duyn-Goedhart, A., Gademan, I. S., Kal, H. B., van Buul, P. P., and de Rooij, D. G. (2003). DNA double-strand breaks and g-H2AX signaling in the testis. Biol. Reprod. 68, 628-634.
Hata, K., Kusumi, M., Yokomine, T., Li, E., and Sasaki, H. (2006). Meiotic and epigenetic aberrations in Dnmt3L-deficient male germ cells. Mol. Reprod. Dev. 73, 116-122.
Kalmykova, A. I., Klenov, M. S., and Gvozdev, V. A. (2005). Argonaute protein PIWI controls mobilization of retrotransposons in the Drosophila male germline. Nucleic Acids Res. 33, 2052-2059.
Klattenhoff, C., Bratu, D. P., McGinnis-Schultz, N., Koppetsch, B. S., Cook, H. A., and Theurkauf, W. E. (2007). Drosophila rasiRNA pathway mutations disrupt embryonic axis specification through activation of an ATR/Chk2 DNA damage response. Dev. Cell 12, 45-55.
Kuramochi-Miyagawa, S., Kimura, T., Yomogida, K., Kuroiwa, A., Tadokoro, Y., Fujita, Y., Sato, M., Matsuda, Y., and Nakano, T. (2001). Two mouse piwi-related genes: miwi and mili. Mech. Dev. 108, 121-133.
Kuramochi-Miyagawa, S., Kimura, T., Ijiri, T. W., Isobe, T., Asada, N., Fujita, Y., Ikawa, M., Iwal, N., Okabe, M., Deng, W., et al. (2004). Mili, a mammalian member of piwi family gene, is essential for spermatogenesis. Development 131, 839-849.
Lammers, J. H., Offenberg, H. H., van Aalderen, M., Vink, A. C., Dietrich, A. J., and Heyting, C. (1994). The gene encoding a major component of the lateral elements of synaptonemal complexes of the rat is related to X-linked lylphocyte-regulated genes. Mol. Cell. Biol. 14, 1137-1146.
Lau, N. C., Seto, A. G., Kim, J., Kuramochi-Miyagawa, S., Nakano, T., Bartel, D. P., and Kingston, R. E. (2006). Characterization of the piRNA complex from rat testes. Science 313, 363-367.

Lin, H., and Spradling, A. C. (1997). A novel group of pumilio mutations affects the asymmetric division of germline stem cells in the Drosophila ovary. Development 124, 2463-2476.
Mahadevaiah, S. K., Turner, J. M., Baudat, F., Rogakou, E. P., de Boer, P., Blanco-Rodriguez, J., Jasin, M., Keeney, S., Bonner, W. M., and Burgoyne, P. S. (2001). Recombinational DNA double-strand breaks in mice precede synapsis. Nat. Genet. 27, 271-276.
Matsuda, Y., Moens, P. B., and Chapman, V. M. (1992). Deficiency of X and Y chromosomal pairing at meiotic prophase in spermatocytes of sterile interspecific hybrids between laboratory mice (Mus domesticus) and Mus spretus. Chromosoma 101, 483-492.
Matzke, M. A., and Birchler, J. A. (2005). RNAi-mediated pathways in the nucleus. Nat. Rev. Genet. 6, 24-35.
Moens, P. B., Heyting, C., Dietrich, A. J., van Raamsdonk, W., and Chen, Q. (1987). Synaptonemal complex antigen location and conservation. J. Cell Biol. 105, 93-103.
Saito, K., Nishida, K. M., Mori, T., Kawamura, Y., Miyoshi, K., Nagami, T., Siomi, H., and Siomi, M. C. (2006). Specific association of Piwi with rasiRNAs derived from retrotransposon and heterochromatic regions in the Drosophila genome. Genes Dev. 20, 2214-2222.
Sarot, E., Payen-Groschene, G., Bucheton, A., and Pelisson, A. (2004). Evidence for a piwi-dependent RNA silencing of the gypsy endogenous retrovirus by the Drosophila melanogaster flamenco gene. Genetics 166, 1313-1321.
Sasaki. T., Shiohama, A., Minoshima, S., and Shimizu, N. (2003). Identification of eight members of the Argonaute family in the human genome small star, filled. Genomics 82, 323-330.
Savitsky, M., Kwon, D., Georgiev, P., Kalmykova, A., and Gvozdev, V. (2006). Telomere elongation is under the control of the RNAi-based mechanism in the Drosophila germline. Genes Dev. 20, 345-354.
Schmidt, A., Palumbo, G., Bozzetti, M. P., Tritto, P., Pimpinelli, S., and Schafer, U. (1999). Genetic and molecular characterization of sting, a gene involved in crystal formation and meiotic drive in the male germ line of Drosophila melanogaster. Genetics 151, 749-760.
Silvers, W. K. (1979). The Coat Colors of Mice (New York: Springer Verlag).
Solari, A. J. (1974). The behavior of the XY pair in mammals. Int. Rev. Cytol. 38, 273-317.
Trelogan, S. A., and Martin, S. L. (1995). Tightly regulated, developmentally specific expression of the first open reading frame from LINE-1 during mouse embryogenesis. Proc. Natl. Acad. Sci. USA 92, 1520-1524.
Turner, J. M., Mahadevaiah, S. K., Fernandez-Capetillo, O., Nussenzweig, A., Xu, X., Deng, C. X., and Burgoyne, P. S. (2005). Silencing of unsynapsed meiotic chromosomes in the mouse. Nat. Genet. 37, 41-47.
Vagin, V. V., Klenov, M. S., Kalmykova, A. I., Stolyarenko, A. D., Kotelnikov, R. N., and Gvozdev, V. A. (2004). The RNA interference proteins and vasa locus are involved in the silencing of retrotransposons in the female germline of Drosophila melanogaster. RNA Biol. 1, 54-58.
Vagin, V. V., Sigova, A., Li, C., Seitz, H., Gvozdev, V., and Zamore, P. D. (2006). A distinct small RNA pathway silences selfish genetic elements in the germline. Science 313, 320-324.
Walsh, C. P., Chaillet, J. R., and Bestor, T. H. (1998). Transcription of IAP endogenous retroviruses is constrained by cytosine methylation. Nat. Genet. 20, 116-117.
Wang, H., and Hoog, C. (2006). Structural damage to meiotic chromosomes impairs DNA recombination and checkpoint control in mammalian oocytes. J. Cell Biol. 173, 485-495.
Watanabe, T., Takeda, A., Tsukiyama, T., Mise, K., Okuno, T., Sasaki, H., Minami, N., and Imai, H. (2006). Identification and characterization of two novel classes of small RNAs in the mouse germline: retrotransposon-derived siRNAs in oocytes and germline small RNAs in testes. Genes Dev. 20, 1732-1743.
Webster, K. E., O'Bryan, M. K., Fletcher, S., Crewther, P. E., Aapola, U., Craig, J., Harrison, D. K., Aung, H., Phutikanit, N., Lyle, R., et al. (2005). Meiotic and epigenetic defects in Dnmt3L-knockout mouse spermatogenesis. Proc. Natl. Acad. Sci. USA 102, 4068-4073.
Xu, X., Aprelikova, O., Moens, P., Deng, C. X., and Furth, P. A. (2003). Impaired meiotic DNA-damage repair and lack of crossing-over during spermatogenesis in BRCA1 full-length isoform deficient mice. Development 130, 2001-2012.
Zheng, B., Mills, A. A., and Bradley, A. (1999). A system for rapid generation of coat color-tagged knockouts and defined chromosomal rearrangements in mice. Nucleic Acids Res. 27, 2354-2360.

### EQUIVALENTS

Those skilled in the art will recognize, or be able to ascertain using no more than routine experimentation, many equivalents to the specific embodiments of the invention described herein. Such equivalents are intended to be encompassed by the following claims.

The entire contents of all patents, published patent applications and other references cited herein are hereby expressly incorporated herein in their entireties by reference.

We claim:

1. A method for regulating the expression of a target gene in a cell, comprising introducing into the cell a small single stranded RNA or analog thereof (piRNA) that:
(i) selectively binds to proteins of the Piwi or Aubergine subclasses of Argonaute proteins relative to the Ago3 subclass of Argonaute proteins,
(ii) forms an RNP complex (piRC) with the Piwi or Aubergine proteins, and,
(iii) induces transcriptional and/or post-transcriptional gene silencing,
wherein the piRNA induces transcriptional and/or post-transcriptional gene silencing of the target gene.

2. The method of claim 1, wherein the piRNA is about 25-50 nucleotides in length, about 25-39 nucleotides in length, or about 26-31 nucleotides in length.

3. The method of claim 1, wherein the piRNA preferentially associates with the MILI protein and is about 26-28 nucleotides in length.

4. The method of claim 1, wherein the piRNA comprises a nucleotide sequence that hybridizes under physiologic conditions of a cell to the nucleotide sequence of at least a portion of a genomic sequence of the cell to cause down-regulation of transcription at the genomic level, or to cause down-regulation of transcription of an mRNA transcript for a target gene.

5. The method of claim 4, wherein the piRNA comprises no more than 1 in 5 basepairs of nucleotide mismatches with respect to the target gene mRNA transcript.

6. The method of claim 4, wherein the piRNA is greater than 90% identical to the portion of the target gene mRNA transcript to which it hybridizes.

7. The method of claim 1, wherein the piRNA comprises one or more modifications on phosphate-sugar backbone or on nucleosides.

8. The method of claim 1, wherein the modifications on phosphate-sugar backbone comprise phosphorothioate, phosphoramidate, phosphodithioates, or chimeric methylphosphonate-phosphodiester linkages.

9. The method of claim 1, wherein the modifications on nucleosides comprise 2′-methoxyethoxy, 2′-methyl-thio-ethyl, 2′-deoxy-2′-fluoro, 2′-deoxy-2′-chloro, 2-azido, 2′-O-trifluoromethyl, 2′-O-ethyl-trifluoromethoxy, 2′-O-difluoromethoxy-ethoxy, 4′-thio, or 2′-O-methyl modifications.

10. The method of claim 1, wherein the piRNA comprises a terminal cap moiety at the 5′-end, the 3′-end, or both the 5′ and 3′ ends.

11. The method of claim 1, wherein the piRNA comprises a 5′-U residue.

12. The method of claim 1, wherein the target gene is an insect-specific gene.

13. The method of claim 1, wherein the cell is a stem cell.

14. The method of claim 1, wherein the cell is an embryonic stem cell.

15. The method of claim 1, wherein the cell is in culture.

16. The method of claim 1, wherein the target gene is required or essential for cell growth and/or development, for mRNA degradation, for translational repression, or for transcriptional gene silencing (TGS).

17. A composition or therapeutic formulation comprising the piRNA of claim 1, pharmaceutically acceptable salts, esters or salts of such esters, or bioequivalent compounds thereof, admixed, encapsulated, conjugated or otherwise associated with liposomes, polymers, receptor targeted molecules, oral, rectal, topical or other formulations that assist uptake, distribution and/or absorption.

18. The composition or therapeutic formulation of claim 17, further comprising penetration enhancers, carrier compounds, and/or transfection agents.

19. A polynucleotide comprising two or more concatenated piRNAs, each of said piRNAs comprise a small single stranded RNA or analog thereof that:
(i) selectively binds to proteins of the Piwi or Aubergine subclasses of Argonaute proteins relative to the Ago3 subclass of Argonaute proteins,
(ii) forms an RNP complex (piRC) with the Piwi or Aubergine proteins, and,
(iii) induces transcriptional and/or post-transcriptional gene silencing.

20. The polynucleotide of claim 19, wherein the piRNAs are of the same or different sequences.

21. A polynucleotide encoding one or more piRNA(s) of claim 1, or precursor(s) thereof, wherein said piRNA(s) are transcribed from said polynucleotide, or wherein said precursor(s), when transcribed from said polynucleotide, are metabolized by a cell comprising the polynucleotide to give rise to the piRNA(s) of claim 1.

22. A probe comprising a polynucleotide that hybridizes to the piRNA of claim 1.

23. The probe of claim 22, wherein the polynucleotide is an RNA.

24. The probe of claim 22, comprising at least about 8-22 contiguous nucleotides complementary to the piRNA of claim 1.

25. A plurality of probes of claim 22, for detecting two or more piRNA sequences in a sample.

26. A composition comprising the probe of claim 22, or the plurality of probes of claim 25.

27. A method of detecting the presence or absence of one or more particular piRNA sequences in a sample from the genome of a patient or subject, comprising contacting the sample with the probe of claim 22, or the plurality of probes of claim 25.

28. The method of claim 27, wherein the sample is a cell or a gamete of the patient or subject.

29. A biochip comprising a solid substrate, said substrate comprising a plurality of probes for detecting the piRNA of claim 1.

30. The biochip of claim 29, wherein each of the probes is attached to the substrate at a spatially defined address.

31. The biochip of claim 29, wherein the biochip comprises probes that are complementary to a variety of different piRNA sequences.

32. The biochip of claim 31, wherein the variety of different piRNA sequences are differentially expressed in normal versus disease tissue, or at different stages of development.

33. A method of detecting differential expression of disease-associated piRNA(s), comprising:
(1) contacting a disease sample with a plurality of probes for detecting piRNA sequences,
(2) contacting a control sample with the plurality of probes, and,
(3) identifying one or more of piRNA sequences that are differentially expressed in the disease sample as compared to the control sample,
thereby detecting differential expression of disease-associated piRNA(s).

34. A method of identifying a compound that modulates a pathological condition or a cell/tissue development pathway, the method comprising:
(1) providing a cell that expresses one or more piRNAs as markers for a particular cell phenotype or cell fate of the pathological condition or the cell/tissue development pathway;
(2) contacting the cell with a candidate agent; and,
(3) measuring the expression level of at least one said piRNAs,
wherein a change in the expression level of at least one said piRNAs indicates that the candidate agent is a modulator of the pathological condition or the cell/tissue development pathway.


SEQUENCE LISTING (9 sequences)

SEQ ID NO: 1 - DNA, 19 bp, Artificial Sequence
  Description of Artificial Sequence Synthetic oligonucleotide
actgtaggcaccatcaatc

SEQ ID NO: 2 - DNA, 17 bp, Artificial Sequence
  Description of Combined DNA/RNA Molecule Synthetic oligonucleotide
atcgtaggcaccugaua

SEQ ID NO: 3 - DNA, 36 bp, Artificial Sequence
  Description of Artificial Sequence Synthetic primer
gcctccctcgcgccatcagatcgtaggcacctgata

SEQ ID NO: 4 - DNA, 36 bp, Artificial Sequence
  Description of Artificial Sequence Synthetic primer
gccttgccagcccgctcagattgatggtgcctacag

SEQ ID NO: 5 - RNA, 25 nt, Artificial Sequence
  Description of Artificial Sequence Synthetic oligonucleotide
uacgcagaggccuaaguaaauaguc

SEQ ID NO: 6 - RNA, 26 nt, Artificial Sequence
  Description of Artificial Sequence Synthetic oligonucleotide
ccucugcguaggccauuuacuuuaag

SEQ ID NO: 7 - protein, 843 aa, Drosophila sp.
MADDQGRGRRRPLNEDDSSTSRGSGDGPRVKVFRGSSSGDPRADPRIEASRERRALEEAPRREGGPTERKPWGDQYDYLNTRPAELVSKKGTDGVPVMLQTNFFRLKTKPEWRIVHYHVEFEPSIENPRVRMGVLSNHANLLGSGYLFDGLQLFTTRKFEQEITVLSGKSKLDIEYKISIKFVGFISCAEPRFLQVLNLILRRSMKGLNLELVGRNLFDPRAKIEIREFKMELWPGYETSIRQHEKDILLGTEITHKVMRTETIYDIMRRCSHNPARHQDEVEVNVLDLIVLTDYNNRTYRINDVDFGQTPKSTFSCKGRDISFVEYYLTKYNIRIRDHNQPLLISKNRDKALKTNASELVVLIPELCRVTGLNAEMRSNFQLMRAMSSYTRMNPKQETDRLRAFNHRLQNTPESVKVLRDWNMELDKNVTEVQGRIIGQQNIVFHNGKVPAGENADWQRHFRDQRMLTTPSDGLDRWAVIAPQRNSHELRTLLDSLYRAASGMGLRIRSPQEFIIYDDRTGTYVRAMDDCVRSDPKLILCLVPNDNAERYSSIKKRGYVDRAVPTQVVTLKTTKNRSLMSIATKIAIQLNCKLGYTPWMIELPLSGLMTIGFDIAKSTRDRKRAYGALIASMDLQQNSTYFSTVTECSAFDVLANTLWPMIAKALRQYQHEHRKLPSRIVFYRDGVSSGSLKQLFEFEVKDIIEKLKTEYARVQLSPPQLAYIVVTRSMNTRFFLNGQNPPPGTIVDDVITLPERYDFYLVSQQVRQGTVSPTSYNVLYSSMGLSPEKMQKLTYKMCHLYYNWSGTTRVPAVCQYAKKLATLVGTNLHSIPQNALEKKFYYL

SEQ ID NO: 8 - protein, 866 aa, Drosophila sp.
MNLPPNPVIARGRGRGRKPNNVEANRGFAPSLGQKSDPSHSEGNQASGGNGGGGDAQVGPSIEKSSLSAVQMHKSEGDPRGSVRGRRLITDLVYSRPPGMTSKKGVVGTHITVQANYFKVLKRPNWTIYQYRVDFTPDVEATRLRRSFLYEHKGILGGYIFDGTNMFCINQFKAVQDSPYVLELVTKSRAGENIEIKIKAVGSVQSTDAEQFQVLNLILRRAMEGLDLKLVSRYYYDPQAKINLENFRMQLWPGYQTSIRQHENDILLCSEICHKVMRTETLYNILSDAIRDSDDYQSTFKRAVMGMVILTDYNNKTYRIDDVDFQSTPLCKFKTNDGEISYVDYYKKRYNIIIRDLKQPLVMSRPTDKNIRGGNDQAIMIIPELARATGMTDAMRADFRTLRAMSEHTRLNPDRRIERLRMFNKRLKSCKQSVETLKSWNIELDSALVEIPARVLPPEKILFGNQKIFVCDARADWTNEFRTCSMFKNVHINRWYVITPSRNLRETQEFVQMCIRTASSMKMNICNPIYEEIPDDRNGTYSQAIDNAAANDPQIVMVVMRSPNEEKYSCIKKRTCVDRPVPSQVVTLKVIAPRQQKPTGLMSIATKVVIQMNAKLMGAPWQVVIPLHGLMTVGFDVCHSPKNKNKSYGAFVATMDQKESFRYFSTVNEHIKGQELSEQMSVNMACALRSYQEQHRSLPERILFFRDGVGDGQLYQVVNSEVNTLKDRLDEIYKSAGKQEGCRMTFIIVSKRINSRYFTGHRNPVPGTVVDDVITLPERYDFFLVSQAVRIGTVSPTSYNVISDNMGLNADKLQMLSYKMTHMYYNYSGTIRVPAVCHYAHKLAFLVAESINRAPSAGLQNQLYFL

SEQ ID NO: 9 - protein, 867 aa, Drosophila sp.
MSGRGNLLSLFNKNAGNMGKSISSKDHEIDSGLDFNNSESSGERLLSSHNIETDLITTLQHVNISVGRGRARLIDTLKTDDHTSNQFITSESKENITKKTKGPESEAIASENGLFFPDLIYGSKGSSVNIYCNYLKLTTDESKGVFNYEVRFFPPIDSVHLRIKYLNDHKDKLGGTKTFDGNTLYLPILLPNKMTVFISKAEDVELQIRILYKKKEEMRNCTQLYNILFDRVMKVLNYVKFDRKQFDPSRPKIIPLAKLEVWPGYVTAVDEYKGGLMLCCDVSHRILCQKTVLEMLVDLYQQNVEHYQESARKMLVGNIVLTRYNNRTYKINDICFDQNPTCQFEIKTGCTSYVEYYKQYHNINIKDVNQPLIYSIKKSRGIPAERENLQFCLIPELCYLTGLRDEVRSDNKLMREIATFTRVSPNQRQMALNKFYENVSNTPAAQEILNSWGLSLTNNSNKISGRQMDIEQIYFSKISVSAGRSAEFSKHAVTNEMLKVVHLSKWIIIHLRNYRQAATSLLDNMKQACESLGMNISNPTMISLDHDRIDAYIQALRRNITMNTQMVVCICHNRRDDRYAAIKKICCSEIPIPSQVINAKTLQNDLKIRSVVQKIVLQMNCKLGGSLWTVKIPFKNVMICGIDSYHDPSNRGNSVAAFVASINSSYSQWYSKAVVQTKREEIVNGLSASFEIALKMYRKRNGKLPTNIIIYRDGIGDGQLYTCLNYEIPQFEMVCGNRIKISYIVVQKRINTRIFSGSGIHLENPLPGTVVDQHITKSNMYDFFLVSQLVRQGTVTPTHYVVLRDDCNYGPDIIQKLSYKLCFLYYNWAGTVRIPACCMYAHKLAYLIGQSIQRDVAEALSEKLFYL